SEQUENCE LISTING (5 sequences)

SEQ ID NO: 1 - DNA, 19 bp, synthetic construct
  misc_feature 1..19: note = Synthetic Sequence
  source 1..19: mol_type = other DNA
ttactaagcacaacactct

SEQ ID NO: 2 - DNA, 19 bp, synthetic construct
  misc_feature 1..19: note = Synthetic Sequence
  source 1..19: mol_type = other DNA
tacacattactttgaatcc

SEQ ID NO: 3 - DNA, 19 bp, synthetic construct
  misc_feature 1..19: note = Synthetic Sequence
  source 1..19: mol_type = other DNA
aactgagaaactaatgcct

SEQ ID NO: 4 - DNA, 19 bp, synthetic construct
  misc_feature 1..19: note = Synthetic Sequence
  source 1..19: mol_type = other DNA
ttcacagctacatcaacct

SEQ ID NO: 5 - DNA, 19 bp, synthetic construct
  misc_feature 1..19: note = Synthetic Sequence
  source 1..19: mol_type = other DNA
tttcttgcaaatgcattcg

(19) United States
(12) Patent Application Publication
(10) Pub. No.: US 2024/0400572 A1
(43) Pub. Date: Dec. 5, 2024

(54) COMPOUNDS AND METHODS FOR TREATING CANCERS THAT ARE MGMT DEFICIENT REGARDLESS OF MMR STATUS

(71) Applicant: Yale University, New Haven, CT (US)

(72) Inventors: Seth Herzon, New Haven, CT (US); Ranjit Bindra, New Haven, CT (US); Kingson Lin, New Haven, CT (US); Kyle Tarantino, New Haven, CT (US)

(21) Appl. No.: 18/694,284

(22) PCT Filed: Sep. 22, 2022
(86) PCT No.: PCT/US2022/076865

§ 371 Date: Mar. 21, 2024

Related U.S. Application Data
(60) Provisional application No. 63/247,645, filed on Sep. 23, 2021.
(60) Provisional application No. 63/290,630, filed on Dec. 16, 2021.
(60) Provisional application No. 63/332,945, filed on Apr. 20, 2022.

(51) Int. Cl. C07D 487/04; A61K 31/4188; A61K 31/427; A61K 31/444; A61K 31/496; A61P 35/00

(52) U.S. Cl. CPC C07D 487/04; A61K 31/4188; A61K 31/427; A61K 31/444; A61K 31/496; A61P 35/00

(57) ABSTRACT

Disclosed are compounds and methods of treating, ameliorating, and/or preventing cancers, including cancers that are MGMT deficient regardless of their MMR status, and particularly compounds and methods of treating, ameliorating, and/or preventing cancers that are both MGMT and MMR deficient or that are MGMT deficient and resistant to treatment with temozolomide.

### CROSS-REFERENCE TO RELATED APPLICATIONS

This application claims priority to U.S. Provisional Patent Application No. 63/247,645, filed Sep. 23, 2021, U.S. Provisional Patent Application No. 63/290,630, filed Dec. 16, 2021, and U.S. Provisional Patent Application No. 63/332,945, filed Apr. 20, 2022, the contents of each of which are hereby incorporated by reference herein in their entireties.

### STATEMENT REGARDING FEDERALLY SPONSORED RESEARCH OR DEVELOPMENT

This invention was made with government support under GM007205, CA254158, CA215453, and GM131913 awarded by National Institutes of Health. The government has certain rights in the invention.

### INCORPORATION-BY-REFERENCE OF MATERIAL SUBMITTED AS A TEXT FILE

This invention contains one or more sequences in a computer readable format in an accompanying text file titled “047162-7332WO1_ST26.xml,” which was created Sep. 18, 2022 and is 8 KB in size, the contents of which are incorporated herein by reference in their entirety.

### BACKGROUND

The present disclosure relates to compounds and methods for treating cancers that are MGMT deficient and particularly those that are also MMR deficient and/or resistant to temozolomide treatment.

The following discussion is provided merely to aid the reader in understanding the disclosure and is not admitted to describe or constitute prior art thereto.

Genetic instability is a hallmark of cancer and typically arises from mutations in key DNA damage repair and/or reversal proteins (collectively referred to herein as the DNA damage response, (DDR). Intrinsic DDR defects can be exploited with DDR inhibitors via the concept of synthetic lethality, defined as a loss of viability resulting from the disruption of two genes or pathways, which, if disrupted individually, are non-lethal. Notable examples of synthetic lethal interactions between DDR inhibitors and key tumor-associated DDR defects include: (1) homologous recombination (HR)-defective tumors and inhibitors of poly(ADP)-ribose polymerase (PARP) and polymerase theta (Pol θ); (2) ataxia-telangiectasia mutated (ATM)-mutant tumors and ataxia telangiectasia and Rad3-related (ATR) inhibitors; and (3) mismatch repair (MMR)-deficient tumors and Werner syndrome helicase (WRN) inhibitors. In each of these examples, selective tumor cell killing via the DDR protein inhibitor relies on either the induction or persistence of DNA damage or aberrant DNA structures.

Approximately half of glioblastoma multiforme (GBM) and over two-thirds of grade II/III glioma tumors lack the DNA repair protein O⁶-methylguanine methyl transferase (MGMT) via promoter hypermethylation. GBM is the most common and devastating form of brain cancer, with a five year survival rate of ˜5%.

Monofunctional alkylators, such as temozolomide (TMZ), act by alkylating the O⁶ guanine in a cell's DNA, thus preventing effective replication and killing the cell. Patients with MGMT-deficient tumors (referred to hereafter as MGMT− tumors) are treated with temozolomide (TMZ, 1a), a prodrug that converts under physiological conditions to the potent methylating agent methyl diazonium (1c), via the intermediacy of 3-methyl-(triazen-1-yl)-imidazole-4-carboxamide (MTIC, 1b) (FIG. Z1B). N7-Methylguanosine and N3-methyladenosine are the major sites of methylation (70% and 9% respectively) but are readily resolved by the base excision repair (BER) pathway. MGMT− tumors respond initially to the DNA methylation agent temozolomide, but frequently acquire resistance via loss of the mismatch repair (MMR) pathway.

Such alkylators are typically effective only in cells which have below normal expression of the DNA repair protein MGMT (O⁶-methylguanine-DNA-methyltransferase). These cells are termed “MGMT deficient”. In cells which express normal levels of MGMT, the enzyme can reverse the alkylation and restore the affected DNA to its pre-alkylation status. Since expression of MGMT is frequently lost in tumorigenesis, monofunctional alkylators can differentially kill cancer cells that lack this repair protein while MGMT proficient non-cancerous cells can survive. This principle is known clinically as therapeutic index.

In addition to mutations in MGMT, cancers also often develop mutations in mismatch repair (MMR) genes, either as a consequence of treatment or during normal tumorigenesis. Many cancers treated with TMZ develop mutations in MMR genes and become MMR deficient. It is now well-established (15) that acquired clinical resistance to TMZ (1a) by MMR mutations abrogates its toxicity, leading to recurrent GBM and death in nearly all patients. TMZ (1a) is also frequently utilized as adjuvant therapy for grade III and high-risk grade II gliomas; however, it remains non-curative, with recurrences typically occurring over 2-10 years. In approximately 50-80% of patients, recurrences coincide with transformation to higher grade tumors resistant to TMZ (1a) and harboring a distinct hypermutation signature secondary to MMR deficiency, resulting in reduced survival (16, 17). Because these cancers having MMR mutations are resistant to monofunctional alkylators such as TMZ, such alkylators do not provide an effective therapeutic regimen for treating such cancers.

Although temozolomide was first introduced over twenty years ago, it remains first line therapy for treatment of glioblastoma despite the fact that it is ineffective in cancers that have MMR mutations, regardless of MGMT status. Temozolomide is the subject of Lund et al. U.S. Pat. No. 5,266,291. Many derivatives of TMZ have been described in the literature, including in U.S. Pat. Nos. 6,251,886; 6,987,108; 8,450,479; and 9,024,018; patent publications US 2021/0002286; GB 2,125,402; and WO 2009/077741.

Despite over twenty years of seeking improvement on temozolomide, no improved molecule has emerged. Compounds that have better activity against cancer than temozolomide would be of great medical value, as would a therapy that treats cancers that are MGMT deficient and either have an MMR mutation causing TMZ resistance or are MMR deficient.

Thus, there is a need in the art for agents that can overcome this resistance mechanism by inducing MMR-independent cell kill selectively in MGMT-silenced tumors and methods of use thereof. The present disclosure addresses this need.

### BRIEF SUMMARY OF THE DISCLOSURE

Non-limiting aspects of this disclosure provide compounds, pharmaceutical compositions, and methods for treating cancer, such as a gliobastoma.

In one non-limiting aspect, provided herein is a compound of formula (I) or a salt thereof:

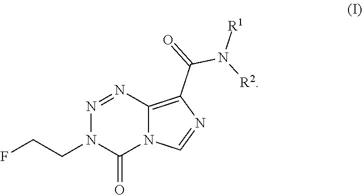

In certain embodiments, R¹ and R² are each independently selected from H and lower alkyl. In certain embodiments, R¹ and R² combine to form —(CH₂)ₙ—. In certain embodiments, n is 2, 3, 4, or 5. In certain embodiments, R¹ and R² are not simultaneously H. Additional description of exemplary specific compounds is provided herein. The compounds may be part of a pharmaceutical composition. The compounds may be used in methods of treatment described herein.

Exemplary benefits of multiple compounds described herein are illustrated in the working examples. For instance, compound I-1 was found to achieve a much larger concentration in brain tissue upon oral administration to mice than compound KL50. Additionally, compound I-1 was found to result in a longer duration of survival in mice bearing tumors formed from LN-229 human brain gliobastoma cells, compared to such mice treated with KL50 or TMZ.

In another non-limiting aspect, provided herein is a method of treating, preventing, and/or ameliorating cancer in a subject in need thereof. In certain embodiments, the method comprises administering to the subject a therapeutically effective amount of a compound described herein, such as a compound of formula (I).

In another non-limiting aspect, provided herein is a method of treating, preventing, and/or ameliorating cancer in a subject in need thereof. In certain embodiments, the method comprises administering to the subject a therapeutically effective amount of a compound described herein (e.g., a compound of formula (I)), wherein the cancer is MGMT deficient and either MMR deficient or refractory to treatment with temozolomide.

In another non-limiting aspect, provided herein is a method of treating, preventing, and/or ameliorating cancer in a subject in need thereof, where the cancer is characterized by a cancer cell having altered MGMT activity. The method comprises administering to the subject an agent that induces DNA lesions in the cell that lead to irreparable DNA damage to selectively treat the cancer. In certain embodiments, the agent does not affect MGMT proficient tissue. In certain embodiments, the agent activity is independent of MMR protein expression and/or functional activity of the MMR pathway.

In certain embodiments, “altered MGMT activity” as used herein refers to downregulation of MGMT activity. In certain embodiments, “altered MGMT activity” as used herein refers to upregulation of MGMT activity. In certain embodiments, “altered MGMT activity” as used herein refers to upregulation or downregulation of MGMT activity. In certain embodiments, the downregulation or upregulation of MGMT activity is relative to MGMT activity in a healthy (non-cancerous) cell of the subject or patient.

In certain embodiments, the cancer is selected from the group consisting of a glioma, colorectal cancer, non-small cell lung cancer, small cell lung cancer, sarcoma, pancreatic cancer, neuroendocrine tumor, esophageal cancer, lymphoma, head and neck cancer, breast cancer, bladder cancer, and leukemia. In certain embodiments, the cancer is selected from the group consisting of an anaplastic astrocytoma, anaplastic oligodendroglioma, anaplastic ependymoma, medulloblastoma, and glioblastoma.

In certain embodiments, the agent is an imidazotetrazine-based compound or a triazine-based compound. In certain embodiments, the DNA lesion is a DNA double-strand break, a single-strand break, a stalled replication fork, a bulky adduct, or a lesion that further chemically reacts to form irreparable DNA damage. In certain embodiments, the irreparable DNA damage can be unrepaired lesions such as DNA inter- or intra-strand crosslinks.

In an aspect, provided herein is a method of treating, preventing, and/or ameliorating cancer in a subject in need thereof, wherein the cancer is charactered by a cancer cell having altered MGMT expression, wherein the method comprises administering to the subject an agent that induces DNA lesions in the cell that lead to irreparable DNA damage to selectively treat the cancer. In certain embodiments, the agent does not affect MGMT proficient tissue. In certain embodiments, the cancer is selected from the group consisting of a glioma, colorectal cancer, non-small cell lung cancer, small cell lung cancer, sarcoma, pancreatic cancer, neuroendocrine tumor, esophageal cancer, lymphoma, head & neck cancer, breast cancer, bladder cancer, and leukemia. In certain embodiments, the cancer is selected from the group consisting of an anaplastic astrocytoma, anaplastic oligodendroglioma, anaplastic ependymoma, medulloblastoma, and glioblastoma. In certain embodiments, the agent is an imidazotetrazine-based compound or a triazine-based compound. In certain embodiments, the DNA lesion is a DNA double-strand break, a single-strand break, a stalled replication fork, a bulky adduct, or a lesion that further chemically reacts to form irreparable DNA damage. In certain embodiments, the irreparable DNA damage can be unrepaired lesions such as DNA inter- or intra-strand crosslinks.

In certain embodiments, “altered MGMT expression” as used herein refers to downregulation of MGMT expression. In certain embodiments, “altered MGMT expression” as used herein refers to upregulation of MGMT expression. In certain embodiments, “altered MGMT expression” as used herein refers to upregulation or downregulation of MGMT expression. In certain embodiments, the downregulation or upregulation of MGMT expression is relative to MGMT expression in a healthy (non-cancerous) cell of the subject or patient.

In certain embodiments, the subject is resistant to treatment with an antineoplastic agent. In certain embodiments, the antineoplastic agent is selected from temozolomide, procarbazine, altretamine, dacarbazine, mitozolomide, cisplatin, carboplatin, dicycloplatin, eptaplatin, lobaplatin, oxaliplatin, miriplatin, nedaplatin, triplatin tetranitrate, phenanthriplatin, Picoplatin, satraplatin, and lomustine.

In certain embodiments, the agent that induces DNA lesions in the cell is a compound of formula II, Ia, Ib, or Ic.

In another aspect, provided herein is a method of treating, preventing, and/or ameliorating cancer in a patient, the cancer being MGMT deficient, the method comprising administering to said patient a therapeutically-effective dose of a compound of formula (II):

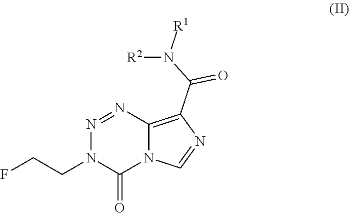

or a pharmaceutically acceptable salt thereof. In certain embodiments, R¹ is individually selected from H and lower alkyl. In certain embodiments, R² is individually selected from H, lower alkyl, trifluoroethyl,

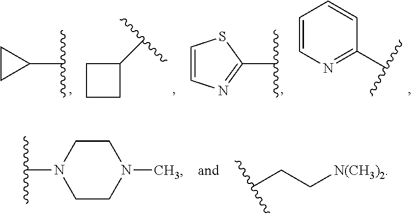

In certain embodiments, R¹ is H when R² is other than H or lower alkyl. In certain embodiments, R¹ and R² are not both H. In certain embodiments, R¹ and R² may combine to form —(CH₂)ₙ—. In certain embodiments, n is 3, 4, or 5. In certain embodiments, R¹ and R² may combine to form —(CH₂)₂—N(CH₃)—(CH₂)₂—. The disclosed compounds are useful to treat cancers that are MGMT deficient regardless of MMR status.

In certain embodiments, the cancer is also either MMR deficient or refractory (unresponsive) to treatment by temozolomide.

As described in more detail elsewhere herein, the compounds of formula (II) have a high therapeutic index for MGMT deficient cell lines that are either MMR⁺ or MMR⁻, but particularly those that are MMR⁻. TMZ, on the contrary, has a poor therapeutic index for cells that are MMR⁻, regardless of their MGMT status. Since temozolomide is sensitive to minor changes in MMR proteins, the compounds of formula (II) are useful to treat cancer that is MGMT deficient and does not respond to treatment by temozolomide.

The disclosure also provides certain novel compounds, such as represented by formula (II). These compounds are more effective anti-cancer compounds than temozolomide against MGMT deficient cancers regardless of MMR status, as well as being effective against cancers that are both MGMT and MMR deficient and cancers that are MGMT deficient and refractory to treatment by temozolomide.

In another aspect, provided herein is a compound of formula (II)

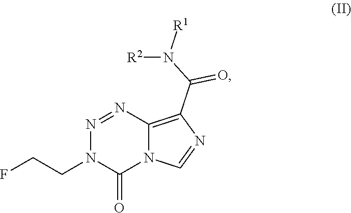

or a pharmaceutically acceptable salt thereof. In certain embodiments, R¹ is individually selected from H and lower alkyl. In certain embodiments, R² is individually selected from H, lower alkyl, trifluoroethyl,

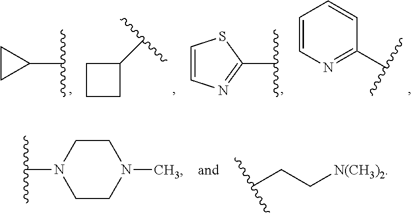

In certain embodiments, R¹ is H when R² is other than H or lower alkyl. In certain embodiments, R¹ and R² are not both H. In certain embodiments, R¹ and R² may combine to form —(CH₂)ₙ—. In certain embodiments, n is 3, 4, or 5. In certain embodiments, R¹ and R² may combine to form —(CH₂)₂—N(CH₃)—(CH₂)₂—.

### BRIEF DESCRIPTION OF THE DRAWINGS

FIG. 1 shows RNA-sequencing data identifying cancers that have significant sub-populations displaying reduced MGMT expression, where notable cancers are: bladder urothelial carcinoma, breast invasive cancer, colon adenocarcinoma, head and neck tumor, lung adenocarcinoma, lung squamous cell carcinoma, rectum adenocarcinoma, glioblastoma multiforme, brain lower grade glioma, and acute myeloid leukemia. Each dot represents an individual patient sample.

FIG. 2 shows a general synthetic route to derivatives of TMZ.

FIG. 3 shows the structures of certain TMZ derivatives prepared.

FIG. 4 is a graph showing mean plasma concentration-time profiles and brain concentration-time profiles of KL-50 after single PO dose at 20 mg/kg in male C57BL/6 mice (N=3/timepoint), as further described in Example 20.

FIG. 5 is a graph showing mean plasma concentration-time profiles and brain concentration-time profiles of compound I-1 after single PO dose at 20 mg/kg in male C57BL/6 mice (N=3/timepoint), as further described in Example 20.

FIG. 6 is a graph showing the results of bioluminescence imaging for each group of mice according to study compound or vehicle, as further described in Example 21.

FIG. 7 is graph showing survival endpoint data for each group of mice according to study compound or vehicle, as further described in Example 21.

FIG. 8 shows IC₅₀ test result values and the resulting therapeutic indices of TMZ and multiple compounds from FIG. 3 in the short-term cell viability assay.

FIG. 9 shows clonogenic survival assays testing the activity of TMZ and derivative 10f (KL-50). Arrow indicators highlight TMZ resistance of MGMT−/MMR− cells, compared to the efficacy of KL50.

FIG. 10 shows dose related short-term cell survival results for KL50 and N-methyl KL50.

FIG. 11 shows in vivo xenograft test results for KL50 and TMZ.

FIG. 12 depicts graphs of results showing that KL-50 overcomes MMR mediated resistance in colorectal cell lines.

FIG. 13 depicts graphs of results showing that KL-50 is pan-MMR independent.

FIG. 14 depicts graphs of results showing that KL-50 is pan-MMR independent.

FIG. 15 depicts graphs of results showing that KL-50 is a MGMT dependent alkylator that overcomes MMR mediated resistance.

FIG. 16 depicts graphs of results showing performance of KL-50 and TMZ in patient derived glioblastoma multiforme cell lines.

FIG. 17 depicts graphs of results showing KL-50 to be efficacious and safe in vivo in flank models.

FIG. 18 depicts graphs of results showing that KL-50 potently suppresses the growth of tumors.

FIG. 19 depicts a graph and table of results from studies measuring the maximum tolerated dose of KL-50.

FIG. 20 depicts graphs of results from studies measuring CNS penetration of KL-50 and survival of tumor bearing mice that have been administered KL-50 or TMZ.

FIGS. Z1A-1ZF. Overview of mechanistic strategy and structures of agents employed in this study. (FIG. ZIA) Underlying mechanistic hypothesis. Systemic administration of a bifunctional agent is envisioned to form a primary lesion that is rapidly resolved by healthy (DDR+) but not DDR-deficient (DDR−) cells. The persistence of the primary lesion allows it to evolve slowly to a more toxic secondary lesion. (FIG. Z1B) TMZ (1a) is the front-line therapy for the treatment of MGMT− GBM. Under physiological conditions, TMZ (1a) converts to MTIC (1b) which decomposes to methyl diazonium (1c). (FIG. Z1C) O⁶-Guanine is the most clinically-significant site of methylation by methyl diazonium (1c). O⁶MeG (3) is rapidly reverted to dG (2) by MGMT (the second-order rate constant for demethylation of calf thymus DNA by MGMT is 1×10⁹ M⁻¹·min⁻¹), but persists in the genome of MGMT-cells, ultimately leading to MMR-dependent cytotoxicity. (FIG. Z1D) We envisioned that we could utilize the imidazotetrazine KL-50 (4a) as a source of 2-fluoroethyl diazonium ion (4c). (FIG. Z1E) Fluoroethylation at O⁶-guanosine would form O⁶FEtG (5), which is known to slowly rearrange (t_{1/2}˜18.5 h at 37° C.) via intermediate 6 to form the dG-dC ICL 8. Based on the broad substrate scope of MGMT, we anticipated that O⁶FEtG (5) would be readily reversed in MGMT+ cells, thereby preventing ICL formation in healthy tissue. Realization of this goal would provide the first MMR-independent agent active specifically in MGMT-glioma. (FIG. Z1F) Structures of the triazenes 9-13, mitozolomide 12a, and lomustine (CCNU, 14).

FIGS. Z2A-Z2H. KL-50 (4a) displays novel MGMT-dependent, MMR-independent cytotoxicity in multiple isogenic cell models. (FIG. Z2A) IC₅₀ values derived from short-term viability assays in LN229 MGMT+/−, MMR+/− cells treated with TMZ (1a) derivatives. ^{a}MGMT TI (therapeutic index)=IC₅₀ (MGMT+/MMR+) divided by IC₅₀ (MGMT−/MMR+). ^{b}MMR RI (resistance index)=IC₅₀ (MGMT−/MMR−) divided by IC₅₀ (MGMT−/MMR+). (FIG. Z2B) Short-term viability assay curves for TMZ (1a), CCNU (14), KL-85 (4b), and KL-50 (4a) in LN229 MGMT+/−, MMR+/− cells. (FIG. Z2C) Clonogenic survival curves for TMZ (1a) in LN229 MGMT+/−, MMR+/− cells, with representative images of wells containing 1000 plated cells treated with 30 μM TMZ (1a). (FIG. Z2D) Clonogenic survival curves for KL-50 (4a) in LN229 MGMT+/−, MMR+/− cells, with representative images of wells containing 1000 plated cells treated with 30 μM KL-50 (4a). (FIG. Z2E) Short-term viability assay curves for TMZ (1a) in DLD1 MSH6-deficient cells pre-treated with 0.01% DMSO control (CTR) or 10 μM O⁶BG (+O⁶BG) for 1 h prior to TMZ (1a) addition to deplete MGMT. (FIG. Z2F) Short-term viability assay curves for KL-50 (4a) in DLD1 MSH6-deficient cells pre-treated with 0.01% DMSO control (CTR) or 10 μM O⁶BG (+O⁶BG) for 1 h prior to KL-50 (4a) addition. (FIG. Z2G) Short-term viability assay curves for TMZ (1a) in HCT116 MLH1−/− cells or HCT116 cells complemented with chromosome 3 carrying wildtype MLH1 (+Chr3) pre-treated with 0.01% DMSO control or 10 μM O⁶BG (+O⁶BG) for 1 h prior to TMZ (1a) addition. (FIG. Z2H) Short-term viability assay curves for KL-50 (4a) in HCT116 MLH1−/− cells or HCT116 cells complemented with chromosome 3 carrying wildtype MLH1 (+Chr3) pre-treated with 0.01% DMSO control or 10 μM O⁶BG (+O⁶BG) for 1 h prior to KL-50 (4a) addition. For (FIG. Z2B), (FIG. Z2C), (FIG. Z2D), (FIG. Z2E), (FIG. Z2F), (FIG. Z2G), and (FIG. Z2H), points, mean; error bars, SD; n≥3 technical replicates.

FIGS. Z3A-Z3F. Unrepaired primary KL-50 (4a) lesions convert to DNA ICLs in the absence of MGMT. (FIG. Z3A) Scatter dot plots of the % DNA in tail upon single cell alkaline gel electrophoresis performed on LN229 MGMT−/MMR+ and MGMT−/MMR− cells treated with 0.2% DMSO control, 200 μM TMZ (1a), 200 μM KL-50 (4a), or 0.1 μM MMC (MMC*) for 24 h or with 50 μM MMC (MMC**) for 2 h. After cell lysis, comet slides were irradiated with 0 or 10 Gy prior to alkaline electrophoresis. Lines, median; error bars, 95% CI; n≥160. (FIG. Z3B) Representative comet images from (FIG. Z3A). (FIG. Z3C) Scatter dot plots of the % DNA in tail upon single cell alkaline gel electrophoresis performed on LN229 MGMT−/MMR− cells treated with 0.2% DMSO control, 200 μM MTZ (12a), 200 μM TMZ (1a), or 200 μM KL-50 (4a) for 2, 8, or 24 h. After cell lysis, comet slides were irradiated with 10 Gy prior to alkaline electrophoresis. Lines, median; error bars, 95% CI; n≥230. Data from samples treated with 0 Gy are shown in FIGS. ZS4C and ZS4D. (FIG. Z3D) Representative comet images from (FIG. Z3C). (FIG. Z3E) Denaturing gel electrophoresis of genomic DNA isolated from LN229 MGMT−/MMR+ cells treated with 0.2% DMSO control, 200 μM KL-50 (4a), 200 μM TMZ (1a), 200 μM KL-85 (4b), or 200 M MTIC (1b) for 24 h or with 50 μM MMC or 200 μM CCNU (14) for 2 h. (FIG. Z3F) Denaturing gel electrophoresis of linearized 100 ng pUC19 plasmid DNA treated in vitro with 100 μM Cisplatin (36 hours), 100 μM MMS (36 hours), 100 μM of KL-50 (4a) or 12b for 6-36 hours. For (FIG. Z3E) and (FIG. Z3F), bands representing crosslinked DNA are indicated by arrows.

FIGS. Z4A-Z4I. KL-50 (4a) activates DNA damage response pathways and cycle arrest in MGMT− cells, independent of MMR, and induces sensitivity in cells deficient in ICL or HR repair. (FIGS. Z4A, Z4B, and Z4C) Phospho-SER139-H2AX (γH2AX) (A), 53BP1 (FIG. Z4B), and phospo-SER33-RPA2 (pRPA) (FIG. Z4C) foci formation quantified by % cells with 10 foci in LN229 MGMT+/−, MMR+/− cells treated with 0.1% DMSO control, 20 μM KL-50 (4a), or 20 μM TMZ (1a) for 48 h. Columns, mean; error bars, SD; n≥5 technical replicates. Additional time course data is presented in FIG. ZS6B to D. (FIG. Z4D) Representative foci images of data in (FIG. Z4A) to (FIG. Z4C). (FIG. Z4E) Percentage of cells in G1, S, and G2 cell cycle phases after treatment as in (A) to (C), measured using integrated nuclear (Hoechst) staining intensity. Columns, mean; error bars, SD; n=3 independent analyses. Additional time course data, cell cycle controls, and representative histograms are presented in FIG. ZS7. (FIG. Z4F) Change in percent cells with 1 micronuclei from baseline (DMSO control) after treatment as in (FIG. Z4A) to (FIG. Z4C). Columns, mean; error bars, SD; n 15 technical replicates; **** p<0.0001; ns, not significant. Additional validation is presented in FIG. ZS9, A and B. (FIG. Z4G) Short-term viability assay curves for KL-50 (4a) in PD20 cells, deficient in FANCD2 (FANCD2−/−) or complemented with FANCD2 (+FANCD2). (FIG. Z4H) Short-term viability assay curves for KL-50 (4a) in PEO4 (BRCA2+) and PEO1 (BRCA2−/−) cells pre-treated with 0.01% DMSO control or 10 μM O⁶BG (+O⁶BG) for 1 h prior to KL-50 (4a) addition. (FIG. Z4I) Short-term viability assay curves for KL-50 (4a) in DLD1 BRCA2+/− and BRCA2−/− cells pre-treated with 0.01% DMSO control or 10 μM O⁶BG (+O⁶BG) for 1 h prior to KL-50 (4a) addition. For (FIG. Z4G), (FIG. Z4H), and (FIG. Z4I), points, mean; error bars, SD; n=3 technical replicates.

FIGS. Z5A-Z5F. KL-50 (4a) is safe and efficacious on both MGMT−/MMR+ and MGMT−/MMR− flank tumors over a wide range of treatment regimens and conditions. (FIG. Z5A) Xenograft LN229 MGMT−/MMR+ flank tumors treated with 3 weekly cycles of P.O. administration of 10% cyclodextrin control (n=7), TMZ (1a) (n=7, 5 mg/kg) or KL-50 (4a) (n=6, 5 mg/kg) on Monday, Wednesday, and Friday (individual spider plots in FIG. ZS10A). (FIG. Z5B) Xenograft LN229 MGMT−/MMR− flank tumors treated with 3 weekly cycles of P.O. administration of 10% cyclodextrin control (n=6), TMZ (1a) (n=5, 5 mg/kg) or KL-50 (4a) (n=5, 5 mg/kg) on Monday, Wednesday, and Friday (individual spider plots in FIG. ZS10B). (FIG. Z5C) Mean body weight of mice during LN229 flank tumor experiments. (FIG. Z5D) Kaplan-Meier analysis of LN229 MGMT−/MMR− xenograft flank tumor-bearing mice to determine survival rate based on death, removal from study if mouse body weight loss exceeded 20% of initial body weight, or if tumor volume exceeded 2000 mm³. Both control and TMZ (1a) treated groups have a median OS of 10 weeks and KL-50 (4a) treated mice have median OS of greater than 15 weeks. (FIG. Z5E) Xenograft LN229 MGMT−/MMR+ and LN229 MGMT−/MMR− flank tumors treated with PO administration of 10% cyclodextrin control (n=7), KL-50 (4a) (n=6, 3 cycles of 15 mg/kg on Monday, Wednesday, Friday), KL-50 (4a) (n=6, 1 cycle of 25 mg/kg Monday through Friday), or intraperitoneal (I.P.) administration of KL-50 (4a) (n=7, 3 cycles of 5 mg/kg on Monday, Wednesday, Friday) revealed equal efficacy with no observable increases in toxicity as measured by mice systemic weights (individual spider plots in FIG. ZS10C and FIG. ZS10D). (FIG. Z5F) Xenograft LN229 MGMT−/MMR+ and LN229 MGMT−/MSH6− flank tumors with a larger average starting tumor size of ˜400 mm³ and ˜350 mm³ respectively, treated with 3 weekly cycles of P.O administration of 10% cyclodextrin (n=4) or KL-50 (4a) (n=4, 3 cycles of 25 mg/kg on Monday, Wednesday, and Friday). The study period was limited by control groups which had to be euthanized for exceeding the ethical maximum allowed tumor size, thus ending the study. In all panels, points, mean; error bars, SEM; *, P<0.05; **, P<0.01; *** P<0.001; ****, P<0.0001; ns, not significant.

FIGS. Z6A-Z6C. KL-50 (4a) is efficacious in an LN229 MGMT−/MMR− intracranial model and is well tolerated with limited myelosuppression at supratherapeutic doses. (FIG. Z6A) Mean tumor size as measured by bioluminescent imaging (BLI) as relative light units (RLU; photons/sec) with SEM of xenograft LN229 MGMT−/MMR− intracranial tumors treated with 3 weekly cycles of P.O administration with 10% cyclodextrin control (n=10), TMZ (1a) (n=11, 25 mg/kg) or KL-50 (4a) (n=11, 25 mg/kg) on Monday, Wednesday, and Friday (individual spider plots in FIG. ZS10E). (FIG. Z6B) Mean body change with SEM of mice during maximum tolerated dose experiment in non-tumor bearing mice. (FIG. Z6C) Complete blood counts for mice pre-treatment and 7 days post-treatment with escalations of single dose KL-50 (4a) delivered PO. WBC lower limit of normal (LLN): 2.2 K/L; Neutrophils LLN: 0.42 K/L; Lymphocyte LLN: 1.7 K/L; RBC LLN: 3.47 M/L; Platelet LLN: 155 K/L. *, P<0.05; ****, P<0.0001.

FIGS. ZS1A-ZS1C. Literature precedent for the hydrolysis of various 2-haloethylguanosine lesions. (FIG. ZS1A) Kinetics of the hydrolysis of 06-(2-fluoroethylguanosine) (S1) at pH 7.4 and 37° C. as reported by Tong et al. (18). (FIG. ZS1B) Kinetics of the hydrolysis of 06-(2-chloroethylguanosine) (S4) at pH 7.4 and 37° C. as reported by Parker et al. (39). (FIG. ZS1C) Failed hydrolysis ofN7-(2-fluoroethyl)guanosine (S5) with extensive incubation of [S5] at 37° C. in neutral aqueous solution.

FIGS. ZS2A-ZS2K. Additional analysis of TMZ (1a) derivatives in MGMT+/−, MMR+/− cell models. (FIG. ZS2A) Western blotting performed in LN229 MGMT−/MMR+ parental line, and cells complemented with wildtype MGMT (MGMT+/MMR+) and/or stable expression of MSH2 shRNA (MGMT+/MMR− and MGMT−/MMR−). MSH6 expression is reduced in these lines due to destabilization in the setting of loss of its heterodimeric partner MSH2. MLH1 expression is not affected by MSH2 knockdown. Vinculin serves as loading control. (FIG. ZS2B, FIG. ZS2C, FIG. ZS2D, FIG. ZS2E, FIG. ZS2F, and FIG. ZS2G) Short-term viability assay curves for compounds 9, 10, 11, 12b, 13, and 12a in LN229 MGMT+/−, MMR+/− cells. (FIG. ZS2H) Clonogenic survival curves for lomustine (14) in LN229 MGMT+/−, MMR+/− cells. (FIG. ZS2I) Western blotting in HCT116 and DLD1 cells. HCT116 MLH1−/− and +Chr3 lines demonstrate re-expression of MLH1 and similar levels of MGMT and other MMR proteins. DLD1 BRCA2+/− and BRCA2−/− cells have known loss of MSH6 but comparable levels of MGMT and other MMR protein expression. GAPDH serves as loading control. (FIG. ZS2J) Western blotting performed in HCT116 MLH1−/− and +Chr3 and DLD1 BRCA2+/− and BRCA2−/− cells after exposure to 0.01% DMSO or 10 μM O6BG for 24 h, demonstrating 06BG-induced MGMT depletion. Vinculin serves as loading control. (FIG. ZS2K) Short-term cell viability curves for KL-50 (4a) and TMZ (1a) in BJ fibroblast cells. For (FIG. ZS2B), (FIG. ZS2C), (FIG. ZS2D), (FIG. ZS2E), (FIG. ZS2F), (FIG. ZS2G), (FIG. ZS2H), and (FIG. ZS2K), points, mean; error bars, SD; n=3 technical replicates.

FIGS. ZS3A-ZS3J. KL-50 (4a) is effective in TMZ (1a)-resistant cells lacking other MMR proteins. (FIG. ZS3A) Western blotting performed in LN229 MGMT+/− cells with stable expression of shRNA targeting MSH6, MLH1, PMS2, or MSH3 to confirm depletion of the shRNA targets. In shMSH6 cells, there is reduced expression of MSH2, and in shMLH1 cells, there is loss of PMS2, due to destabilization in the setting of loss of their heterodimeric partners. GAPDH serves as loading control. (FIG. ZS3B) (Table S1.) IC50 values derived from short-term viability assays in LN229 MGMT+/− cells lines, +/−shRNA, treated with TMZ (1a) or KL-50 (4a). aMGMT TI (therapeutic index)=IC50 (MGMT+/MMR+) divided by IC50 (MGMT−/MMR+). bMMR RI (resistance index)=IC50 (MGMT−/MMR−) divided by IC50 (MGMT−/MMR+). (FIG. ZS3C) Short-term viability assay curves for TMZ (1a) in LN229 MGMT+/−, MMR+/shMSH6 cells. (FIG. ZS3D) Short-term viability assay curves for KL-50 (4a) in LN229 MGMT+/−, MMR+/shMSH6 cells. (FIG. ZS3E) Short-term viability assay curves for TMZ (1a) in LN229 MGMT+/−, MMR+/shMLH1 cells. (FIG. ZS3F) Short-term viability assay curves for KL-50 (4a) in LN229 MGMT+/−, MMR+/shMLH1 cells. (FIG. ZS3G) Short-term viability assay curves for TMZ (1a) in LN229 MGMT+/−, MMR+/shPMS2 cells. (FIG. ZS3H) Short-term viability assay curves for KL-50 (4a) in LN229 MGMT+/−, MMR+/shPMS2 cells. (FIG. ZS3I) Short-term viability assay curves for TMZ (1a) in LN229 MGMT+/−, MMR+/shMSH3 cells. (FIG. ZS3J) Short-term viability assay curves for KL-50 (4a) in LN229 MGMT+/−, MMR+/shMSH3 cells. For (FIG. ZS3C), (FIG. ZS3D), (FIG. ZS3E), (FIG. ZS3F), (FIG. ZS3G), (FIG. ZS3H), (FIG. ZS3I), and (FIG. ZS3J), points, mean; error bars, SD; n=3 technical replicates.

FIGS. ZS4A-ZS4D. Supplementary IR alkaline comet assay data. (FIG. ZS4A) Scatter dot plots of the % DNA in tail upon single cell alkaline gel electrophoresis performed on LN229 MGMT−/MMR+ cells treated with 0.1% DMSO control or 200 μM KL-85 (4b) for 24 h or with 50 μM MMC for 2 h. After cell lysis, comet slides were irradiated with 0 or 10 Gy prior to alkaline electrophoresis. Lines, median; error bars, 95% CI; n≥160. (FIG. ZS4B) Representative comet images from FIG. ZS4A. (FIG. ZS4C) Scatter dot plots of the % DNA in tail upon single cell alkaline gel electrophoresis performed on LN229 MGMT−/MMR-cells treated with 0.2% DMSO control, 200 μM MTZ (12a), 200 μM TMZ (1a), or 200 μM KL-50 (4a) for 2, 8, or 24 h. Corresponding samples treated with 10 Gy IR are shown in FIG. Z3C. Lines, median; error bars, 95% CI; n≥230. (FIG. ZS4D) Representative comet images from FIG. ZS4C.

FIGS. ZS5A-ZS5D. NER, BER, ROS, and altered DNA melting point do not play a major role in the mechanism of KL-50 (4a). (FIG. ZS5A) Short-term cell viability assays in both WT and XPA-deficient MEFs demonstrating the absence of additional sensitivity to KL-50 (4a) in NER compromised XPA deficient cells ±MGMT depletion with 06BG, in contrast to cisplatin as positive control. (FIG. ZS5B) EndoIV depurination assay utilizing supercoiled pUC19 plasmid DNA assessing both spontaneous and enzymatically catalyzed SSB formation resulting from depurination post-treatment, demonstrating comparable levels of depurination and SSB formation by KL-50 (4a) and TMZ (1a). (FIG. ZS5C) Short-term cell viability assays in LN229 MGMT+/−, MMR+/− isogenic lines pre-treated with increasing concentrations of the ROS scavenger NAC did not result in rescue of KL-50 (4a) toxicity. (FIG. ZS5D) Melting temperature experiments in linearized pUC19 plasmid DNA treated with 100 or 500 μM of MMS or KL-50 (4a) for 3 h resulted in comparable changes in measured DNA melting temperature. Columns, mean; error bars, SD; n=2 independent analyses. For (FIG. ZS5A) and (FIG. ZS5C), points, mean; error bars, SD; n=3 technical replicates.

FIGS. ZS6A-ZS6D. KL-50 (4a) induces activation of the ATR-CHK1 and ATM-CHK2 signaling axes and delayed DNA repair foci formation in MGMT-deficient cells, independent of MMR status. (FIG. ZS6A) Western blotting performed in LN229 MGMT+/−, MMR+/− cells following treatment with 20 μM KL-50 (4a) or TMZ (1a) for 24 or 48 h. Treatment with 1 μM doxorubicin for 24 h (Doxo) served as a positive control for p-CHK1 activation. (FIG. ZS6B and FIG. ZS6C) Phospho-SER139-H2AX (γH2AX), 53BP1, and phospho-SER33-RPA2 (pRPA) foci levels over time following treatment with KL-50 (4a; 20 μM) (B) or TMZ (1a; 20 μM) (C) for 0, 2, 8, 24, or 48 h in LN229 MGMT+/−, MMR+/− cells. Points, mean % cells with 10 foci; error bars, SD; n≥5 technical replicates. (FIG. ZS6D) Extended time course of γH2AX foci levels following treatment with KL-50 (4a; 20 μM) or TMZ (1a; 20 μM) for 0, 48, 72, or 96 h in LN229 MGMT+/−, MMR+/− cells. Points, % cells with ≥10 foci, n 250 cells per condition.

FIGS. ZS7A-ZS7B. Supplementary cell cycle analysis data. (FIG. ZS7A) Time course analysis of cell cycle distribution measured using integrated nuclear (Hoechst) staining intensity after treatment of LN229 MGMT+/−, MMR+/− cells with KL-50 (4a; 20 μM) or TMZ (1a; 20 μM) for 2, 8, 24, or 48 h. DMSO (0.1%) serves as negative control and aphidicolin (10 μM) and paclitaxel (1 μM) serve as positive controls for S-phase and G2-phase arrest, respectively. Columns, mean; error bars, SD; n=3 independent analyses. (FIG. ZS7B) Representative histograms showing DNA content distribution from 24 h and 48 h treatment conditions as quantified in (FIG. ZS7A).

FIGS. ZS8A-ZS8F. KL-50 (4a) induces DDR foci formation primarily in S and G2 cell cycle phases, and to lesser extent, in MGMT− G1 phase cells. (FIG. ZS8A and FIG. ZS8B) Phospho-SER139-H2AX (γH2AX) foci levels in LN229 MGMT+/−, MMR+/− cells in GI, S, and G2 cell cycle phases after treatment with 0.10% DMSO control, KL-50 (4a; 20 M) or TMZ (1a; 20 μM) for 48 h. Representative foci images with nuclei labeled as GI, S, or G2 phase cells based on Hoechst staining intensity are shown on the right. (FIG. ZS8C and FIG. ZS8D) 53BP1 foci levels and representative foci images in cells treated as in FIG. ZS8A and FIG. ZS8B. (FIG. ZS8E and FIG. ZS8F) Phospho-SER33-RPA2 (pRPA) foci levels and representative foci images in cells treated as in FIG. ZS8A and FIG. ZS8B. For FIGS. ZS8A-ZS8F, points, % cells with 10 foci; n≥500 cells per condition and cell cycle phase.

FIGS. ZS9A-ZS9G. Validation of micronuclei analysis, ICL sensitivity in FANCD2−/− and BRCA2−/− cell models, and demonstration of FANCD2 ubiquitination induced by KL-50 (4a). (FIG. ZS9A) Representative images of micronuclei identification. (FIG. ZS9B) Validation of micronuclei identification using olaparib as positive control. Change in percent cells with 1 micronuclei from baseline (DMSO control) after treatment with olaparib (10 M) for 48 h in LN229 MGMT+/−, MMR+/− cells. Columns, mean; error bars, SD; n 15 technical replicates; **** p<0.0001. (FIG. ZS9C) Western blotting performed in PD20 cells complemented with empty vector (EV), wildtype FANCD2 (WT), or ubiquitination-mutant FANCD2 (KR), demonstrating loss of MGMT in PD20 cells and comparable expression of MMR proteins. Western blotting in PEO1 BRCA2−/− and PEO4 BRCA2+ cells demonstrates intact expression of MGMT and MMR proteins. (FIG. ZS9D) Short-term viability assay curves for cisplatin and mitomycin (MMC) in PD20 cells, deficient in FANCD2 (FANCD2−/−) or complemented with FANCD2 (+FANCD2), demonstrating hypersensitivity to crosslinking agents in FANCD2−/− cells. (FIG. ZS9E) Short-term viability assay curves for cisplatin and MMC in PEO4 (BRCA2+) and PEO1 (BRCA2−/−) cells pre-treated with 0.01% DMSO control or 10 μM O⁶BG (+O⁶BG) for 1 h prior to cisplatin or MMC addition, demonstrating hypersensitivity of PEO4 BRCA2−/− cells to crosslinking agents independent of MGMT depletion. (FIG. ZS9F) Short-term viability assay curves for cisplatin and MMC in DLD1 BRCA2+/− and BRCA2−/− cells pre-treated with 0.01% DMSO control or 10 μM O⁶BG (+O⁶BG) for 1 h prior to cisplatin or MMC addition, demonstrating hypersensitivity of DLD1 BRCA2−/− cells to crosslinking agents independent of MGMT depletion. (FIG. ZS9G) Western blot analysis of FANCD2 ubiquitination in LN229 MGMT+/−, MMR+/− cells and PD20 FANCD2-deficient cells, complemented with empty vector (FANCD2+EV), wildtype FANCD2 (PD20+FD2) or ubiquitination-mutant FANCD2 (PD20+KR). The % FANCD2 ubiquitination (% FANCD2 Ub.) is quantified as the background-corrected integrated band intensity of the upper band divided by the sum of the background-corrected integrated band intensities of the upper and lower bands. The fold change in % FANCD2 ubiquitination is presented for each cell line relative to DMSO-treated cells. Vinculin serves as loading control. For (FIG. ZS9D), (FIG. ZS9E), and (FIG. ZS9F), points, mean; error bars, SD; n=3 technical replicates.

FIGS. ZS10A-Z10E. Spider plots tracking individual mouse tumor response to treatment. (FIG. ZS10A) Spider plots tracking LN229 MGMT−/MMR+ flank tumor volume of each mouse in response to treatment with P.O. 10% cyclodextrin vehicle control, TMZ (1a, 5 mg/kg MWF×3 weeks), or KL-50 (4a, 5 mg/kg MWF×3 weeks). (FIG. ZS10B) Spider plots tracking LN229 MGMT−/MMR− flank tumor volume of each mouse in response to treatment with PO 10% cyclodextrin vehicle control, TMZ (1a, 5 mg/kg MWF×3 weeks), or KL-50 (4a, 5 mg/kg MWF×3 weeks). (FIG. ZS10C and FIG. ZS10D) Spider plots tracking LN229 MGMT−/MMR+ and LN229 MGMT−/MMR− flank tumor volume in response to treatment with P.O 10% cyclodextrin control, P.O KL-50 (4a, 15 mg/kg MWF×3 weeks), P.O KL-50 (4a, 25 mg/kg M-F×1 week), or I.P. KL-50 (4a, 5 mg/kg MWF×3 weeks). (FIG. ZS10E) Spider plots tracking LN229 MGMT−/MMR− intracranial tumor size as measured by relative light units (photons/sec) in response to P.O treatment with 10% cyclodextrin vehicle control, TMZ (Ta, 25 mg/kg M-F×1 week), or KL-50 (4a, 25 mg/kg M-F×1 week).

### DETAILED DESCRIPTION OF THE DISCLOSURE

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

The present disclosure relates to compounds or agents for treating, ameliorating, and/or preventing cancer in a patient in need thereof. In one aspect, the compounds are a compound of Formula I. In another aspect, the compounds or agents comprise compounds of formula II, Ia, Ib, or Ic, and selectively treat cancer cells that have altered MGMT activity.

### Definitions

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise. The term “about” as used herein can allow for a degree of variability in a value or range, for example, within 10%, within 5%, or within 1% of a stated value or of a stated limit of a range, and includes the exact stated value or range. As used herein, “about” means plus or minus 10%.

As used herein, “optional” or “optionally” means that the subsequently described event or circumstance may or may not occur, and that the description includes instances where said event or circumstance occurs and instances where it does not.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)=CH₂, —C(CH₃)═CH(CH₃), —C(CH₂CH₃)=CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

As used herein, “lower alkyl” means a linear or branched saturated hydrocarbon of 1 to 6 carbon atoms, including methyl, ethyl, propyl, isopropyl, butyl, 2-methylpropyl, tert-butyl, and pentyl. In certain embodiments, “lower alkyl” refers to C₁-C₆ alkyl.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbomyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like. The term “deuteroalkyl” refers to an alkyl substituted by one or more deuterium. Examples of deuteroalky include —CD₃, —CHD₂, CH₂CD₃, and the like.

The term “heteroaralkynyl” as used herein refers to alkynyl groups as defined herein in which a hydrogen or carbon bond of an alkynyl group is replaced with a bond to a heteroaryl group as defined herein. Representative aralkynyl groups include, but are not limited to, 2-ethynylpyridine and 2-ethynylthiophene.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C2-heteroaryl can be a 5-ring with two carbon atoms and three heteroatoms, a 6-ring with two carbon atoms and four heteroatoms and so forth. Likewise a C4-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C2-heterocyclyl can be a 5-ring with two carbon atoms and three heteroatoms, a 6-ring with two carbon atoms and four heteroatoms and so forth. Likewise a C4-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

As used herein, the terms “individual”, “patient”, or “subject” can be used interchangeably and may refer to an individual organism, a vertebrate, a mammal (e.g., a bovine, a canine, a feline, or an equine), or a human. In a preferred embodiment, the individual, patient, or subject is a human.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The term “glioma” as used herein refers to a common type of tumor originating in the brain. About 33 percent of all brain tumors are gliomas, which originate in the glial cells that surround and support neurons in the brain, including astrocytes, oligodendrocytes and ependymal cells.

As used herein the term “MGMT deficient” (or MGMT⁻) cancers means cancers that have more than one standard deviation lower abundance of the mRNA transcript for the MGMT gene normalized to the relevant healthy control tissue. This MGMT deficiency can occur through promoter methylation, mutations in the gene, or through other methods resulting in downregulation of the gene.

As used herein, the term “MMR deficient” (or MMR⁻) cancers means cancers that have more than one standard deviation lower abundance of the mRNA transcript for any of the MMR genes (MSH2, MSH6, MLH1, MLH3, PMS2, PMS1) normalized to the relevant healthy control tissue. Alternatively, cancers that exhibit the microsatellite instability high phenotype (MSI-H) are also considered to be MMR deficient. See, for example, Li et al.—Microsatellite instability: a review of what the oncologist should know—Cancer Cell International, Article Number 16 (2020).

The term “knockdown” or “KD” as used herein refers to an experimental technique wherein the expression of one or more of an organisms genes and/or translation of the corresponding RNA is reduced.

As used herein, a “prophylactic” or “preventive” treatment is a treatment administered to a subject who does not exhibit signs of a disease or disorder or exhibits only early signs of the disease or disorder for the purpose of decreasing the risk of developing pathology associated with the disease or disorder.

As used herein, the phrases “pharmaceutically effective amount,” “therapeutically effective amount,” and “therapeutic level” mean a compound dose or plasma concentration in a subject, respectively, that provides the specific pharmacological effect for which the compound is administered in a subject in need of such treatment, i.e., to reduce, ameliorate, or eliminate the effects or symptoms of a disease or a disorder (for example, cancer). The desired treatment may be prophylactic and/or therapeutic. It is emphasized that a therapeutically effective amount or therapeutic level of a drug will not always be effective in treating the conditions/diseases described herein, even though such dosage is deemed to be a therapeutically effective amount by those of skill in the art. The therapeutically effective amount may vary based on the route of administration and dosage form, the age and weight of the subject, and/or the subject's condition, including the type and stage of the cancer at the time that treatment commences, among other factors. An appropriate therapeutic amount in any individual case may be determined by one of ordinary skill in the art using routine experimentation.

A “therapeutic response” means an improvement in at least one measure of cancer.

The terms “treatment” or “treating” as used herein with reference to a disease or disorder (for example, cancer) refer to reducing, ameliorating or eliminating one or more symptoms or effects of the disease or condition. The terms encompass reducing the severity of a symptom of a disease or disorder and/or the frequency of a symptom of a disease or disorder.

The term “prevent,” “preventing,” or “prevention” as used herein, comprises the prevention of at least one symptom associated with or caused by the state, disease or disorder being prevented.

The term “synergy” as used herein refers to the interaction or cooperation of two or more organizations, substances, or other agents to produce a combined effect greater than the sum of their separate effects. In on aspect, the term synergy may be applied the effect of a combination of agents for the treatment, prevention, and/or amelioration of disease and/or promotion or inhibition of a causal mechanism thereof.

As used herein, the term “refractory” as to a particular treatment of a disease means that the disease is unresponsive to the treatment.

As used herein, the term “pharmaceutical composition” or “composition” refers to a mixture of at least one compound useful within the invention with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically-acceptable carrier” means a material for admixture with a pharmaceutical compound (e.g., a chimeric compound) for administration to a patient as described, for example, in “Ansel's Pharmaceutical Dosage Forms and Delivery Systems”, Tenth Edition (2014). A “pharmaceutically acceptable carrier” refers to a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

The term “room temperature” as used herein refers to a temperature of about 15° C. to about 28° C.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

### I. Non-Limiting Overview of Mechanism and Structures

In certain embodiments, the strategy outlined in FIG. Z1A can be deployed to develop agents that overcome the resistance associated with MMR loss while maintaining TMZ's (1a) selectivity for MGMT-silenced tumors. These agents would deposit a primary lesion susceptible to S_{N}2-mediated removal by MGMT that could undergo a further chemical transformation to a secondary lesion capable of killing MGMT-deficient tumor cells in an MMR-independent manner. To maintain the therapeutic index between MGMT− tumor cells and MGMT+ healthy cells, the primary legion must undergo MGMT-mediated repair faster than it undergoes transformation to the secondary lesion. With these considerations in mind, it was hypothesized that an agent capable of depositing a 2-fluoroethyl lesion at O⁶-guanine would prove ideal as O⁶-(2-fluoroethyl)guanosine (S1) is known to hydrolyze slowly to N1-(2-hydroxyethyl)guanosine (S3) with a half-life of 18.5 h (37° C., pH 7.4) (FIG. ZS1A). Mechanistically, this occurs via N1 displacement of the pendent fluoride to provide the N1,O⁶-ethanoguanosine intermediate S2 which undergoes ring-opening nucleophilic attack by water to give S3. By analogy, the G(N1)-C(N3) interstrand cross-link (ICL) 8 may form by conversion of O⁶FEtG (5) to the N1,O⁶-ethanoguanine intermediate 6 followed by ring-opening by N3 of the complementary cytosine base (7; FIG. Z1E). As MGMT reacts rapidly with alkylated DNA (a second-order rate constant of 1×10⁹ M⁻¹·min⁻¹ was measured using methylated calf thymus DNA as substrate) and can act upon a wide range of O⁶-alkylguanine substrates, MGMT-proficient cells should repair the O⁶FEtG lesion (5) before it transforms into ICL 8.

### II. Compounds

In one aspect, the present disclosure provides a compound of formula (I) or a salt thereof:

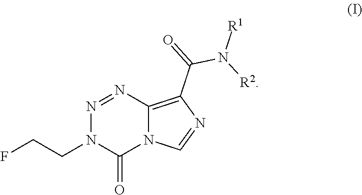

In certain embodiments, R¹ and R² are each independently selected from H and lower alkyl. In certain embodiments, R¹ and R² combine to form —(CH₂)ₙ—. In certain embodiments, n is 2, 3, 4, or 5. In certain embodiments, R and R² are not simultaneously H.

In certain embodiments, R and R² are each independently selected from H and lower alkyl; or R¹ and R² combine to form —(CH₂)ₙ—; and n is 2, 3, 4, or 5; provided R¹ and R² are not simultaneously H.

In certain embodiments, the compound is a compound of formula (I).

In certain embodiments, R¹ is H. In certain embodiments, R¹ is lower alkyl. In certain embodiments, R² is H. In certain embodiments, R² is lower alkyl. In certain embodiments, R¹ is H, and R² is lower alkyl. In certain embodiments, R¹ and R² are each independently lower alkyl. In certain embodiments, R¹ and R² are each methyl.

In certain embodiments, R¹ is selected from the group consisting of methyl, ethyl, propyl, isopropyl, butyl, 2-methylpropyl, tert-butyl, pentyl, and hexyl.

In certain embodiments, R¹ is methyl. In certain embodiments, R¹ is ethyl. In certain embodiments, R¹ is propyl. In certain embodiments, R¹ is isopropyl. In certain embodiments, R¹ is butyl. In certain embodiments, R¹ is 2-methylpropyl. In certain embodiments, R¹ is tert-butyl. In certain embodiments, R¹ is pentyl. In certain embodiments, R¹ is hexyl.

In certain embodiments, R² is selected from the group consisting of methyl, ethyl, propyl, isopropyl, butyl, 2-methylpropyl, tert-butyl, pentyl, and hexyl.

In certain embodiments, R² is methyl. In certain embodiments, R² is ethyl. In certain embodiments, R² is propyl. In certain embodiments, R² is isopropyl. In certain embodiments, R² is butyl. In certain embodiments, R² is 2-methylpropyl. In certain embodiments, R² is tert-butyl. In certain embodiments, R² is pentyl. In certain embodiments, R² is hexyl.

In certain embodiments, R¹ and R² combine to form —(CH₂)ₙ—. In certain embodiments, n is 2. In certain embodiments, n is 3. In certain embodiments, n is 4. In certain embodiments, n is 5.

In certain embodiments, the compound is

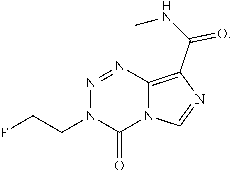 [SMILES: CNC(=O)c1ncn2c(=O)n(CCF)nnc12]

In certain embodiments, the compound is

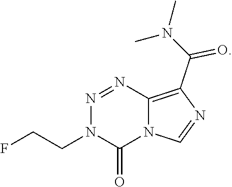 [SMILES: CN(C)C(=O)c1ncn2c(=O)n(CCF)nnc12]

In certain embodiments, the compound is

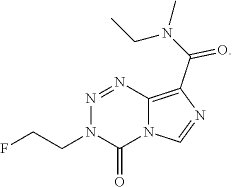 [SMILES: CCN(C)C(=O)c1ncn2c(=O)n(CCF)nnc12]

In certain embodiments, the compound is

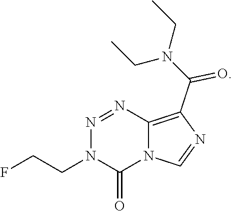 [SMILES: CCN(CC)C(=O)c1ncn2c(=O)n(CCF)nnc12]

In certain embodiments, the compound is

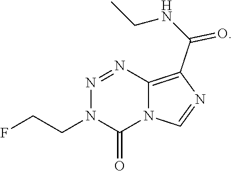 [SMILES: CCNC(=O)c1ncn2c(=O)n(CCF)nnc12]

In certain embodiments, the compound is

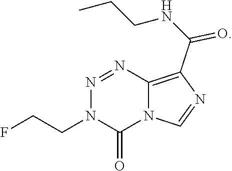 [SMILES: CCCNC(=O)c1ncn2c(=O)n(CCF)nnc12]

In certain embodiments, the compound is

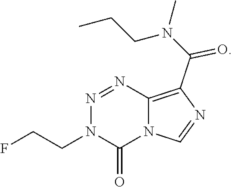 [SMILES: CCCN(C)C(=O)c1ncn2c(=O)n(CCF)nnc12]

In certain embodiments, the compound is

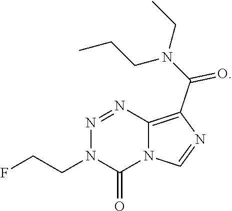 [SMILES: CCCN(CC)C(=O)c1ncn2c(=O)n(CCF)nnc12]

In certain embodiments, the compound is

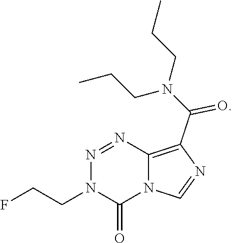 [SMILES: CCCN(CCC)C(=O)c1ncn2c(=O)n(CCF)nnc12]

In certain embodiments, the compound is

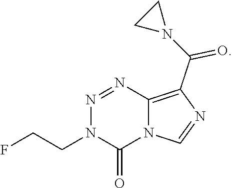 [SMILES: O=C(c1ncn2c(=O)n(CCF)nnc12)N1CC1]

In certain embodiments, the compound is

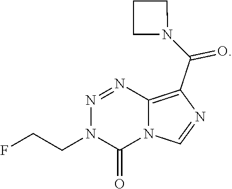 [SMILES: O=C(c1ncn2c(=O)n(CCF)nnc12)N1CCC1]

In certain embodiments, the compound is

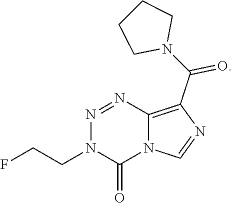 [SMILES: O=C(c1ncn2c(=O)n(CCF)nnc12)N1CCCC1]

In certain embodiments, the compound is

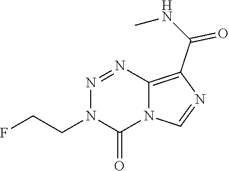 [SMILES: CNC(=O)c1ncn2c(=O)n(CCF)nnc12]

or a pharmaceutically acceptable salt thereof.

In certain embodiments, the compound is

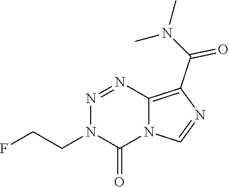 [SMILES: CN(C)C(=O)c1ncn2c(=O)n(CCF)nnc12]

or a pharmaceutically acceptable salt thereof.

In certain embodiments, the compound is

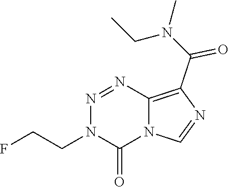 [SMILES: CCN(C)C(=O)c1ncn2c(=O)n(CCF)nnc12]

or a pharmaceutically acceptable salt thereof.

In certain embodiments, the compound is

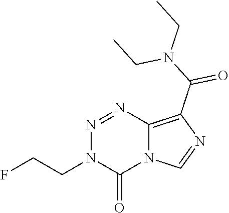 [SMILES: CCN(CC)C(=O)c1ncn2c(=O)n(CCF)nnc12]

or a pharmaceutically acceptable salt thereof.

In certain embodiments, the compound is

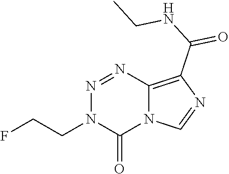 [SMILES: CCNC(=O)c1ncn2c(=O)n(CCF)nnc12]

or a pharmaceutically acceptable salt thereof.

In certain embodiments, the compound is

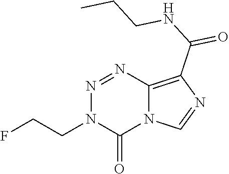 [SMILES: CCCNC(=O)c1ncn2c(=O)n(CCF)nnc12]

or a pharmaceutically acceptable salt thereof.

In certain embodiments, the compound is

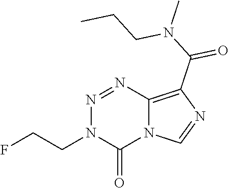 [SMILES: CCCN(C)C(=O)c1ncn2c(=O)n(CCF)nnc12]

or a pharmaceutically acceptable salt thereof.

In certain embodiments, the compound is

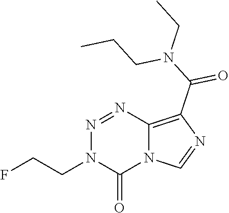 [SMILES: CCCN(CC)C(=O)c1ncn2c(=O)n(CCF)nnc12]

or a pharmaceutically acceptable salt thereof.

In certain embodiments, the compound is

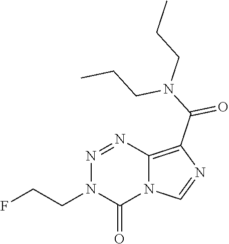 [SMILES: CCCN(CCC)C(=O)c1ncn2c(=O)n(CCF)nnc12]

or a pharmaceutically acceptable salt thereof.

In certain embodiments, the compound is

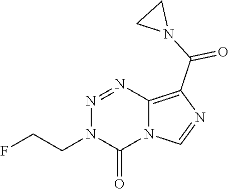 [SMILES: O=C(c1ncn2c(=O)n(CCF)nnc12)N1CC1]

or a pharmaceutically acceptable salt thereof.

In certain embodiments, the compound is

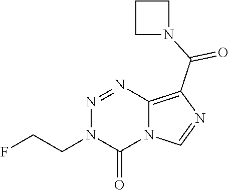 [SMILES: O=C(c1ncn2c(=O)n(CCF)nnc12)N1CCC1]

or a pharmaceutically acceptable salt thereof.

In certain embodiments, the compound is

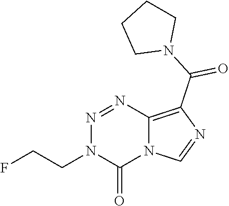 [SMILES: O=C(c1ncn2c(=O)n(CCF)nnc12)N1CCCC1]

or a pharmaceutically acceptable salt thereof.

In another aspect, the present disclosure provides a compound of formula (II) or a salt thereof:

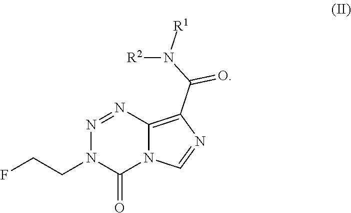

In certain embodiments, R¹ is selected from H and lower alkyl. In certain embodiments, R² is selected from H, lower alkyl, trifluoroethyl,

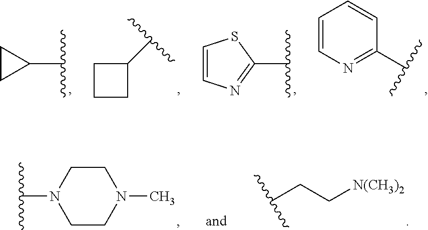

In certain embodiments, R¹ is H when R² is other than H or lower alkyl. In certain embodiments, R¹ and R² may combine to form —(CH₂)ₙ— or —(CH₂)₂—N(CH₃)—(CH₂)₂—. In certain embodiments, n is 3, 4, or 5. In certain embodiments, R¹ and R² are not both H.

In certain embodiments, R¹ is selected from H and lower alkyl; R² is selected from H, lower alkyl, trifluoroethyl,

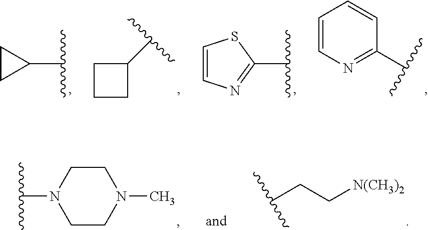

provided that R¹ is H when R² is other than H or lower alkyl; or R¹ and R² may combine to form —(CH₂)ₙ— or —(CH₂)₂—N(CH₃)—(CH₂)₂—; and n is 3, 4, or 5; provided that R¹ and R² are not both H.

In certain embodiments, the compound is a compound of formula (II).

In certain embodiments, R¹ is H. In certain embodiments, R¹ is lower alkyl.

In certain embodiments, R² is selected from H, lower alkyl, trifluoroethyl,

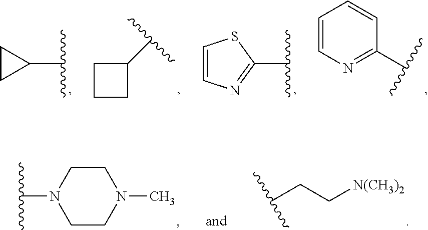

In certain embodiments, R² is H. In certain embodiments, R² is lower alkyl. In certain embodiments, R² is trifluoroethyl. In certain embodiments, R² is

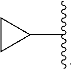 [SMILES: *C1CC1]

In certain embodiments, R² is

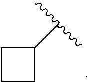 [SMILES: *C1CCC1]

In certain embodiments, R² is

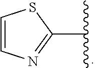 [SMILES: *c1nccs1]

In certain embodiments, R² is

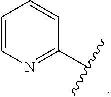 [SMILES: *c1ccccn1]

In certain embodiments, R² is

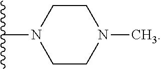 [SMILES: *N1CCN(C)CC1]

In certain embodiments, R² is

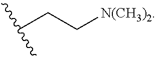 [SMILES: *CCN(C)C]

In certain embodiments, R¹ is H, and R² is trifluoroethyl,

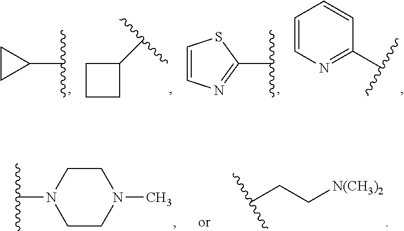

In certain embodiments, R¹ is selected from the group consisting of methyl, ethyl, propyl, isopropyl, butyl, 2-methylpropyl, tert-butyl, pentyl, and hexyl.

In certain embodiments, R² is selected from the group consisting of methyl, ethyl, propyl, isopropyl, butyl, 2-methylpropyl, tert-butyl, pentyl, and hexyl.

In certain embodiments, R¹ is methyl. In certain embodiments, R¹ is ethyl. In certain embodiments, R¹ is propyl. In certain embodiments, R¹ is isopropyl. In certain embodiments, R¹ is butyl. In certain embodiments, R¹ is 2-methylpropyl. In certain embodiments, R¹ is tert-butyl. In certain embodiments, R¹ is pentyl. In certain embodiments, R¹ is hexyl.

In certain embodiments, R² is methyl. In certain embodiments, R² is ethyl. In certain embodiments, R² is propyl. In certain embodiments, R² is isopropyl. In certain embodiments, R² is butyl. In certain embodiments, R² is 2-methylpropyl. In certain embodiments, R² is tert-butyl. In certain embodiments, R² is pentyl. In certain embodiments, R² is hexyl.

In certain embodiments, R¹ and R² combine to form —(CH₂)ₙ— or —(CH₂)₂—N(CH₃)—(CH₂)₂—. In certain embodiments, R¹ and R² combine to form —(CH₂)ₙ—. In certain embodiments, R¹ and R² combine to form —(CH₂)₂—N(CH₃)—(CH₂)₂—.

In certain embodiments, n is 3. In certain embodiments, n is 4. In certain embodiments, n is 5.

In certain embodiments, the compound is

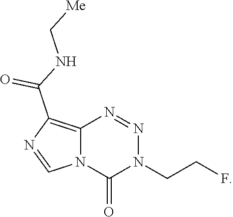 [SMILES: CCNC(=O)c1ncn2c(=O)n(CCF)nnc12]

In certain embodiments, the compound is

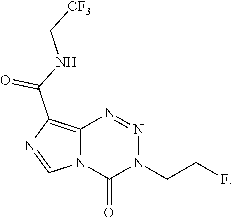 [SMILES: O=C(NCC(F)(F)F)c1ncn2c(=O)n(CCF)nnc12]

In certain embodiments, the compound is

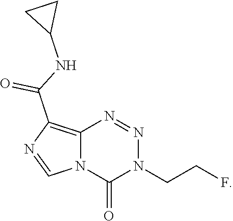 [SMILES: O=C(NC1CC1)c1ncn2c(=O)n(CCF)nnc12]

In certain embodiments, the compound is

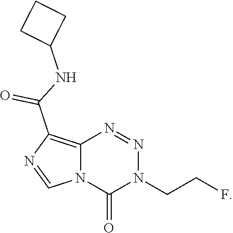 [SMILES: O=C(NC1CCC1)c1ncn2c(=O)n(CCF)nnc12]

In certain embodiments, the compound is

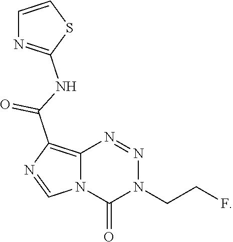 [SMILES: O=C(Nc1nccs1)c1ncn2c(=O)n(CCF)nnc12]

In certain embodiments, the compound is

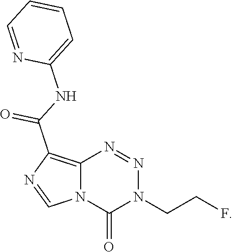 [SMILES: O=C(Nc1ccccn1)c1ncn2c(=O)n(CCF)nnc12]

In certain embodiments, the compound is

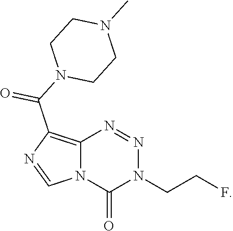 [SMILES: CN1CCN(C(=O)c2ncn3c(=O)n(CCF)nnc23)CC1]

In certain embodiments, the compound is

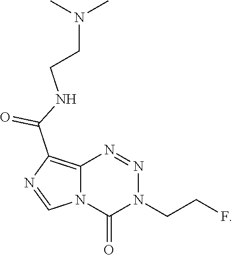 [SMILES: CN(C)CCNC(=O)c1ncn2c(=O)n(CCF)nnc12]

In certain embodiments, the compound is

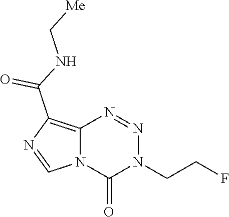 [SMILES: CCNC(=O)c1ncn2c(=O)n(CCF)nnc12]

or a pharmaceutically acceptable salt thereof.

In certain embodiments, the compound is

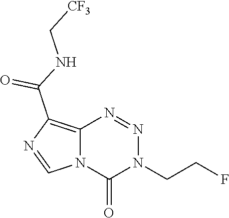 [SMILES: O=C(NCC(F)(F)F)c1ncn2c(=O)n(CCF)nnc12]

or a pharmaceutically acceptable salt thereof.

In certain embodiments, the compound is

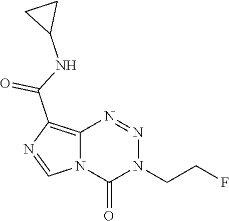 [SMILES: O=C(NC1CC1)c1ncn2c(=O)n(CCF)nnc12]

or a pharmaceutically acceptable salt thereof.

In certain embodiments, the compound is

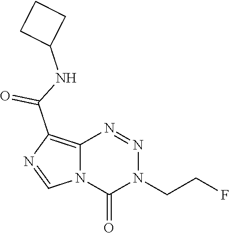 [SMILES: O=C(NC1CCC1)c1ncn2c(=O)n(CCF)nnc12]

or a pharmaceutically acceptable salt thereof.

In certain embodiments, the compound is

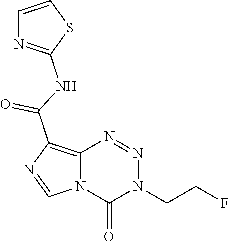 [SMILES: O=C(Nc1nccs1)c1ncn2c(=O)n(CCF)nnc12]

or a pharmaceutically acceptable salt thereof.

In certain embodiments, the compound is

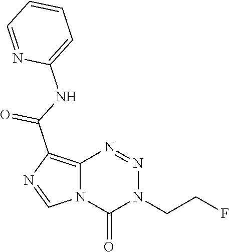 [SMILES: O=C(Nc1ccccn1)c1ncn2c(=O)n(CCF)nnc12]

or a pharmaceutically acceptable salt thereof.

In certain embodiments, the compound is

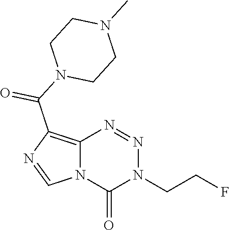 [SMILES: CN1CCN(C(=O)c2ncn3c(=O)n(CCF)nnc23)CC1]

or a pharmaceutically acceptable salt thereof.

In certain embodiments, the compound is

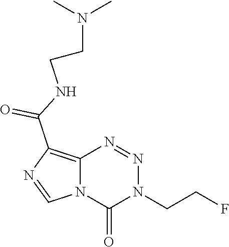 [SMILES: CN(C)CCNC(=O)c1ncn2c(=O)n(CCF)nnc12]

or a pharmaceutically acceptable salt thereof.

In another aspect, the present disclosure provides a compound of formula (IIA), or a pharmaceutically acceptable salt thereof:

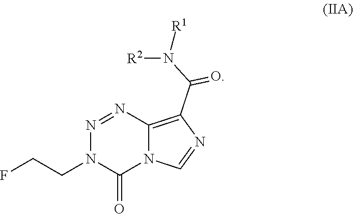

In certain embodiments, R¹ is selected from H and C₁₋₄ deuteroalkyl. In certain embodiments, R² is selected from H, C₁₋₄ alkyl, benzyl, trifluoromethyl,

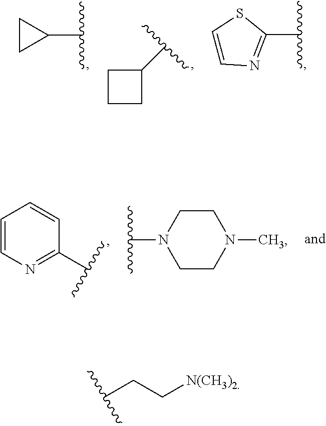

In certain embodiments, R¹ and R² are not both H.

In certain embodiments, R¹ is selected from H and C₁₋₄ deuteroalkyl; R² is selected from H, C₁₋₄ alkyl, benzyl, trifluoromethyl,

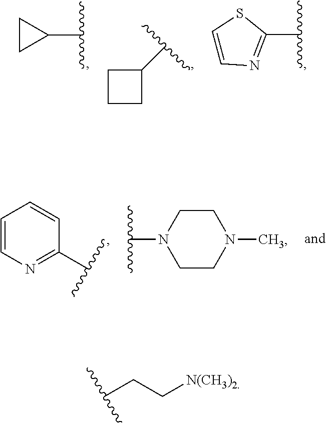

provided that R¹ and R² are not both H.

In certain embodiments, the compound is a compound of formula (IIA).

In certain embodiments, R¹ is H. In certain embodiments, R¹ is C₁₋₄ deuteroalkyl. In certain embodiments, R¹ is CD₃.

In certain embodiments, when R¹ is a C₁₋₄ deuteroalkyl, any number of hydrogen atoms (H) can be replaced by deuterium atoms (D), and all deuterated isomers in the C₁₋₄ deuteroalkyl are contemplated. For example, and without limitation, CH₂D, CHD₂, CD₃, CH₂CH₂D, CH₂CHD₂, CH₂CD₃, CHDCH₃, and the like, and all analogous deuteroalkyl groups are contemplated.

In certain embodiments, R² is selected from H, C₁₋₄ alkyl, benzyl trifluoromethyl,

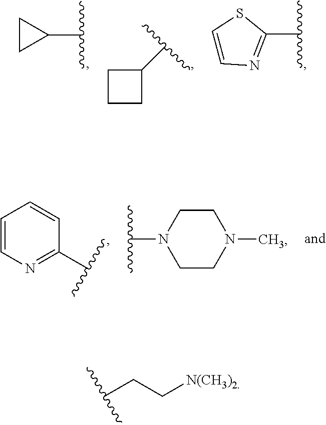

In certain embodiments, R² is H. In certain embodiments, R² is C₁₋₄ alkyl. In certain embodiments, R² is benzyl. In certain embodiments, R² is trifluoromethyl. In certain embodiments, R² is

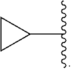 [SMILES: *C1CC1]

In certain embodiments, R² is

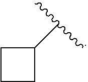 [SMILES: *C1CCC1]

In certain embodiments, R² is

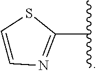 [SMILES: *c1nccs1]

In certain embodiments, R² is

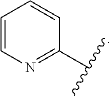 [SMILES: *c1ccccn1]

In certain embodiments, R² is

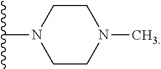 [SMILES: *N1CCN(C)CC1]

In certain embodiments, R² is

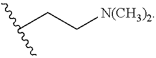 [SMILES: *CCN(C)C]

In certain embodiments, R is CD₃, and R² is benzyl, trifluoromethyl,

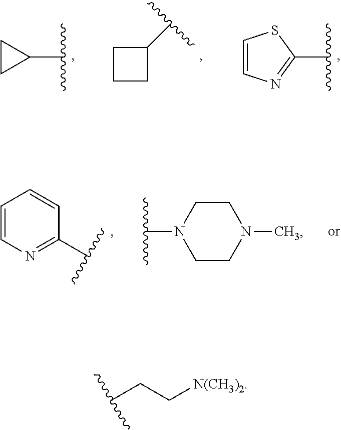

In certain embodiments, the compound is a compound in Table I-1, or a pharmaceutically acceptable salt thereof.

TABLE I-1

| No. | Chemical Structure |
|---|---|
| I-1 | 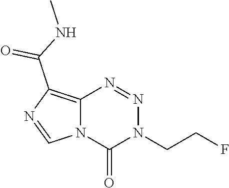 [SMILES: CNC(=O)c1ncn2c(=O)n(CCF)nnc12] |
| I-2 | 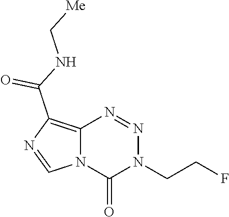 [SMILES: CCNC(=O)c1ncn2c(=O)n(CCF)nnc12] |
| I-3 | 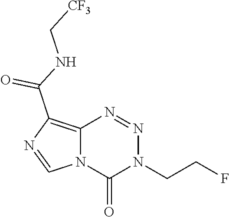 [SMILES: O=C(NCC(F)(F)F)c1ncn2c(=O)n(CCF)nnc12] |
| I-4 | 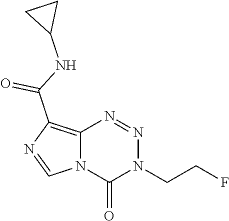 [SMILES: O=C(NC1CC1)c1ncn2c(=O)n(CCF)nnc12] |
| I-5 | 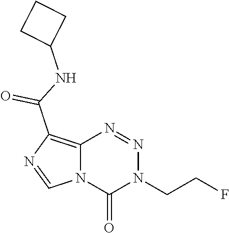 [SMILES: O=C(NC1CCC1)c1ncn2c(=O)n(CCF)nnc12] |
| I-6 | 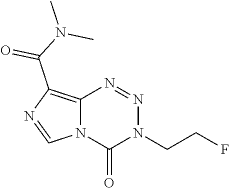 [SMILES: CN(C)C(=O)c1ncn2c(=O)n(CCF)nnc12] |
| I-7 | 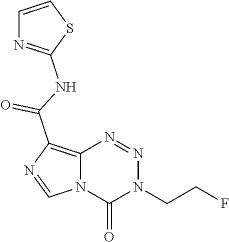 [SMILES: O=C(Nc1nccs1)c1ncn2c(=O)n(CCF)nnc12] |
| I-8 | 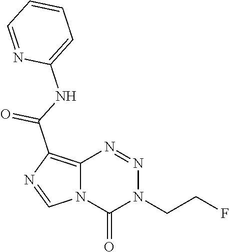 [SMILES: O=C(Nc1ccccn1)c1ncn2c(=O)n(CCF)nnc12] |
| I-9 | 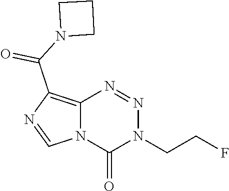 [SMILES: O=C(c1ncn2c(=O)n(CCF)nnc12)N1CCC1] |
| I-10 | 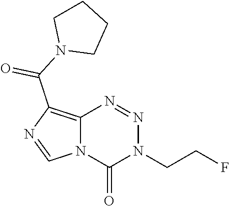 [SMILES: O=C(c1ncn2c(=O)n(CCF)nnc12)N1CCCC1] |
| I-11 | 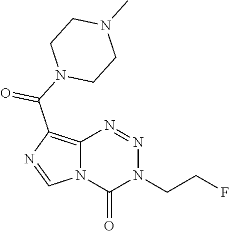 [SMILES: CN1CCN(C(=O)c2ncn3c(=O)n(CCF)nnc23)CC1] |
| I-12 | 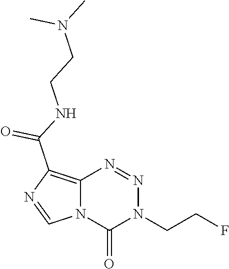 [SMILES: CN(C)CCNC(=O)c1ncn2c(=O)n(CCF)nnc12] |
| I-13 | 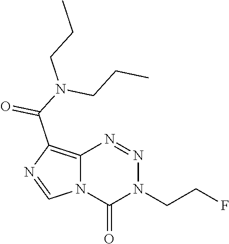 [SMILES: CCCN(CCC)C(=O)c1ncn2c(=O)n(CCF)nnc12] |
| I-14 | 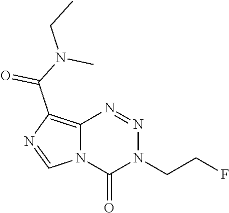 [SMILES: CCN(C)C(=O)c1ncn2c(=O)n(CCF)nnc12] |
| I-15 | 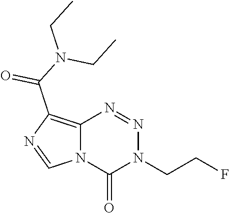 [SMILES: CCN(CC)C(=O)c1ncn2c(=O)n(CCF)nnc12] |
| I-16 | 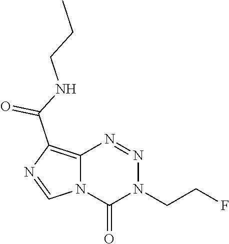 [SMILES: CCCNC(=O)c1ncn2c(=O)n(CCF)nnc12] |
| I-17 | 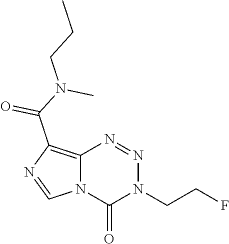 [SMILES: CCCN(C)C(=O)c1ncn2c(=O)n(CCF)nnc12] |
| I-18 | 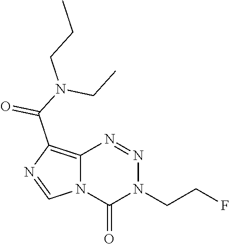 [SMILES: CCCN(CC)C(=O)c1ncn2c(=O)n(CCF)nnc12] |
| I-19 | 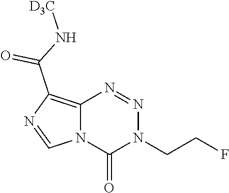 [SMILES: [2H]C([2H])([2H])NC(=O)c1ncn2c(=O)n(CCF)nnc12] |
| I-20 | 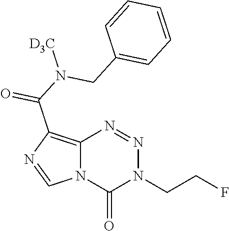 [SMILES: [2H]C([2H])([2H])N(Cc1ccccc1)C(=O)c1ncn2c(=O)n(CCF)nnc12] |
| I-21 | 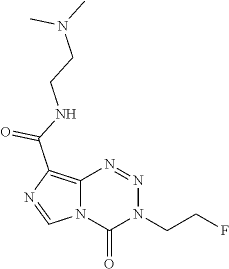 [SMILES: CN(C)CCNC(=O)c1ncn2c(=O)n(CCF)nnc12] |

In another aspect, the present disclosure provides a compound of formula (Ia-Ic), which is selected from the group consisting of:

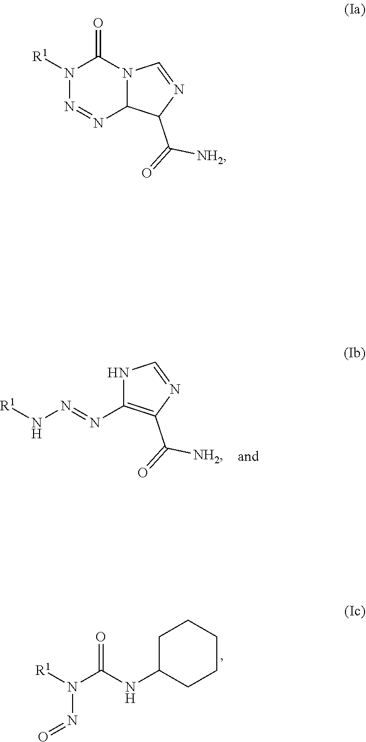

or pharmaceutically acceptable salts thereof. In certain embodiments, R¹ is selected from the group consisting of optionally substituted C₁-C₆ alkyl and optionally substituted C₁-C₆ haloalkyl. In certain embodiments, each optional substituent in R¹ is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl. In certain embodiments, R¹ is selected from the group consisting of optionally substituted C₁-C₆ alkyl and optionally substituted C₁-C₆ haloalkyl; wherein each optional substituent in R¹ is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl.

In certain embodiments, the compound is selected from the group consisting of

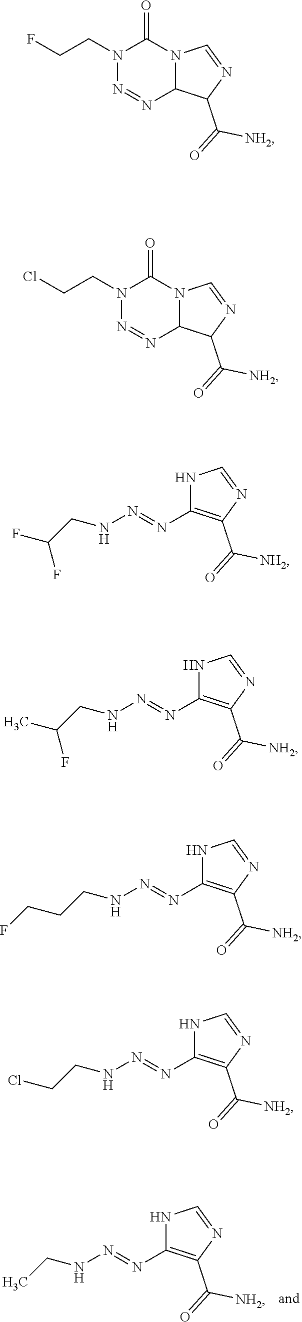

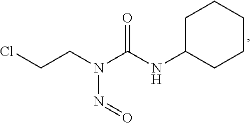 [SMILES: O=NN(CCCl)C(=O)NC1CCCCC1]

or pharmaceutically acceptable salts thereof.

In another aspect, the present disclosure provides a compound of formula (II) and the pharmaceutically acceptable salts thereof:

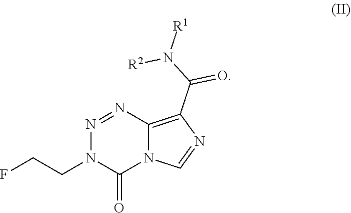

In certain embodiments, R¹ is individually selected from H and lower alkyl. In certain embodiments, R² is individually selected from H, lower alkyl, trifluoromethyl,

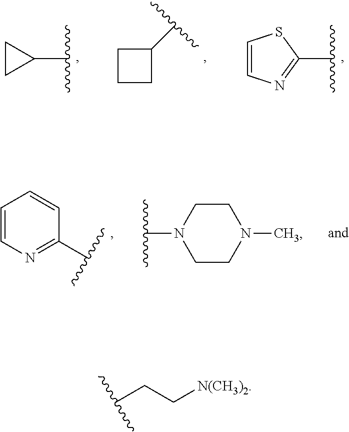

In certain embodiments, R¹ is H when R² is other than H or lower alkyl. In certain embodiments, R¹ and R² are not both H. In certain embodiments, R¹ and R² may combine to form —(CH₂)ₙ—. In certain embodiments, n is 3, 4, or 5. In certain embodiments, R¹ and R² may combine to form —(CH₂)₂—N(CH₃)—(CH₂)₂—.

In certain embodiments, R¹ is individually selected from H and lower alkyl, R² is individually selected from H, lower alkyl, trifluoromethyl,

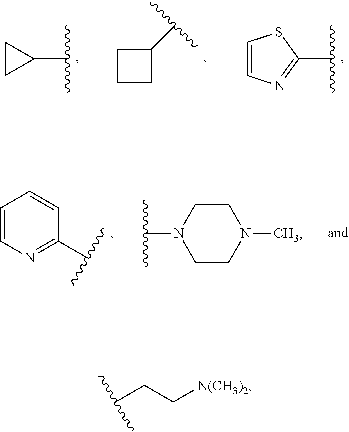

provided that R¹ is H when R² is other than H or lower alkyl and further provided that R¹ and R² are not both H, and R¹ and R² may combine to form —(CH₂)ₙ—, wherein n is 3, 4, or 5, or may combine to form —(CH₂)₂—N(CH₃)—(CH₂)₂—.

The compound of formula (II) wherein R¹ and R² are both H is sometimes referred to herein as “KL50” and the compound of formula (II) wherein R¹ is H and R² is methyl is sometimes referred to herein as “N-methyl KL50”.

The disclosure also provides certain novel compounds of formula (II), particularly those wherein R¹ and R² are not both H. These compounds are more potent anti-cancer compounds than temozolomide against MGMT deficient cancers regardless of MMR status, as well as being effective against MMR deficient cancers and cancers that are refractory to treatment by temozolomide.

In certain embodiments, the compound is at least one of the following:

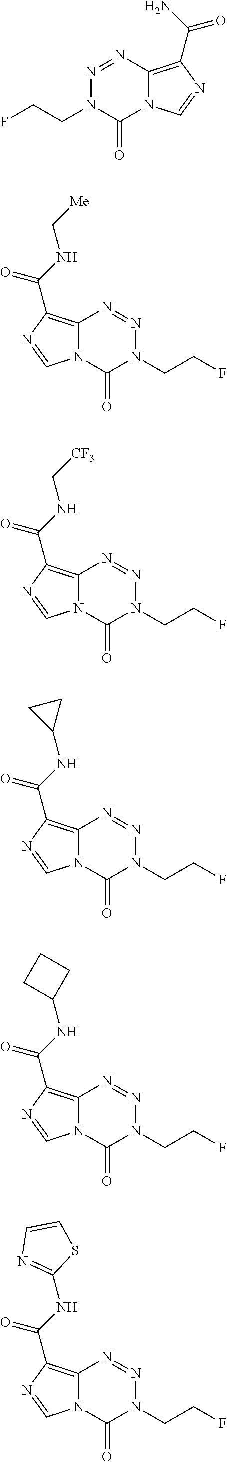 [SMILES: NC(=O)c1ncn2c(=O)n(CCF)nnc12 ; CCNC(=O)c1ncn2c(=O)n(CCF)nnc12 ; O=C(NCC(F)(F)F)c1ncn2c(=O)n(CCF)nnc12 ; O=C(NC1CC1)c1ncn2c(=O)n(CCF)nnc12 ; O=C(NC1CCC1)c1ncn2c(=O)n(CCF)nnc12 ; O=C(Nc1nccs1)c1ncn2c(=O)n(CCF)nnc12]

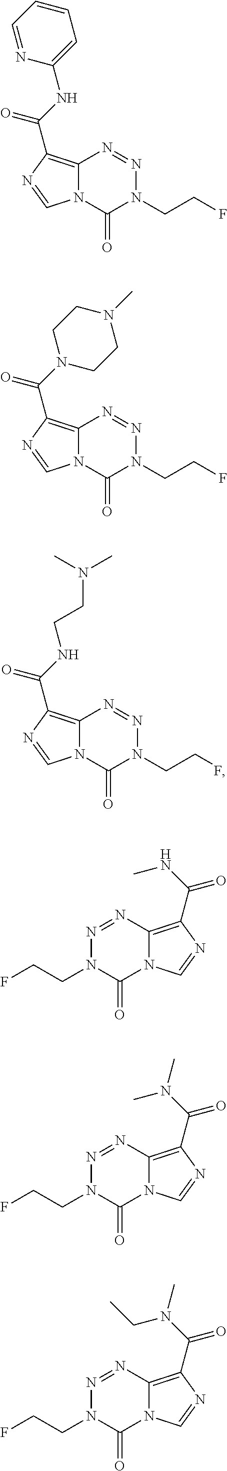

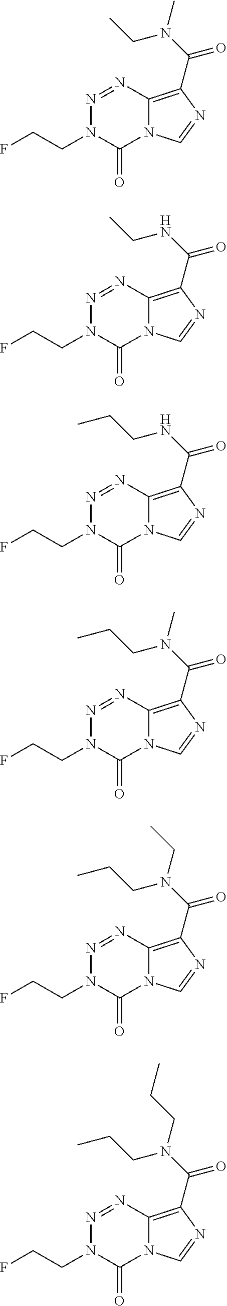

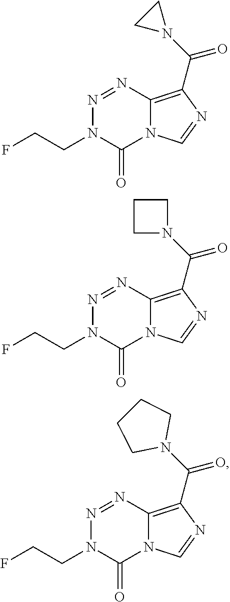

or pharmaceutically acceptable salts thereof.

In certain embodiments, the compound, or a pharmaceutically acceptable salt thereof, has the following structure:

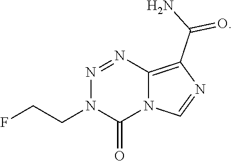 [SMILES: NC(=O)c1ncn2c(=O)n(CCF)nnc12]

The compounds are useful for the treatment, prevention, and/or amelioration of cancer, wherein the compounds induce DNA lesions in the cell that lead to irreparable DNA damage and/or unrepaired lesions.

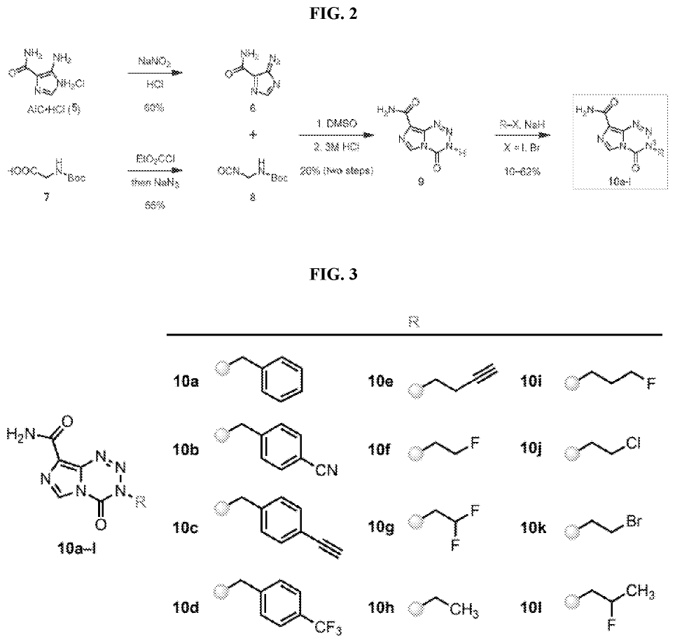

The compounds of formula (II) described in FIG. 3 were synthesized according to the process described in FIG. 2. The synthetic route up to nortemozolomide (compound 9) was carried out as described in Mosley et al. Org Lett, 14, 5872-5875 (2012). Nortemozolomide was dissolved in [0.08] M dry DMF at 0° C., followed by adding 1.05 equivalents of NaH in mineral oil. To the stirring solution was charged 2 equivalents of the electrophile (R—X) After five minutes. The reaction was allowed to proceed at room temperature for 12 hours, the solvent was removed, and the crude product was purified via column chromatography using 95:5 methylene chloride-methanol.

Compounds 10a-10l are shown in FIG. 3. Temozolomide (not shown) has R=methyl in the structure of FIG. 3.

The compounds of formula (II) that are described in FIG. 3, as well as the compounds of formula (II) that are not described in FIG. 3 may be synthesized by the following scheme:

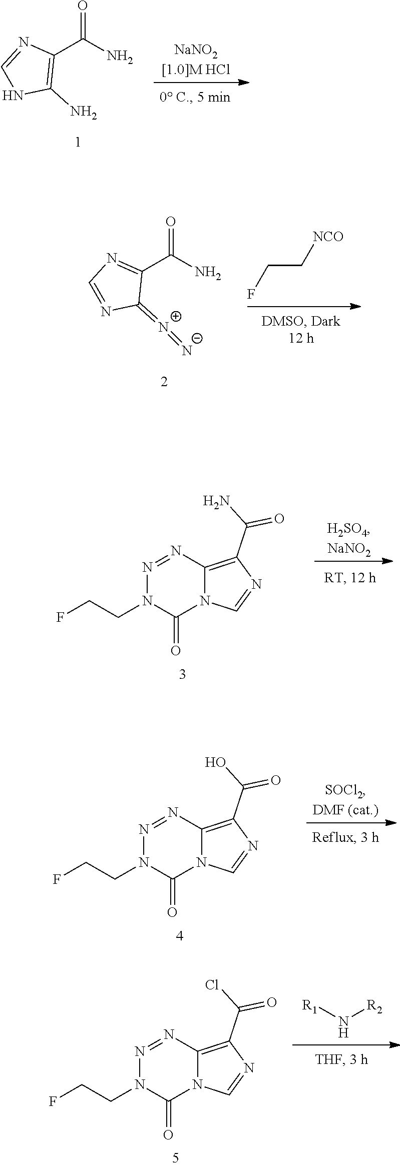

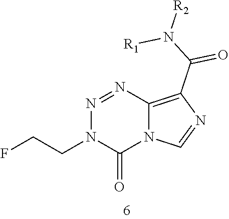

Synthesis of 2: To a stirring solution of NaNO₂ (10.47 g, 150.8 mmol) in 200 mL H2O at 0° C. was added a solution of 1 (17.3 g, 137.17 mmol) in 200 mL of 1 M HCl drop wise with magnetic stirring. A yellow precipitate quickly began to form and the mixture was allowed to stir at this temperature for 5 minutes once fully combined. The precipitate formed was then filtered off, washed with water, and dried via lyophilization.

Synthesis of 3: To a stirring solution of 2 (1 g, 7.3 mmol) in 12 mL DMSO was added 1.75 equivalents of 2-fluoroethyl isocyanate dropwise (1.14 g, 12.78 mmol) with magnetic stirring. The solution was allowed to stir in the dark for 12 hours. The DMSO was removed, and the compound purified via column chromatography with a methylene chloride:methanol gradient.

Synthesis of 4: To round bottom flask containing 3 (226 mg, 1 mmol) was added 1.56 mL of concentrated sulfuric acid with magnetic stirring. Then NaNO₂ (252 mg, 3.65 mmol) dissolved in 1 mL H2O was added dropwise to the reaction vessel and allowed to react at room temperature for 12 hours. Afterwards, a mixture of water and ice was added to quench the reaction which precipitated the product. The compound was then recovered by vacuum filtration.

Synthesis of 5 & 6: Compound 4 (50 mg, 0.22 mmol) was dissolved in 1 mL of thionyl chloride with 1 drop of DMF and brought to reflux for 3 hours. The compound was then concentrated by rotary evaporation to dryness to yield crude compound 5. To the crude 5 was added 10 mL of THF and 1.05 equivalents of the desired amine R¹R²NH, wherein R¹ and R² are as defined above. This mixture was allowed to react for 3 hours, then concentrated and purified via column chromatography using a hexane:EtOAc gradient.

Additional synthetic procedures are described in the Examples.

The present disclosure further provides a pharmaceutical composition comprising a compound of the present disclosure and at least one pharmaceutically acceptable carrier.

The compounds of the invention may possess one or more stereocenters, and each stereocenter may exist independently in either the (R)- or (S)-configuration. In certain embodiments, compounds described herein are present in optically active or racemic forms. The compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the invention exist as tautomers. All tautomers are included within the scope of the compounds recited herein.

In certain embodiments, compounds described herein are prepared as prodrugs. A “prodrug” is an agent converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds of the invention are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

### Salts

The compositions described herein may form salts with acids or bases, and such salts are included in the present invention. In certain embodiments, the salts are pharmaceutically acceptable salts. The term “salts” embraces addition salts of free acids or free bases that are compositions of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compositions of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compositions of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. Examples of pharmaceutically unacceptable base addition salts include lithium salts and cyanate salts. All of these salts may be prepared from the corresponding composition by reacting, for example, the appropriate acid or base with the composition.

### III. Therapeutic Methods

The present disclosure provides a method of treating, preventing, and/or ameliorating cancer in a subject in need thereof. In certain embodiments, the cancer is charactered by a cancer cell having altered MGMT activity. In certain embodiments, the method comprises administering to the subject an agent that induces DNA lesions in the cell that lead to irreparable DNA damage to selectively treat the cancer.

In one aspect, the disclosure provides a method of treating, preventing, and/or ameliorating cancer in a subject in need thereof, comprising administering to the subject a therapeutically effective amount of a compound described herein, such as a compound of formula (I).

In another aspect, the disclosure provides a method of treating, preventing, and/or ameliorating cancer in a subject in need thereof, comprising administering to the subject a therapeutically effective amount of a compound described herein (such as a compound of formula (I) or (II)), wherein the cancer is MGMT deficient and either MMR deficient or refractory to treatment with temozolomide.

In certain embodiments, the cancer is a solid tumor, leukemia, or lymphoma. In certain embodiments, the cancer is a solid tumor. In certain embodiments, the cancer is a brain tumor. In certain embodiments, the cancer is urothelial cancer, breast invasive carcinoma, colon adenocarcinoma, head and neck tumor (SCC), lung adenocarcinoma, rectum adenocarcinoma, acute myeloid leukemia, glioblastoma multiforme, or brain lower grade glioma.

In certain embodiments, the cancer is glioma, colorectal cancer, non-small cell lung cancer, small cell lung cancer, sarcoma, pancreatic cancer, neuroendocrine tumor, esophageal cancer, lymphoma, head & neck cancer, breast cancer, bladder cancer, or leukemia. In certain embodiments, the cancer is glioblastoma multiforme.

In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia.

In certain embodiments, the cancer is squamous cell cancer, lung cancer including small cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, and head and neck cancer. In certain embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML, lymphoma, leukemia, multiple myeloma myeloproliferative diseases, large B cell lymphoma, or B cell Lymphoma.

In certain embodiments, the cancer is a solid tumor or leukemia. In certain other embodiments, the cancer is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, esophagus cancer, liver cancer, an acoustic neuroma, oligodendroglioma, meningioma, neuroblastoma, or retinoblastoma. In certain other embodiments, the cancer is small cell lung cancer, non-small cell lung cancer, melanoma, cancer of the central nervous system tissue, brain cancer, Hodgkin's lymphoma, non-Hodgkin's lymphoma, cutaneous T-Cell lymphoma, cutaneous B-Cell lymphoma, or diffuse large B-Cell lymphoma. In certain other embodiments, the cancer is breast cancer, colon cancer, small-cell lung cancer, non-small cell lung cancer, prostate cancer, renal cancer, ovarian cancer, leukemia, melanoma, or cancer of the central nervous system tissue. In certain other embodiments, the cancer is colon cancer, small-cell lung cancer, non-small cell lung cancer, renal cancer, ovarian cancer, renal cancer, or melanoma.

In certain embodiments, the cancer is a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, or hemangioblastoma.

In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, castrate resistant prostate cancer, castrate resistant metastatic prostate cancer, papillary thyroid carcinoma, follicular thyroid carcinoma, medullary thyroid carcinoma, or leiomyoma.

In certain embodiments, the cancer is bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In certain embodiments, the cancer is hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In certain embodiments, the cancer is hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is a solid tumor, such as a sarcoma, carcinoma, or lymphoma. In certain embodiments, the cancer is kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In certain embodiments, the cancer is renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In another aspect, this disclosure provides a method of causing death of a cancer cell. The method comprises contacting a cancer cell with an effective amount of a compound described herein, such as a compound of formula I or II, to cause death of the cancer cell.

In one aspect, the present disclosure provides a method of treating, preventing, and/or ameliorating cancer in a subject in need thereof. In certain embodiments, the cancer is charactered by a cancer cell having altered MGMT expression. In certain embodiments, the method comprises administering to the subject an agent that induces DNA lesions in the cell that lead to irreparable DNA damage to selectively treat the cancer.

The present disclosure further provides a method of treating, preventing, and/or ameliorating cancer in a subject, the method comprising administering to the subject a compound of the present disclosure or a pharmaceutical composition of the present disclosure.

In certain embodiments, the DNA lesion is a DNA double-strand break, a single-strand break, a stalled replication fork, a bulky adduct, or a lesion that further chemically reacts to form a irreparable DNA damage. In some embodiments, the irreparable DNA damage can be unrepaired lesions such as DNA inter- or intra-strand crosslinks.

In some embodiments, the agent does not affect MGMT proficient tissue. In other embodiments, the agent activity is independent of MMR protein expression and/or functional activity of the MMR pathway.

In certain embodiments, the cancer is selected from the group consisting of a glioma, colorectal cancer, non-small cell lung cancer, small cell lung cancer, sarcoma, pancreatic cancer, neuroendocrine tumor, esophageal cancer, lymphoma, head & neck cancer, breast cancer, bladder cancer, and leukemia. In other embodiments, the cancer is selected from the group consisting of an anaplastic astrocytoma, anaplastic oligodendroglioma, anaplastic ependymoma, medulloblastoma, and glioblastoma. In certain embodiments, the cancer is a glioma. In certain embodiments, the glioma is resistant to treatment with a DNA methylation agent and/or temozolomide. In certain embodiments, O⁶-methylguanine methyl transferase (MGMT)-silenced tumors are selectively killed.

In certain embodiments, the subject is resistant to treatment with an antineoplastic agent. Examples of antineoplastic agents include, but are not limited to temozolomide, procarbazine, altretamine, dacarbazine, mitozolomide, cisplatin, carboplatin, dicycloplatin, eptaplatin, lobaplatin, oxaliplatin, miriplatin, nedaplatin, triplatin tetranitrate, phenanthriplatin, Picoplatin, satraplatin, and lomustine.

In certain embodiments, the agent is an imidazotetrazine-based compound or a triazine-based compound. An imidazotetrazine-based compound is a compound having the following formula:

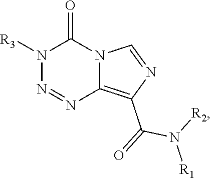

wherein each R₁, R₂, and R₃ group independently represent an optional substitution. In certain non-limiting embodiments, each R₁ and R₂ group is as defined in the compound of formula (I), formula (II), or formula (Ib). In certain embodiments, the R₃ group is CH₂CH₂F.

A triazine-based compound is a compound having the following formula:

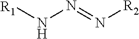

wherein each R¹ and R² each independently represent an optional substitution, as a non-limiting example, as provided in the compound of formula (Ib).

In certain embodiments, the agent that induces DNA lesions in the cell that lead to irreparable DNA damage to selectively treat the cancer is a compound of formula II, Ia, Ib, or Ic.

In accordance with the present disclosure, there is also provided herein a method for treating a patient having an MGMT deficient cancer. In certain embodiments, the method comprises administration to the patient of a therapeutically-effective amount of a compound of formula (II)

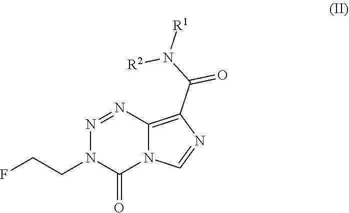

or a pharmaceutically acceptable salt thereof. In certain embodiments, R¹ is individually selected from H and lower alkyl. In certain embodiments, R² is individually selected from H, lower alkyl, trifluoroethyl,

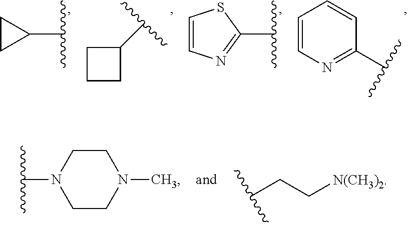

In certain embodiments, R¹ is H when R² is other than H or lower alkyl. In certain embodiments, R¹ and R² may combine to form —(CH₂)ₙ—. In certain embodiments, n is 3, 4, or 5. In certain embodiments, R¹ and R² together form —(CH₂)₂—N(CH₃)—(CH₂)₂—. The disclosed compounds are useful to treat cancers that are MGMT deficient regardless of MMR status. In various embodiments, a lower alkyl is a straight or branched C₁₋₄ alkyl group, or a C₁, C₂, C₃, or C₄ alkyl group.

In another embodiment provided herein, a compound of formula (I) is administered to a patient (e.g., a human patient) suffering from an MGMT deficient cancer. In another embodiment, the present method comprises administration of a therapeutically-effective amount of the compound of formula (I) to a patient suffering from an MGMT deficient, MMR deficient cancer, particularly a glioma. In another embodiment, the method comprises administering a therapeutically-effective amount of a compound of formula (I) to a patient suffering from an MGMT deficient cancer that is refractory (resistant) to treatment with TMZ. In some embodiments, the therapeutically effective amount of the compound is administered together with a pharmaceutically acceptable carrier. Suitable pharmaceutically acceptable carriers are well-known in the art, as discussed infra. A typical route of administration is oral, but other routes of administration are possible, as is well understood by those skilled in the medical arts. Administration may be by single or multiple doses. The amount of compound administered and the frequency of dosing may be optimized by the physician for the particular patient.

In addition to gliomas such as glioblastoma multiforme and brain lower grade glioma, the present method and compounds are useful to treat urothelial cancer, breast invasive carcinoma, colon adenocarcinoma, head and neck tumor (SCC), lung adenocarcinoma, rectum adenocarcinoma, and acute myeloid leukemia.

In some embodiments, the present methods and compounds are useful to treat glioma, colorectal cancer, non-small cell lung cancer, small cell lung cancer, sarcoma, pancreatic cancer, neuroendocrine tumor, esophageal cancer, lymphoma, head & neck cancer, breast cancer, bladder cancer, and leukemia. In preferred embodiments, the cancer is glioma.

In certain embodiments, the cancer treated by the present method is also either MMR deficient or unresponsive to treatment by temozolomide.

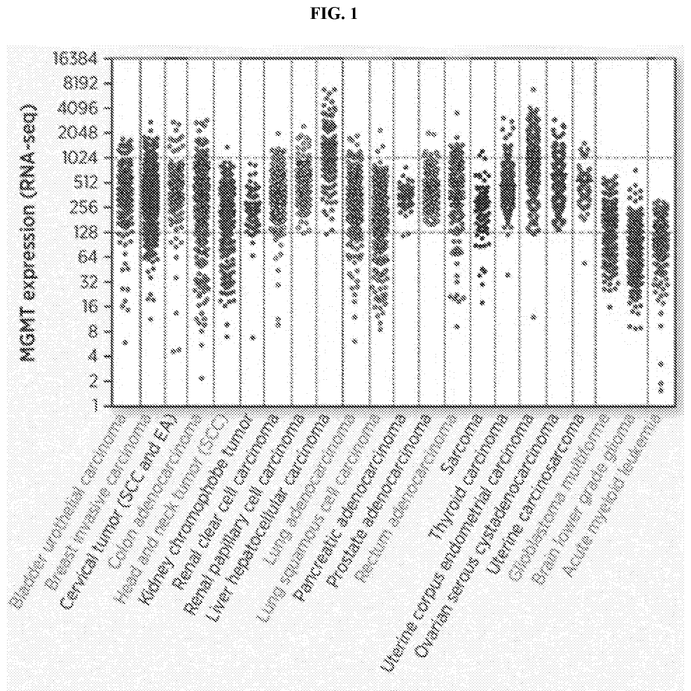

The present compounds and methods are useful for treatment, prevention, and/or amelioration of any cancer that is MGMT deficient, regardless of its MMR status, but are particularly applicable to treatment of cancers that are both MGMT and MMR deficient or that are both MGMT deficient and resistant to treatment by temozolomide. As shown in FIG. 1, many cancers have significant subpopulations that have critically reduced MGMT expression (i.e., that are MGMT deficient). Notable cancers among these are bladder urothelial cancer, breast invasive carcinoma, colon adenocarcinoma, head and neck tumor (SCC), lung adenocarcinoma, rectum adenocarcinoma, and acute myeloid leukemia. The present compounds and method are be particularly applicable to treatment of glioblastoma multiforme and brain lower grade glioma. As can be seen in FIG. 1, very significant subpopulations of these two cancers display critically reduced MGMT expression.

When treated with TMZ, cancers (particularly gliomas) often develop MMR deficiency and become resistant and unresponsive to further TMZ treatment. See, for example, Yu et al.—Temozolomide induced hypermutation is associated with distant recurrence and reduced survival after high-grade transformation of IDH-mutant low-grade gliomas—Neuro-Oncology 2021 Apr. 5; doi:10.1093/neuonc/noab081, and references cited therein. As described in Yu, a substantial number of gliomas treated with TMZ develop TMZ-induced hypermutation, and become resistant to further treatment with TMZ. The present compounds and methods provide an effective treatment for such cancers.

Without wishing to be bound thereby, in certain embodiments the disclosed compounds can act as bifunctional alkylation agents in a two-step process. The first reaction generates a primary DNA lesion (alkylation) that is rapidly removed by healthy MGMT-proficient cells. The second reaction slowly transforms the primary modification (alkylation) into a more toxic lesion via a unimolecular process. Thus, in certain embodiments the disclosed compounds can first alkylate 06-guanine and thereafter evolve slowly to more toxic inter-strand cross link (ICL), thereby establishing an MMR-independent method to amplify the therapeutic impact of MGMT deficiency.

Temozolomide has been shown to be very sensitive to even minor mutations in MMR proteins. McFaline-Figueroa, et al.—Minor changes in expression of the mismatch repair protein msh2 exert a major impact on glioblastoma response to temozolomide—Cancer Res 2015, 75, 312; and Nagel et al.—DNA Repair Capacity in Multiple Pathways Predicts Chemoresistance in Glioblastoma multiforme—Cancer Res 2017 Jan. 1; 77(1) 198-208. The present methods therefore allow treatment of patients having a cancer such as glioblastoma multiforme that is refractory to treatment with temozolomide, even if the cancer does not have more than one standard deviation lower abundance of the mRNA transcript for any of the MMR genes or the respective functional proteins and thus is not strictly “MMD deficient”.

Thus, the present disclosure provides methods for treating a patient having an MGMT deficient cancer that is refractory to treatment with temozolomide, comprising administration to the patient of a therapeutically-effective amount of a compound of formula (II)

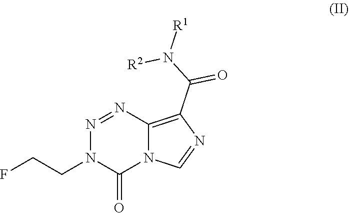

or a pharmaceutically acceptable salt thereof. In certain embodiments, R¹ is individually selected from H and lower alkyl. In certain embodiments, R² is individually selected from H, lower alkyl, trifluoroethyl,

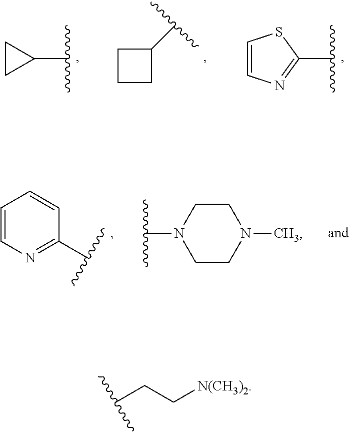

In certain embodiments, R¹ is H when R² is other than H or lower alkyl. In certain embodiments, R¹ and R² may combine to form —(CH₂)ₙ—. In certain embodiments, n is 3, 4, or 5. In certain embodiments, R¹ and R² form —(CH₂)₂—N(CH₃)—(CH₂)₂—. This method is particularly applicable to treatment of glioblastoma multiforme.

### IV. Combination Therapy

Another aspect of the invention provides for combination therapy. Compounds described herein (such as a compound of Formula I, or other compounds in Section II) or their pharmaceutically acceptable salts may be used in combination with additional therapeutic agents to treat medical disorders, such as cancer.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen more than 24 hours apart.

### Anti-Cancer Agents

Exemplary therapeutic agents that may be used as part of a combination therapy in treating cancer, include, for example, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

Radiation therapy may also be used as part of a combination therapy.

An additional class of agents that may be used as part of a combination therapy in treating cancer is immune checkpoint inhibitors (also referred to as immune checkpoint blockers). Immune checkpoint inhibitors are a class of therapeutic agents that have the effect of blocking immune checkpoints. See, for example, Pardoll in Nature Reviews Cancer (2012) vol. 12, pages 252-264. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAB3, (v) B7-H3, (vi) B7-H4, and (vii) TIM3. The CTLA4 inhibitor ipilumumab has been approved by the United States Food and Drug Administration for treating melanoma. In certain embodiments, the immune checkpoint inhibitor comprises pembrolizumab.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Accordingly, another aspect of the invention provides a method of treating cancer in a patient, where the method comprises administering to the patient in need thereof (i) a therapeutically effective amount of a compound described herein and (ii) a second anti-cancer agent, in order to treat the cancer, where the second therapeutic agent may be one of the additional therapeutic agents described above (e.g., mitomycin, tretinoin, ribomustin, gemcitabine, an immune checkpoint inhibitor, or a monoclonal antibody agent that targets non-checkpoint targets) or one of the following:
an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor;
an agonist of OX40, CD137, CD40, GITR, CD27, HVEM, TNFRSF25, or ICOS;
a therapeutic antibody targeting one of the following: CD20, CD30, CD33, CD52, EpCAM, CEA, gpA33, a mucin, TAG-72, CAIX, PSMA, a folate-binding protein, a ganglioside, Le, VEGF, VEGFR, VEGFR2, integrin αVβ3, integrin α5β1, EGFR, ERBB2, ERBB3, MET, IGF1R, EPHA3, TRAILR1, TRAILR2, RANKL, FAP, tenascin, CD19, KIR, NKG2A, CD47, CEACAM1, c-MET, VISTA, CD73, CD38, BAFF, interleukin-1 beta, B4GALNT1, interleukin-6, and interleukin-6 receptor;
a cytokine selected from IL-12, IL-15, GM-CSF, and G-CSF;
a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine; or
a placental growth factor, an antibody-drug conjugate, an oncolytic virus, or an anti-cancer vaccine.

In certain embodiments, the second anti-cancer agent is an ALK Inhibitor. In certain embodiments, the second anti-cancer agent is an ALK Inhibitor comprising ceritinib or crizotinib. In certain embodiments, the second anti-cancer agent is an ATR Inhibitor. In certain embodiments, the second anti-cancer agent is an ATR Inhibitor comprising AZD6738 or VX-970. In certain embodiments, the second anti-cancer agent is an A2A Antagonist. In certain embodiments, the second anti-cancer agent is a Base Excision Repair Inhibitor comprising methoxyamine. In certain embodiments, the second anti-cancer agent is a Base Excision Repair Inhibitor, such as methoxyamine. In certain embodiments, the second anti-cancer agent is a Bcr-Abl Tyrosine Kinase Inhibitor. In certain embodiments, the second anti-cancer agent is a Bcr-Abl Tyrosine Kinase Inhibitor comprising dasatinib or nilotinib. In certain embodiments, the second anti-cancer agent is a Bruton's Tyrosine Kinase Inhibitor. In certain embodiments, the second anti-cancer agent is a Bruton's Tyrosine Kinase Inhibitor comprising ibrutinib. In certain embodiments, the second anti-cancer agent is a CDC7 Inhibitor. In certain embodiments, the second anti-cancer agent is a CDC7 Inhibitor comprising RXDX-103 or AS-141.

In certain embodiments, the second anti-cancer agent is a CHK1 Inhibitor. In certain embodiments, the second anti-cancer agent is a CHK1 Inhibitor comprising MK-8776, ARRY-575, or SAR-020106. In certain embodiments, the second anti-cancer agent is a Cyclin-Dependent Kinase Inhibitor. In certain embodiments, the second anti-cancer agent is a Cyclin-Dependent Kinase Inhibitor comprising palbociclib. In certain embodiments, the second anti-cancer agent is a DNA-PK Inhibitor. In certain embodiments, the second anti-cancer agent is a DNA-PK Inhibitor comprising MSC2490484A. In certain embodiments, the second anti-cancer agent is Inhibitor of both DNA-PK and mTOR. In certain embodiments, the second anti-cancer agent comprises CC-115.

In certain embodiments, the second anti-cancer agent is a DNMT1 Inhibitor. In certain embodiments, the second anti-cancer agent is a DNMT1 Inhibitor comprising decitabine, RX-3117, guadecitabine, NUC-8000, or azacytidine. In certain embodiments, the second anti-cancer agent comprises a DNMT1 Inhibitor and 2-chloro-deoxyadenosine. In certain embodiments, the second anti-cancer agent comprises ASTX-727.

In certain embodiments, the second anti-cancer agent is a HDAC Inhibitor. In certain embodiments, the second anti-cancer agent is a HDAC Inhibitor comprising OBP-801, CHR-3996, etinostate, resminostate, pracinostat, CG-200745, panobinostat, romidepsin, mocetinostat, belinostat, AR-42, ricolinostat, KA-3000, or ACY-241.

In certain embodiments, the second anti-cancer agent is a Hedgehog Signaling Pathway Inhibitor. In certain embodiments, the second anti-cancer agent is a Hedgehog Signaling Pathway Inhibitor comprising sonidegib or vismodegib. In certain embodiments, the second anti-cancer agent is an IDO Inhibitor. In certain embodiments, the second anti-cancer agent is an IDO Inhibitor comprising INCB024360. In certain embodiments, the second anti-cancer agent is a JAK Inhibitor. In certain embodiments, the second anti-cancer agent is a JAK Inhibitor comprising ruxolitinib or tofacitinib. In certain embodiments, the second anti-cancer agent is a mTOR Inhibitor. In certain embodiments, the second anti-cancer agent is a mTOR Inhibitor comprising everolimus or temsirolimus. In certain embodiments, the second anti-cancer agent is a MEK Inhibitor. In certain embodiments, the second anti-cancer agent is a MEK Inhibitor comprising cobimetinib or trametinib. In certain embodiments, the second anti-cancer agent is a MELK Inhibitor. In certain embodiments, the second anti-cancer agent is a MELK Inhibitor comprising ARN-7016, APTO-500, or OTS-167. In certain embodiments, the second anti-cancer agent is a MTH1 Inhibitor. In certain embodiments, the second anti-cancer agent is a MTH1 Inhibitor comprising (S)-crizotinib, TH287, or TH588.

In certain embodiments, the second anti-cancer agent is a PARP Inhibitor. In certain embodiments, the second anti-cancer agent is a PARP Inhibitor comprising MP-124, olaparib, BGB-290, talazoparib, veliparib, niraparib, E7449, rucaparb, or ABT-767. In certain embodiments, the second anti-cancer agent is a Phosphoinositide 3-Kinase Inhibitor. In certain embodiments, the second anti-cancer agent is a Phosphoinositide 3-Kinase Inhibitor comprising idelalisib. In certain embodiments, the second anti-cancer agent is an inhibitor of both PARP1 and DHODH (i.e., an agent that inhibits both poly ADP ribose polymerase 1 and dihydroorotate dehydrogenase).

In certain embodiments, the second anti-cancer agent is a Proteasome Inhibitor. In certain embodiments, the second anti-cancer agent is a Proteasome Inhibitor comprising bortezomib or carfilzomib. In certain embodiments, the second anti-cancer agent is a Topoisomerase-II Inhibitor. In certain embodiments, the second anti-cancer agent is a Topoisomerase-II Inhibitor comprising vosaroxin.

In certain embodiments, the second anti-cancer agent is a Tyrosine Kinase Inhibitor. In certain embodiments, the second anti-cancer agent is a Tyrosine Kinase Inhibitor comprising bosutinib, cabozantinib, imatinib or ponatinib. In certain embodiments, the second anti-cancer agent is a VEGFR Inhibitor. In certain embodiments, the second anti-cancer agent is a VEGFR Inhibitor comprising regorafenib. In certain embodiments, the second anti-cancer agent is a WEE1 Inhibitor. In certain embodiments, the second anti-cancer agent is a WEE1 Inhibitor comprising AZD1775.

In certain embodiments, the second anti-cancer agent is an agonist of OX40, CD137, CD40, GITR, CD27, HVEM, TNFRSF25, or ICOS. In certain embodiments, the second anti-cancer agent is a therapeutic antibody selected from the group consisting of rituximab, ibritumomab tiuxetan, tositumomab, obinutuzumab, ofatumumab, brentuximab vedotin, gemtuzumab ozogamicin, alemtuzumab, IGN101, adecatumumab, labetuzumab, huA33, pemtumomab, oregovomab, minetumomab, cG250, J591, Mov18, farletuzumab, 3F8, ch14.18, KW-2871, hu3S193, lgN311, bevacizumab, IM-2C6, pazopanib, sorafenib, axitinib, CDP791, lenvatinib, ramucirumab, etaracizumab, volociximab, cetuximab, panitumumab, nimotuzumab, 806, afatinib, erlotinib, gefitinib, osimertinib, vandetanib, trastuzumab, pertuzumab, MM-121, AMG 102, METMAB, SCH 900105, AVE1642, IMC-A12, MK-0646, R1507, CP 751871, KB004, IIIA-4, mapatumumab, HGS-ETR2, CS-1008, denosumab, sibrotuzumab, F19, 81C6, MEDI551, lirilumab, MEDI9447, daratumumab, belimumab, canakinumab, dinutuximab, siltuximab, and tocilizumab.

In certain embodiments, the second anti-cancer agent is a placental growth factor. In certain embodiments, the second anti-cancer agent is a placental growth factor comprising ziv-aflibercept. In certain embodiments, the second anti-cancer agent is an antibody-drug conjugate. In certain embodiments, the second anti-cancer agent is an antibody-drug conjugate selected from the group consisting of brentoxumab vedotin and trastuzumab emtransine.

In certain embodiments, the second anti-cancer agent is an oncolytic virus. In certain embodiments, the second anti-cancer agent is the oncolytic virus talimogene laherparepvec. In certain embodiments, the second anti-cancer agent is an anti-cancer vaccine. In certain embodiments, the second anti-cancer agent is an anti-cancer vaccine selected from the group consisting of a GM-CSF tumor vaccine, a STING/GM-CSF tumor vaccine, and NY-ESO-1. In certain embodiments, the second anti-cancer agent is a cytokine selected from IL-12, IL-15, GM-CSF, and G-CSF.

In certain embodiments, the second anti-cancer agent is a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine.

### Additional Considerations

The doses and dosage regimen of the active ingredients used in the combination therapy may be determined by an attending clinician. In certain embodiments, the compound described herein (such as a compound of Formula I, or other compounds in Section II) and the additional therapeutic agent(s) are administered in doses commonly employed when such agents are used as monotherapy for treating the disorder. In other embodiments, the compound described herein (such as a compound of Formula I, or other compounds in Section II) and the additional therapeutic agent(s) are administered in doses lower than the doses commonly employed when such agents are used as monotherapy for treating the disorder. In certain embodiments, the compound described herein (such as a compound of Formula I, or other compounds in Section II) and the additional therapeutic agent(s) are present in the same composition, which is suitable for oral administration.

In certain embodiments, the compound described herein (such as a compound of Formula I, or other compounds in Section II) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of the compound described herein (such as a compound of Formula I, or other compounds in Section II), a pharmaceutically acceptable carrier, vehicle or diluent, and optionally at least one additional therapeutic agent listed above.

### V. Pharmaceutical Formulations

In one aspect, the present disclosure provides a pharmaceutical composition comprising a compound described herein (e.g., a compound of formula (I) or formula (II)) and a pharmaceutically acceptable carrier.

In certain embodiments, pharmaceutical compositions suitable for use for the compounds and in the methods described herein can include a disclosed compound and a pharmaceutically acceptable carrier or diluent.

The composition may be formulated for intravenous, subcutaneous, intraperitoneal, intramuscular, topical, oral, buckle, nasal, pulmonary or inhalation, ocular, vaginal, or rectal administration. In some embodiments, the compounds are formulated for oral administration. The pharmaceutical composition can be formulated to be an immediate-release composition, sustained-release composition, delayed-release composition, etc., using techniques known in the art.

Pharmacologically acceptable carriers for various dosage forms are known in the art. For example, excipients, lubricants, binders, and disintegrants for solid preparations are known; solvents, solubilizing agents, suspending agents, isotonicity agents, buffers, and soothing agents for liquid preparations are known. In some embodiments, the pharmaceutical compositions include one or more additional components, such as one or more preservatives, antioxidants, stabilizing agents and the like.

Additionally, the disclosed pharmaceutical compositions can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In some embodiment, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Pharmaceutical compositions of the disclosure can be administered in combination with other therapeutics that are part of the current standard of care for cancer.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of cancer in a patient.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

### Medical Uses and Preparation of a Medicament

In another aspect, the disclosure provides for the use of a compound described herein (such as a compound of formula I or other compounds in Section II) in the manufacture of a medicament. In certain embodiments, the medicament is for treating a disease or condition described herein.

In another aspect, the disclosure provides for the use of a compound described herein (such as a compound of formula I or other compounds in Section II) for treating a disease or condition, such as a disease or condition described herein.

### VI. Administration

Routes of administration of any of the compositions described herein include oral, nasal, rectal, intravaginal, parenteral (e.g., IM, IV and SC), buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

The regimen of administration may affect what constitutes an effective amount. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to treat the disorder involving overexpression of methylene tetrahydrofolate dehydrogenase 2 (MTHFD2) in a subject. An effective amount of the therapeutic compound necessary to achieve a therapeutic effect may vary according to factors such as the state of the disease or disorder in the subject; the age, sex, and weight of the subject; and the ability of the therapeutic compound to treat the disease or disorder in the subject. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound useful within the invention is from about 1 and 5,000 mg/kg of body weight/per day. One of ordinary skill in the art would be able to study the relevant factors and make the determination regarding the effective amount of the therapeutic compound without undue experimentation.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY—P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 6%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.010% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as such as lauryl, stearyl, or oleyl alcohols, or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The present invention also includes a multilayer tablet comprising a layer providing for the delayed release of one or more compounds useful within the invention, and a further layer providing for the immediate release of a medication for a disorder involving overexpression of methylene tetrahydrofolate dehydrogenase 2 (MTHFD2) in a subject. Using a wax/pH-sensitive polymer mix, a gastric insoluble composition may be obtained in which the active ingredient is entrapped, ensuring its delayed release.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In a preferred embodiment of the invention, the compounds useful within the invention are administered to a subject, alone or in combination with another pharmaceutical agent, using a sustained release formulation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Therapeutically Effective Doses and Dosing Regimens

The therapeutically effective amount or dose of a compound of the present invention will depend on the age, sex and weight of the subject, the current medical condition of the subject and the nature of the disorder involving overexpression of methylene tetrahydrofolate dehydrogenase 2 (MTHFD2) being treated. The skilled artisan will be able to determine appropriate dosages depending on these and other factors.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

In particular, the selected dosage level depends upon a variety of factors, including the activity of the particular compound employed, the time of administration, the rate of excretion of the compound, the duration of the treatment, other drugs, compounds or materials used in combination with the compound, the age, sex, weight, condition, general health and prior medical history of the subject being treated, and like factors well, known in the medical arts.

A medical doctor, e.g., physician or veterinarian, having ordinary skill in the art may readily determine and prescribe the effective amount of the pharmaceutical composition required. For example, the physician or veterinarian may start doses of the compounds useful within the invention employed in the pharmaceutical composition at levels lower than that required in order to achieve the desired therapeutic effect and gradually increase the dosage until the desired effect is achieved.

In certain embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on the unique characteristics of the therapeutic compound and the particular therapeutic effect to be achieved, and the limitations inherent in the art of compounding/formulating such a therapeutic compound for the treatment of a disorder involving overexpression of methylene tetrahydrofolate dehydrogenase 2 (MTHFD2) in a subject.

In some embodiments, the therapeutically effective dose of the compound may be administered every day, for 21 days followed by a 7 day rest, every 7 days with a 7 day rest in between each dosage period, or for 5 continuous days followed by a 21 day rest, in each instance referring to a 28 day dosage cycle.

The therapeutically effective dose of compound administered to the patient (whether administered in a single does or multiple doses) should be sufficient to treat the cancer. Such therapeutically effective amount may be determined by evaluating the symptomatic changes in the patient.

Exemplary doses can vary according to the size and health of the individual being treated, the condition being treated, and the dosage regimen adopted. In some embodiments, the effective amount of a disclosed compound per 28 day dosage cycle is about 1.5 g/m²; however, in some situations the dose may be higher or lower—for example 2.0 g/m² or 1.0 g/m². The daily dose may vary depending on (inter alia) the dosage regimen adopted. For example, if the regimen is dosing for five days followed by a 21 day rest and the total dosage per 28 day cycle is 1.0 g/m², then the daily dose would be 200 mg/m². Alternatively, if the regimen is dosing for 21 days followed by a 5 day rest and the total dosage per 28 day cycle is 1.6 g/m², then the daily dose would be 75 mg/m². Similar results would obtain for other dosage regimens and total 28 day doses.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days.

The compounds for use in the method of the invention may be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

The disclosed methods of treatment may also be combined with other known methods of treatment as the situation may require.

Therapeutically effective doses and dosing regimens of the foregoing methods may vary, as would be readily understood by those of skill in the art. Dosage regimens may be adjusted to provide the optimum desired response. For example, in some embodiments, a single bolus dose of the compound may be administered, while in some embodiments, several divided doses may be administered over time, or the dose may be proportionally reduced or increased in subsequent dosing as indicated by the situation.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 and so forth, as well as individual numbers within that range, for example, 1, 2, 2.7, 3, 4, 5, 5.1, 5.3, 5.5, and 6. This applies regardless of the breadth of the range.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

General Chemical Experimental Procedures. All reactions were performed in single-neck, flame dried round-bottom flasks fitted with rubber septa under a positive pressure of argon, unless otherwise specified. Air- and moisture-sensitive liquids were transferred via syringe or stainless-steel cannula. Organic solutions were concentrated by rotary evaporation at 31° C., unless otherwise noted. Flash-column chromatography was performed as described by Still et al., employing silica gel (SiliaFlash® P60, 60 Å, 40-63 μm particle size) purchased from Silicycle (Quebec, Canada). Analytical thin-layered chromatography (TLC) was performed using glass plates pre-coated with silica gel (250 μm, 60 Å pore size) embedded with a fluorescent indicator (254 nm). TLC plates were visualized by exposure to ultraviolet (UV) light.

Chemical Materials. Commercial solvents, chemicals, and reagents were used as received with the follow exceptions. Dichloromethane, tetrahydrofuran, and toluene were purified according to the method of Pangbom et al. Triethylamine was distilled from calcium hydride under an atmosphere of nitrogen immediately prior to use. N,N-Di-iso-propylethylamine was distilled from calcium hydride under argon immediately prior to use. The diazonium S7, the imidazolyl triazene 1b, the imidazolyl triazene 4b, the imidazolyl triazene 9, the imidazolyl triazene 12b, and the imidazolyl triazene 13 were synthesized according to published procedures.

Chemistry Instrumentation. Proton nuclear magnetic resonance (¹H NMR) were recorded at 400 or 600 megahertz (MHz) at 23° C., unless otherwise noted. Chemical shifts are expressed in parts per million (ppm, 6 scale) downfield from tetramethylsilane and are referenced to residual proton in the NMR solvent ((CD₃)SO(CHD₂), δ 2.50). Data are represented as follows: chemical shift, multiplicity (s=singlet, d=doublet, t=triplet, q=quartet, m=multiplet and/or multiple resonances, b=broad, app=apparent), coupling constant in Hertz (Hz), integration, and assignment. Proton-decoupled carbon nuclear magnetic resonance spectra (¹³C NMR) were recorded at 150 MHz at 23° C., unless otherwise noted. Chemical shifts are expressed in parts per million (ppm, (scale) downfield from tetramethylsilane and are referenced to the carbon resonances of the solvent (DMSO-d₆, δ 39.52). ¹H-¹H gradient-selected correlation spectroscopy (COSY), ¹H-¹³C heteronuclear single quantum coherence (HSQC), and ¹H-¹³C gradient-selected heteronuclear multiple bond correlation (gHMBC) were recorded at 600 MHz at 23° C., unless otherwise noted. Carbon-decoupled fluorine nuclear magnetic resonance spectra (¹⁹F NMR) were recorded at 396 MHz at 23° C., unless otherwise noted. Chemical shifts are expressed in parts per million (ppm, 6 scale) downfield from tetramethylsilane. Attenuated total reflectance Fourier transform infrared (ATR-FTIR) spectra were obtained using a Thermo Electron Corporation Nicolet 6700 FTIR spectrometer referenced to a polystyrene standard. Data are represented as follows: frequency of absorption (cm⁻¹), intensity of absorption (s=strong, m=medium, w=weak, br=broad). Analytical liquid chromatography-mass spectroscopy (LCMS) was performed on a Waters instrument equipped with a reverse-phase Cis column (1.7 μm particle size, 2.1×50 mm). Samples were eluted with a linear gradient of 5% acetonitrile-water containing 0.1% formic acid→100% acetonitrile containing 0.1% formic acid over 0.75 min, followed by 100% acetonitrile containing 0.1% formic acid for 0.75 min, at a flow rate of 800 μL/min. HRMS were obtained on a Waters UPLC/HRMS instrument equipped with a dual API/ESI high resolution mass spectrometry detector and photodiode array detector.

Biological Materials. Temozolomide (TMZ, 1a), lomustine (14), O⁶-benzylguanine (O⁶BG), doxorubicin, and olaparib were purchased from Selleck Chemicals. Methylmethane sulfonate (MMS) was purchased from Alfa-Aesir. Mitozolomide (MTZ, 12a) was purchased from Enamine. Mitomycin C (MMC), N-ethylmaleimide (NEM), N-acetyl-L-cysteine (NAC), and cisplatin were purchased from Sigma. TMZ (1a, 100 mM stock), O⁶BG (100 mM stock), MTZ (12a, 100 mM stock), MMS (500 mM stock) and NAC (100 mM stock) were dissolved in DMSO and stored at −80° C. MMC (10 mM stock), lomustine (14, 100 mM stock), doxorubicin (10 mM stock), and olaparib (18.3 mM stock) were dissolved in DMSO and stored at −20° C. NEM (400 mM stock) was dissolved in EtOH and stored at −20° C. Cisplatin (5 mM stock) was dissolved in H₂O and stored at 4° C. for up to 7 days.

Cell Culture. LN229 MGMT− and MGMT+ cell lines were a gift from B. Kaina (Johannes Gutenberg University Mainz, Mainz, Germany) and grown in DMEM with 10% FBS (Gibco). DLD1 BRCA2+/− and BRCA2−/− cell lines (Horizon Discovery, Cambridge, UK) were grown in RPMI 1640 with 10% FBS. HCT116 MLH1−/− and HCT116+Chr3 cell lines were a gift from T. Kunkel (National Institute of Environmental Health Sciences, Durham, NC) and grown in DMEM with 10% FBS, with 0.5 μg/mL G418 (Sigma) for HCT116+Chr3 cells. PD20 cell lines complemented with empty vector (+EV), wildtype FANCD2 (+FD2), or K561R ubiquitination-mutant FANCD2 (+KR) were a gift from G. Kupfer and P. Glazer (Yale University, New Haven, CT) and growth in DMEM with 10% FBS. PEO1 and PEO4 cell lines were a gift from T. Taniguchi (Fred Hutchinson Cancer Research Center, Seattle, WA) and were grown in DMEM with 10% FBS. BJ fibroblasts (normal human fibroblast cells) were purchased from ATCC (CRL-2522) and grown in DMEM with 10% FBS. NER isogenic MEFs were a gift from F. Rogers (Yale University, New Haven, CT) and were grown in DMEM with 10% FBS. All human cell lines were validated by short tandem repeat profiling (excluding BJ fibroblasts which were used within 6 passages of receiving from ATCC) and confirmed negative for mycoplasma by quantitative RT-PCR.

MMR Protein shRNA Knockdown. pGIPZ lentiviral shRNA vectors targeting MSH2, MSH6, MLH1, PMS2, and MSH3 were purchased from Horizon Discovery (Table S2). Lentiviral particles were produced in HEK293T cells via co-transfection with lentiviral shRNA plasmid, pCMV-VSV-G envelope plasmid (Addgene, #8454) and psPAX2 packaging plasmid (Addgene, #12260), using Lipofectamine 3000 Reagent (Invitrogen, L3000001) per manufacturer's protocol. Viral particles were harvested 48 h post-transfection and used to transduce LN229 MGMT+/− cells in the presence of 8 μg/mL polybrene. Selection of pooled cells with lentiviral expression was established with 1 μg/mL puromycin 48 h post-transduction for 3 to 4 days. Single cell cloning was performed by limiting dilution and protein knockdown was confirmed by western blotting.

TABLE 1
pGIPZ Lentivral shRNA Vectors for MMR protein
knockdown

| | pGIPZ Human | |
|---|---|---|
| Protein | Lentiviral | Mature Antisense |
| Target | shRNA Clone | Sequence |
| MSH2 | RHS4430-200305416 | TTACTAAGCACAACACTCT |
| MSH6 | RHS4430-200281418 | TACACATTACTTTGAATCC |
| MLH1 | RHS4430-200268977 | AACTGAGAAACTAATGCCT |
| PMS2 | RHS4430-200253216 | TTCACAGCTACATCAACCT |
| MSH3 | RHS4430-200158125 | TTTCTTGCAAATGCATTCG |

Western Blotting. For phospho-protein analysis experiments, cells were lysed in 1× RIPA buffer (Cell Signaling Technology, #9806) supplemented with 1× Protease Inhibitor Cocktail (Roche) and 1× PhosSTOP Phosphatase Inhibitor Cocktail (Sigma). For all other western blot analyses, cells were lysed in lysis buffer (50 mM HEPES, 250 mM NaCl, 5 mM EDTA, 1% NP-40) supplemented with 1× Protease Inhibitor Cocktail (Roche). The de-ubiquitination inhibitor N-ethylmaleimide (NEM, 4 mM) was added in FANCD2 ubiquitination analysis experiments. Proteins were separated using NuPAGE 4-12% Bis-Tris or 3-8% Tris-Acetate Gels (Invitrogen) and transferred to Immobilon-P PVDF membrane (Millipore) for western blotting. Membranes were blocked with 5% milk in TBS-T for 1 h prior to primary antibody addition overnight at 4° C. Primary antibodies were used under the following conditions: mouse anti-CHK1 (Cell Signaling Technology, #2360), 1/1000 in 5% milk; rabbit anti-CHK2 (Cell Signaling Technology, #6334), 1/1000 in 5% BSA; rabbit anti-FANCD2 (Cell Signaling Technology, #16323), 1/1000 in 5% BSA; HRP-conjugated anti-GAPDH (ProteinTech HRP-60004), 1/10,000 in 5% milk; rabbit anti-MGMT (Cell Signaling Technology, #2739), 1/1000 in 5% BSA; rabbit anti-MLH1 (Cell Signaling Technology, #4256), 1/1000 in 5% BSA; mouse anti-MSH2 (Cell Signaling Technology, #2850), 1/1000 in 5% milk; mouse anti-MSH3 (BD Biosciences, BD611390), 1/500 in 5% milk; mouse anti-MSH6 (BD Biosciences, BD610918), 1/1000 in 5% milk; rabbit anti-phospho-CHK1 (S345) (Cell Signaling Technology, #2341), 1/1000 in 5% BSA; rabbit anti-phospho-CHK2 (T68) (Cell Signaling Technology, #2661), 1/1000 in % BSA; mouse anti-PMS2 (Santa Cruz, sc-25315), 1/100 in 5% milk; mouse anti-Vinculin (Santa Cruz, sc-25336), 1/1000 in 5% milk. Anti-mouse IgG HRP-conjugated antibody (Cell Signaling Technology, #7076) and anti-rabbit IgG HRP-conjugated antibody (Cell Signaling Technology, #93702) were added at 1/5000 in 5% milk for 1 h. Chemiluminescence detection was performed with Clarity Max Western ECL Substrate (Bio-Rad) and blots were imaged on a ChemiDoc XRS+ Molecular Imager (Bio-Rad). Where shown, bands were quantified using ImageJ software.

Short-term Cell Viability Assay. Cells were seeded in 96-well plates at 1000 or 2000 cells/well and allowed to adhere at 23° C. for 60 min and then incubated overnight at 37° C. Cells were treated with indicated concentrations of compounds in triplicate for 4-6 days prior to fixation with 3.7% paraformaldehyde and nuclear staining with 1 μg/mL Hoechst 33342 dye. Cells were imaged on a Cytation 3 imaging reader (BioTek) and quantified using CellProfiler software.

Clonogenic Cell Survival Assay. Cells were trypsinized, washed, counted, and diluted in a medium containing various concentrations of drug. They were then immediately seeded in six-well plates in triplicate at three-fold dilutions, ranging from 9000 to 37 cells per well. Depending on colony size, these plates were kept in the incubator for 10 to 14 days. After incubation, colonies were washed in phosphate-buffered saline (PBS), stained with crystal violet, counted, and quantified.

IR Alkaline Comet Assay. Assay was performed utilizing the CometAssay Kit (Trevigen) according to the alkaline assay protocol, with the addition of slide irradiation post-lysis. Cells were trypsinized, washed with 1×PBS, added to melted Comet LMAgarose (Trevigen), and spread on Trevigen CometSlides at a density of 1000 cells per sample in 50 μL. Lysis solution (Trevigen) with 10% DMSO was added overnight at 4° C. Slides were removed from lysis buffer and irradiated to 0 or 10 Gy using an XRAD 320 X-Ray System (Precision X-Ray) at 320 kV, 12.5 mA, and 50.0 cm SSD, with a 2 mm Al filter and 20 cm×20 cm collimator. Slides were then placed in alkaline buffer (200 mM NaOH, 1 mM EDTA) for 45 min, followed by electrophoresis in 850 mL alkaline buffer for 45 min at 4° C. Slides were washed and stained with SYBR gold (Invitrogen) per Trevigen assay protocol. Slides were imaged on a Cytation 3 imaging reader (BioTek), and comets were analyzed using CometScore 2.0 software (TriTek).

Genomic DNA Denaturing Gel Electrophoresis. Cells were trypsinized, washed with 1×PBS, and stored at −80° C. prior to processing. Genomic DNA was extracted with the DNeasy Blood & Tissue Kit (Qiagen) per kit protocol. A 0.7% agarose gel was prepared in 100 mM NaCl-2 mM EDTA (pH 8) and soaked in 40 mM NaOH-1 mM EDTA running buffer for 2 h. Genomic DNA (400 ng/well) was then loaded in 1× BlueJuice loading buffer (Invitrogen) and subjected to electrophoresis at 2 V/cm for 30 min, followed by 3 V/cm for 2 h. The gel was neutralized in 150 mM NaCl-100 mM Tris (pH 7.4) for 30 min, twice, and then stained with 1× SYBR Gold in 150 mM NaCl-100 mM Tris (pH 7.4) for 90 min. Imaging was performed on a ChemiDoc XRS+ Molecular Imager (Bio-Rad).

Plasmid Linearization Assay. To set up the linearization reactions, 20 units of EcoRI-HF (New England Biolabs) was mixed with 20 μg 2686 bp pUC19 vector DNA in CutSmart buffer (New England Biolabs), pH 7.9, in a total volume of 1000 μL for 30 min at 37° C. The CutSmart buffer contains 50 mM potassium acetate, 20 mM Tris acetate, 10 mM magnesium acetate, and 100 μg/mL BSA. The reacted DNA was then purified using PCR cleanup kit and quantified using the NanoDrop One (Thermo Fisher). The DNA was then stored at −20° C. before use in in vitro DNA cross-linking assays or melting temperature analysis.

In Vitro DNA Cross-linking Assays. Linearized pUC19 DNA, prepared as described above, was used for in vitro DNA cross-linking assays. For each condition, 200 ng of linearized pUC19 DNA (15.4 μM base pairs) was incubated with the indicated concentration of drug in 20 μL. Drug stock concentrations were made in DMSO such that each reaction contained a fixed 5% DMSO concentration. Reactions were conducted in 100 mM Tris buffer (pH 7.4). Cisplatin (Sigma) and DMSO vehicle were used as positive and negative controls, respectively. Reactions were conducted between 3-96 h at 37° C. The DNA was stored at −80° C. until electrophoretic analysis. For gel electrophoresis, DNA concentration was preadjusted to 10 ng/μL. Five microliters (50 ng) of the DNA solution was removed and mixed with 1.5 μL of 6× purple gel loading dye, no SDS, and loaded onto 1% agarose Tris Borate EDTA TBE gels. For denaturing gels, 5 μL (50 ng) of the DNA solution was removed and mixed with 15 μL of 0.2% denaturing buffer (0.27% sodium hydroxide, 10% glycerol, and 0.013% bromophenol blue) or 0.4% denaturing buffer (0.53% sodium hydroxide, 10% glycerol, and 0.013% bromophenol blue) in an ice bath. The mixed DNA samples were denatured at 4° C. for 5 min and then immediately loaded onto a 1% agarose Tris Borate EDTA (TBE) gel. All gel electrophoresis was conducted at 90 V for 2 h (unless otherwise noted). The gel was stained with SYBR Gold (Invitrogen) for 2 h.

EndoIV Depurination Assay. For each condition, 200 ng of supercoiled pUC19 DNA (15.4 μM base pairs) was incubated with the indicated concentration of drug in 20 μL for 3 hours. Drug stock concentrations were made in DMSO such that each reaction contained a fixed 5% DMSO concentration. Reactions were conducted in 100 mM Tris buffer (pH 7.4). For each EndoIV reaction, 50 ng of processed DNA was mixed with 20 units of EndoIV in NEBuffer 3.1 (New England Biolabs), pH 7.9, in a total volume of 20 μL for 16-20 h (unless otherwise noted) at 37° C. The NEBuffer 3.1 contained 100 mM sodium chloride, 50 mM Tris-HCl, 10 mM magnesium chloride, and 100 μg/mL BSA. For each negative control, 50 ng of processed DNA was mixed with NEBuffer 3.1, pH 7.9, in a total volume of 20 μL for 16-20 h (unless otherwise noted) at 37° C. Following completion of the experiment, the DNA was stored at −20° C. before electrophoretic analysis.

Melting Temperature Assay. Linearized pUC19 DNA (750 ng), prepared as described above, was incubated with the indicated concentration of either MMS or KL-50 (4a) adjusted in a final volume of 18 μL in 100 mM Tris buffer (pH 7.4) for 3 h. Drug stock concentrations were made in DMSO such that each reaction contained a fixed 5% DMSO concentration. Afterwards, 1 μL each of 20× SYBR Green dye (Invitrogen) and 20× ROX reference dye (Invitrogen) was added and melting temperature analysis was run on a StepOnePlus RT PCR System (Applied Biosciences) to generate melting temperature curves.

Immunofluorescence Foci Assays. High-throughput immunofluorescence foci assays were performed at the Yale Center for Molecular Discovery (YCMD). Cells were seeded at 2000 cells/well in black polystyrene flat bottom 384-well plates (Greiner Bio-One) and allowed to adhere overnight. Compound addition was performed utilizing a Labcyte Echo 550 liquid handler (Beckman Coulter), with 6 replicates per test condition and 12 replicates per control condition. Following drug incubation, cells were fixed and stained for phospho-SER139-H2AX (γH2AX), 53BP1, or phospho-SER33-RPA2 (pRPA) as follows.

γH2AX protocol: Cells were fixed with 4% paraformaldehyde in 1×PBS for 15 min, washed twice with 1×PBS, incubated in extraction buffer (0.5% Triton X-100 in 1×PBS) for 10 min, washed twice with 1×PBS, and incubated in blocking buffer (Blocker Casein in PBS, Thermo Scientific+5% goat serum, Life Technologies) for 1 h. Mouse anti-phospho-histone H2A.X (Ser139) antibody (clone JBW301, Millipore, 05-636) was added 1/1000 in blocking buffer at 4° C. overnight. After washing with 1×PBS, cells were incubated with goat anti-mouse IgG (H+L) highly cross-adsorbed secondary antibody, Alexa Fluor 647 (Invitrogen, A-21236) 1/500 and with 1 μg/mL Hoechst nucleic acid dye in blocking buffer for 2 h, and then washed with 1×PBS.

53BP1 protocol: Cells were fixed with 4% paraformaldehyde+0.02% Triton X-100 in 1×PBS for 20 minutes, washed twice with 1×PBS, and incubated in blocking buffer (10% FBS, 0.5% Triton X-100 in 1×PBS) for 1 h. Rabbit anti-53BP1 antibody (Novus Biologicals, NB100-904) was added 1/1000 in blocking buffer at 4° C. overnight. After washing with 1×PBS, cells were incubated with goat anti-rabbit IgG (H+L) highly cross-adsorbed secondary antibody, Alexa Fluor 647 (Invitrogen, A-21245) 1/500 and with 1 μg/mL Hoechst nucleic acid dye in blocking buffer for 2 h, and then washed with 1×PBS.

pRPA protocol: Cells were washed twice with 1×PBS on ice, incubated in extraction buffer (0.5% Triton X-100 in 1×PBS) for 5 min on ice, fixed with 3% paraformaldehyde+2% sucrose in 1×PBS for 15 min at 23° C., incubated again in extraction buffer for 5 min on ice, and incubated in blocking buffer (2% BSA, 10% milk, 0.1% Triton X-100 in 1×PBS) for 1 h at 23° C. Rabbit anti-phospho-RPA2 (S33) antibody (Bethyl Laboratories, A300-246A) was added 1/1000 in blocking buffer at 4° C. overnight. After washing 4 times with IF wash buffer (0.1% Triton X-100 in 1×PBS), cells were incubated with goat anti-rabbit IgG (H+L) highly cross-adsorbed secondary antibody, Alexa Fluor 647 (Invitrogen, A-21245) 1/500 and with 1 μg/mL Hoechst nucleic acid dye in blocking buffer for 1 h at 37° C. Cells were washed twice with IF wash buffer and twice with 1×PBS.

Imaging was performed on an InCell Analyzer 2200 Imaging System (GE Corporation) at 40× magnification. Twenty fields-of-view were captured per well. Foci analysis was performed using InCell Analyzer software (GE Corporation). Outer wells were excluded from analysis to limit variation due to edge effects.

Additional small scale immunofluorescence assays used for extended time course analysis of γH2AX foci were performed in Millicell EZSLIDE 8-well chamber slides (Millipore). Cells were seeded at 10,000 cells/well and allowed to adhere overnight. Following drug treatment, cells were fixed and stained for γH2AX as described above, without the addition of Hoechst dye. Slides were mounted with Vectashield Antifade Mounting Medium with DAPI (Vector Laboratories). Imaging was performed on a Keyence BZ-X800 fluorescence microscope at 40× magnification. Nine adjacent fields-of-view were captured per well and stitched together using a Fiji/ImageJ software plugin. Foci analysis was performed using Focinator v2 software.

Cell Cycle Analysis. Cell cycle analysis was performed using integrated Hoechst nucleic acid dye fluorescence. Briefly, integrated Hoechst fluorescence intensity was log 2 transformed and histograms from DMSO-treated cells were used to identify the centers of the 2N and 4N DNA peaks. These values were used to normalize the 2N DNA peak to 1 and the 4N DNA peak to 2. Cells were then classified by normalized log 2 DNA content as GI (0.75-1.25), S (1.25-1.75), or G2 (1.75-2.5) phase cells. The percentage of cells within each phase of the cell cycle was determined for each treatment condition. The three sets of Hoechst-stained cells corresponding to the three separate DNA foci stains were treated as three independent analyses.

Micronuclei Analysis. An automated image analysis pipeline was developed by YCMD using InCell Analyzer software to quantify micronuclei formation. Nuclei and micronuclei were segmented based on Hoechst nucleic acid dye staining channel. A perinuclear margin was applied around the nuclei to approximate the extent of the cytoplasm and identify micronuclei associated with the parent nucleus. Cells with nuclei associated with at least 1 micronucleus were considered positive.

Statistical analysis. Statistical analysis was performed using GraphPad Prism software. Data are presented as mean or median±SD or SEM as indicated. For in vitro short-term growth delay experiments, IC₅₀ values were determined from the nonlinear regression equation, [inhibitor] vs normalized response with variable slope. For micronuclei assays, comparisons were made with one-way ANOVA and Sidak correction for multiple comparisons. For xenograft growth delay experiments, comparisons were made with Mann-Whitney test (for comparison of 2 groups) or Kruskal-Wallis test with FDR-adjusted p-values with Q set to 5% (for comparison of ≥3 groups). For xenograft survival analysis, Kaplan-Meier analysis was used to evaluate survival rate based on death or removal from study when body weight loss exceeded 20% of initial body weight

### Mouse Protocols

Animals. All animal use was in accordance with the guidelines of the Animal Care and Use Committee (IACUC) of Yale University and conformed to the recommendations in the Guide for the Care and Use of Laboratory Animals (Institute of Laboratory Animal Resources, National Research Council, National Academy of Sciences, 1996).

Mouse Protocols for Flank Studies. A mouse tumor model was established by subcutaneously implanting human LN229 (MGMT−/MMR+) or LN229 (MGMT−/MMR−) cells. Cells were cultured as a monolayer in DMEM+10% FBS (Thermo Fisher) at 37° C. in a humidified atmosphere with 5% CO₂ and passaged between one and three days prior to implantation and media was replaced every 2-3 days as needed to maintain cell viability. Cells were not allowed to exceed 80% confluency. On the day of implantation, cells were trypsinized, washed with complete media and pelleted by centrifugation at 1200 rpm for 5 minutes. The supernatant was decanted, and cells were washed three times with sterile PBS and pelleted by centrifugation. During the final centrifugation, viability was determined using trypan blue exclusion. Cells were resuspended in sterile PBS and diluted 1:1 in Matrigel (Corning, Cat #47743-716) for a final concentration of 5×10⁶ cells/100 μL. 5 million cells were injected into the flank of female nude mice (Envigo, Hsd:Athymic Nude-Foxn1^{nu}, 3-4 weeks age, 15 g). Once tumors reached a minimum volume of 100 mm³, mice were randomized and administered either KL-50 (4a; 5 mg/kg MWF×3 weeks), TMZ (1a; 5 mg/kg MWF×3 weeks), or vehicle (10% cyclodextrin) by oral gavage. Caliper measurements were obtained during the dosing period and at least two weeks following treatment. Mice were euthanized if body weight loss exceeded 20% or if tumor volume increased to greater than 2000 mm³. Kaplan-Meier analysis was used to evaluate survival rate based on death or removal from study.

In a second study, mice were randomized and administered either KL-50 (4a) or vehicle (10% cyclodextrin) by oral gavage or intraperitoneal injection on either M-F×1 or MWF×3 cycles at 5, 15, or 25 mgs/kg. Caliper measurements were obtained during the dosing period and at least two weeks following treatment. Mice were euthanized if body weight loss exceeded 20% or if tumor volume increased to greater than 2000 mm³.

The third study involved MGMT−/MMR+ and MGMT−/MSH6− (shMSH6) LN229 cells. Mice tumors were allowed to grow to a larger average starting volume of ˜350 mm³ before they were randomized and administered either KL-50 (4a; 25 mg/kg MWF×3 weeks) or vehicle (10% cyclodextrin) by oral gavage. Caliper measurements were obtained during the dosing period and at least two weeks following treatment. Mice were euthanized if body weight loss exceeded 20% or if tumor volume increased to greater than 3000 mm³.

Mouse Protocol for Intracranial Study. LN229 MGMT−/MMR− cells stably expressing firefly luciferase (lentivirus-plasmids from Cellomics Technology; PLV-10003), were injected intracranially using a stereotactic injector. Briefly, 1.5 million cells in 5 μl PBS were injected into the brain and the mice were imaged weekly using the IVIS Spectrum In Vivo Imaging System (PerkinElmer) according to the manufacturer's protocol. Images were taken on a weekly basis and acquired 10 min post intraperitoneal injection with d-luciferin (150 mg/kg of animal mass). Tumors were allowed to grow to an average of 1.0×10⁸ RLU before randomization and treated with 5 continuous days of P.O treatment with 10% cyclodextrin vehicle control, TMZ (1a, 25 mg/kg M-F×1 week) or KL-50 (4a, 25 mg/kg M-F×1 week). Quantification of BLI flux (photons/sec) was made through the identification of a region of interest (ROI) for each tumor.

### Example 1

### Synthesis of KL-50 (4a)

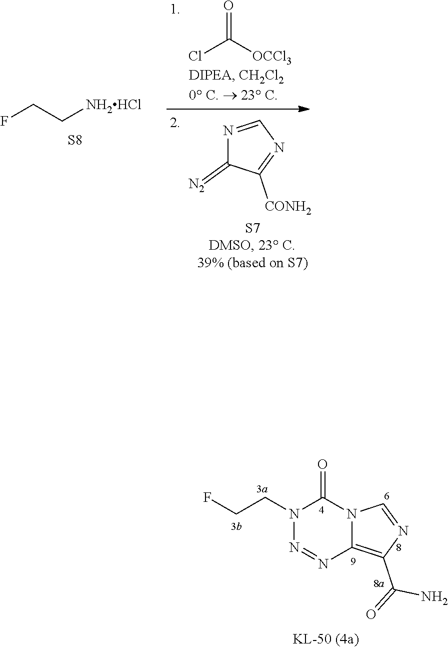

A mixture of fluoroethylamine hydrochloride (3.32 g, 33.3 mmol, 1 equiv), and N,N-di-iso-propyl ethylamine (12.2 mL, 70.0 mmol, 2.10 equiv) in dichloromethane (80 mL) was added dropwise via syringe pump over 45 min to a solution of diphosgene (2.40 mL, 20.0 mmol, 0.60 equiv) in dichloromethane (80 mL) at 0° C. (CAUTION. Gas evolution!). Upon completion of the addition, the cooling bath was removed, and the reaction mixture was allowed to warm to 23° C. over 15 min. The warmed product mixture was immediately transferred to a separatory funnel. The organic layer was washed sequentially with 1 N aqueous hydrochloric acid solution (100 mL, precooled to 0° C.) and saturated aqueous sodium chloride solution (100 mL, precooled to 0° C.). The washed organic layer was dried over magnesium sulfate. The dried solution was filtered, and the filtrate was concentrated (330 mTorr, 31° C.). The unpurified isocyanate so obtained was used directly in the following step.

The unpurified isocyanate obtained in the preceding step (nominally 16.7 mmol, 1.75 equiv) was added dropwise via syringe to a solution of the diazonium S7 (1.31 g, 9.54 mmol, 1 equiv) in dimethyl sulfoxide (10 mL) at 23° C. Upon completion of the addition, the reaction vessel was covered with aluminum foil. The reaction mixture was stirred for 16 h at 23° C. The product mixture was concentrated under a stream of nitrogen. The residue obtained was suspended in dichloromethane and purified by automated flash-column chromatography (eluting with 100% dichloromethane initially, grading to 5% methanol-dichloromethane, linear gradient) to provide KL-50 (4a) as a white crystalline powder (840 mg, 39% based on the diazonium S7). ¹H NMR (400 MHz, DMSO-d₆) δ 8.85 (s, 1H, H₆), 7.83 (s, 1H, NH), 7.70 (s, 1H, NH), 4.82 (dt, J=47.0, 4.9 Hz, 2H, H_{3b}), 4.62 (dt, J=26.0, 4.7 15 Hz, 2H, H₃ₐ). ¹³C NMR (151 MHz, DMSO-d₆) δ 161.5 (C₈ₐ), 139.2 (C₄), 134.2 (C₉), 131.0 (C₈), 128.9 (C₆), 80.8 (d, J=168.7 Hz, C_{3b}), 49.1 (d, J=20.8 Hz, C₃ₐ). ¹⁹F NMR (376 MHz, DMSO-d₆) δ −222.66 (tt, J=47.0, 26.1 Hz). IR (ATR-FTIR), cm⁻¹: 3459 (w), 3119 (m), 1736 (s), 1675 (s). HRMS-ESI (m/z): [M+H]⁺ calcd for [C₇H₈FN₆O₂]⁺ 227.0688, found 227.0676.

### Synthesis of the Imidazolyl Triazene 10:

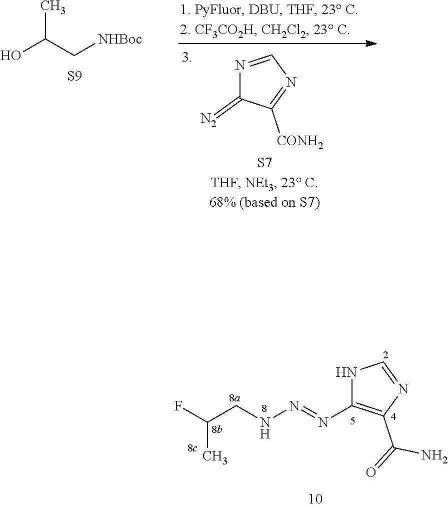

Tert-butyl (2-hydroxypropyl)carbamate (1.72 mL, 10.0 mmol, 1 equiv) was added dropwise via syringe to a mixture of PyFluor (1.77 g, 11.0 mmol, 1.10 equiv) in tetrahydrofuran (10 mL) at 23° C. 1,8-Diazabicyclo(5.4.0)undec-7-ene (3.00 mL, 20.0 mmol, 2.00 equiv) was immediately added dropwise and the reaction mixture was stirred for 48 h at 23° C. under ambient atmosphere. The product mixture was diluted with water (15 mL) and the resulting biphasic mixture was transferred to a separatory funnel. The layers that formed were separated and the aqueous layer was extracted with ethyl acetate (2×15 mL). The organic layers were combined and the combined organic layer was washed sequentially with 1 N aqueous hydrochloric acid solution (2×25 mL) and saturated aqueous sodium chloride solution (2×25 mL). The washed organic layer was dried over sodium sulfate. The dried solution was then filtered and the filtrate concentrated to provide tert-butyl (2-fluoropropyl)carbamate as a clear colorless oil.

The unpurified product obtained in the preceding step (nominally 6 mmol, 1 equiv) was added to a mixture of dichloromethane (30 mL) and trifluoroacetic acid (10 mL) at 23° C. The reaction mixture was stirred for 12 h at 23° C. under ambient atmosphere. The product mixture was concentrated to provide 2-fluoropropylamine trifluoroacetic acid as an opaque oil with excess equivalents of trifluoroacetic acid. The unpurified product obtained in this way (nominally 6 mmol) was dissolved in tetrahydrofuran (10 mL) to generate a working nominal 0.6 M solution for future reactions.

A solution of 2-fluoropropylamine trifluoroacetic acid in tetrahydrofuran (4.40 mL, 2.64 mmol, 1.05 equiv) and triethylamine (1.40 mL, 10 mmol, 4.00 equiv) were added sequentially dropwise via syringe to a suspension of the diazonium S7 (343 mg, 2.50 mmol, 1 equiv) in tetrahydrofuran (15 mL) at 23° C. The reaction mixture was stirred for 6 h at 23° C. The precipitate that formed was collected by vacuum filtration. The precipitate was washed sequentially with ethyl acetate (2×15 mL) and diethyl ether (2×15 mL). The washed precipitate was dried in vacuo to afford the imidazolyl triazene 10 as a light tan powder (365 mg, 68%, based on the diazonium S7). ¹H NMR (600 MHz, DMSO-d₆) δ 12.65 (s, 1H, NH), 10.92 (s, 1H, H₈), 7.54 (s, 1H, H₂), 7.45 (br s, 1H, NH), 7.21 (s, 1H, NH), 4.98 (br d, J=49 Hz, 1H, H_{8b}), 3.87-3.55 (m, 2H, H₈ₐ), 1.34 (dd, J=23.9, 6.3 Hz, 3H, H_{8c}). ¹³C NMR (151 MHz, DMSO-d₆) δ 161.0 (C_{quat.}), 149.5-148.9 (br s, C_{quat.})^{a}, 135.6 (C₂), 115.9 (C_{quat.}), 87.1 (d, J=166.6 Hz, C_{8b}), 48.1 (d, J=22.3 Hz, C₈ₐ), 18.8 (d, J=21.7 Hz, C_{8c}). ¹⁹F NMR (376 MHz, DMSO-d₆) δ −174.43 (dq, J=47.7, 23.9 Hz). IR (ATR-FTIR), cm⁻¹: 3480 (w), 3249 (m), 3077 (m), 1638 (s), 1590 (s), 1427 (s), 1397 (s). HRMS-ESI (m/z): [M+H]⁺ calcd for [C₇H₁₂FN₆O]⁺ 215.1052, found 215.1048. Note: ^{a}Broad peak tentatively attributed to a quaternary carbon from the imidazolyl triazene 10.

### Synthesis of Imidazolyl Triazene 11:

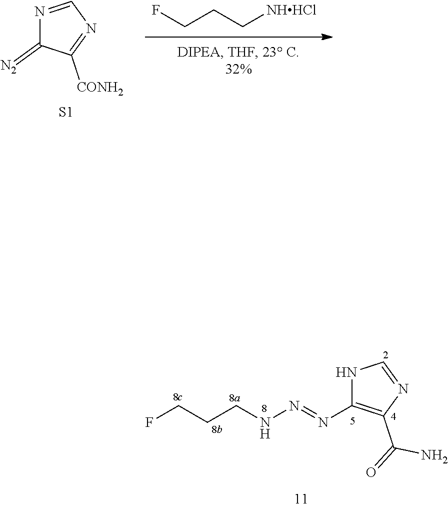

N,N-Di-iso-propyl ethylamine (834 μL, 4.55 mmol, 1.25 equiv) was added dropwise via syringe to a mixture of (3-fluoropropyl)amine hydrochloride (410 mg, 3.65 mmol, 1 equiv) and the diazonium S7 (500 mg, 3.65 mmol, 1 equiv) in tetrahydrofuran (25 mL) at 23° C. The reaction mixture was stirred for 6 h at 23° C. The precipitate that formed was collected by vacuum filtration. The precipitate was washed sequentially with ethyl acetate (2×15 mL) and ether (2×15 mL). The washed precipitate was dried in vacuo to afford the imidazolyl triazene 11 as a light tan powder (251 mg, 32%). ¹H NMR (400 MHz, DMSO-d₆) δ 12.63 (s, 1H, NH), 10.72 (s, 1H, H₈), 7.54 (s, 1H, H₂), 7.46 (s, 1H, NH), 7.28 (s, 1H, NH), 4.54 (dt, J=47.3, 5.8 Hz, 2H, H_{8c}), 3.60-3.50 (m, 2H, H_{8b}), 2.08-1.94 (m, 2H, H_{8af}). ¹³C NMR (151 MHz, DMSO-d₆) δ 161.3 (C_{quat.}), 155.4 (C_{quat.}), 149.7 (C_{quat.}), 135.9 (C₂)^{a}, 81.9 (d, J=161.4 Hz, C_{8c}), 39.6 (C_{8b})^{b}, 26.7 (d, J=19.9 Hz, C₈ₐ). ¹⁹F NMR (376 MHz, DMSO-d₆) δ −219.23 (tt, J=47.1, 26.1 Hz). IR (ATR-FTIR), cm⁻¹: 3483 (w), 3269 (m), 3082 (m), 1640 (m), 1587 (m), 1392 (m). HRMS-ESI (m/z): [M+Na]⁺ calcd for [C₇H₁₁FN₆NaO]⁺ 237.0871, found 237.0986. Notes: ^{a}Signal not observed in 1-D ¹³C NMR spectrum; shift obtained from weak correlation in ¹H-¹³C HSQC spectrum. ^{b}Signal obscured by solvent peak in 1-D ¹³C NMR spectrum; shift obtained from correlation in ¹H-¹³C HSQC spectrum. Notes: ^{b}Signal not observed in 1-D ¹³C NMR spectrum; shift obtained from weak correlation in ¹H-¹³C HSQC spectrum. ^{b}Signal obscured by solvent peak in 1-D ¹³C NMR spectrum; shift obtained from correlation in ¹H-¹³C HSQC spectrum.

### Example 2

Single crystals of KL-50 (4a) suitable for X-ray analysis were obtained by vapor diffusion of dry benzene (3 mL, precipitating solvent) into a syringe filtered (Millipore Sigma, 0.22 μm, hydrophilic polyvinylidene fluoride, 33 mm, gamma sterilized, catalogue number SLGV033RS) solution of KL-50 (4a) (3.6 mg) in dry dichloromethane (3 mL, solubilizing solvent) at 23° C. This yielded two polymorphs of KL-50 (4a) designated Polymorph I (P2₁/n space group, CCDC number 2122008) and Polymorph II (Cc space group, CCDC number 2122009).

### Experimental Procedure for Polymorph I of KL-50 (4a):

Low-temperature diffraction data (ω-scans) were collected on a Rigaku MicroMax-007HF diffractometer coupled to a Dectris Pilatus3R detector with Mo Kα (λ=0.71073 Å) for the structure of 007c-21083. The diffraction images were processed and scaled using Rigaku Oxford Diffraction software (CrysAlisPro; Rigaku OD: The Woodlands, TX, 2015). The structure was solved with SHELXT and was refined against F² on all data by full-matrix least squares with SHELXL (Sheldrick, G. M. Acta Cryst. 2008, A64, 112-122). All non-hydrogen atoms were refined anisotropically. Hydrogen atoms were included in the model at geometrically calculated positions and refined using a riding model. The isotropic displacement parameters of all hydrogen atoms were fixed to 1.2 times the U value of the atoms to which they are linked (1.5 times for methyl groups). The full numbering scheme of compound 007c-21083 can be found in the full details of the X-ray structure determination (CIF), which is included as Supporting Information. CCDC number 2122008 (007c-21083) contains the supplementary crystallographic data (FIG. ZS11).

TABLE 2
Crystal data and structure refinement for Polymorph I of KL-50 (4a):

| Identification code | 007c-21083 | |
|---|---|---|
| Empirical formula | C7 H7 F N6 O2 | |
| Formula weight | 226.19 | |
| Temperature | 93(2) | K |
| Wavelength | 0.71073 | Å |
| Crystal system | Monoclinic | |
| Space group | P21/n | |
| Unit cell dimensions | a = 7.1861(5) | Å |
| | b = 7.6918(5) | Å |
| | c = 16.4546(12) | Å |
| Volume | 903.19(11) | Å³ |
| Z | 4 | |
| Density (calculated) | 1.663 | Mg/m³ |
| Absorption coefficient | 0.141 | mm⁻¹ |
| F(000) | 464 | |
| Crystal size | 0.200 × 0.200 × 0.020 | mm³ |

| | |
|---|---|
| Crystal color and habit | Colorless Plate |
| Diffractometer | Dectris Pilatus 3R |
| Theta range for data collection | 2.927 to 31.467°. |
| Index ranges | −9 <= h <= 10, −10 <= k <= 9, −23 <= 1 <= 19 |
| Reflections collected | 9836 |
| Independent reflections | 2539 [R(int) = 0.0300] |
| Observed reflections (I > 2sigma(I)) | 2135 |
| Completeness to theta = 25.242° | 99.9% |
| Absorption correction | Semi-empirical from equivalents |
| Max. and min. transmission | 1.00000 and 0.31047 |
| Solution method | SHELXT-2014/5 (Sheldrick, 2014) |
| Refinement method | SHELXL-2014/7 (Sheldrick, 2014) |
| Data/restraints/parameters | 2539/0/145 |
| Goodness-of-fit on F² | 1.050 |
| Final R indices [I > 2sigma(I)] | R1 = 0.0358, wR2= 0.0885 |
| R indices (all data) | R1 = 0.0450, wR2 = 0.0929 |
| Largest diff. peak and hole | 0.360 and −0.250 e · Å⁻³ |

### Example 3

### Experimental Procedure for Polymorph II of KL-50 (4a):

Low-temperature diffraction data (ω-scans) were collected on a Rigaku MicroMax-007HF diffractometer coupled to a Satumn994+ CCD detector with Cu Kα (λ=1.54178 Å) for the structure of 007b-21124. The diffraction images were processed and scaled using Rigaku Oxford Diffraction software (CrysAlisPro; Rigaku OD: The Woodlands, TX, 2015). The structure was solved with SHELXT and was refined against F² on all data by full-matrix least squares with SHELXL (Sheldrick, G. M. Acta Cryst. 2008, A64, 112-122). All non-hydrogen atoms were refined anisotropically. Hydrogen atoms were included in the model at geometrically calculated positions and refined using a riding model. The isotropic displacement parameters of all hydrogen atoms were fixed to 1.2 times the U value of the atoms to which they are linked (1.5 times for methyl groups). The full numbering scheme of compound 007b-21124 can be found in the full details of the X-ray structure determination (CIF), which is included as Supporting Information. CCDC number 2122009 (007b-21124) contains the supplementary crystallographic data.

TABLE 3
Crystal data and structure refinement for Polymorph II of KL-50 (4a):

| Identification code | 007b-21124 | |
|---|---|---|
| Empirical formula | C7 H7 F N6 O2 | |
| Formula weight | 226.19 | |
| Temperature | 93(2) | K |
| Wavelength | 1.54184 | Å |
| Crystal system | Monoclinic | |
| Space group | Cc | |
| Unit cell dimensions | a = 6.6061(2) | Å |
| | b = 23.1652(6) | Å |
| | c = 11.9879(3) | Å |
| Volume | 1824.52(9) | Å³ |
| Z | 8 | |
| Density (calculated) | 1.647 | Mg/m³ |
| Absorption coefficient | 1.218 | mm⁻¹ |
| F(000) | 928 | |
| Crystal size | 0.200 × 0.020 × 0.020 | mm³ |

| | |
|---|---|
| Crystal color and habit | Colorless Needle |
| Diffractometer | Rigaku Saturn 944 + CCD |
| Theta range for data collection | 3.816 to 66.573°. |
| Index ranges | −7 <= h <= 7, −27 <= k <= 27, −14 <= 1 <= 14 |
| Reflections collected | 31349 |
| Independent reflections | 3204 [R(int) = 0.0818] |
| Observed reflections (I > 2sigma(I)) | 2950 |
| Completeness to theta = 66.573° | 100.0% |
| Absorption correction | Semi-empirical from equivalents |
| Max. and min. transmission | 1.00000 and 0.59718 |
| Solution method | SHELXT-2014/5 (Sheldrick, 2014) |
| Refinement method | SHELXL-2014/7 (Sheldrick, 2014) |
| Data/restraints/parameters | 3204/2/291 |
| Goodness-of-fit on F² | 1.041 |
| Final R indices [>2sigma(I)] | R1 = 0.0330, wR2 = 0.0770 |
| R indices (all data) | R1 = 0.0381, wR2 = 0.0795 |
| Absolute structure parameter | 0.1(2) |

### Example 4

We synthesized the imidazotetrazine 4a (KL-50) and the triazene 4b (KL-85), as vehicles to deliver 2-fluoroethyl diazonium (4c), and a series of related agents to probe structure-activity relationships in tissue culture (FIG. Z1, D and F). The 2-fluoropropyl- and 3-fluoropropyl-triazenes (10 and 11, respectively) were prepared by diazotization of 4-aminoimdazole-5-carboxamide, followed by the addition of the respective amine. All other triazenes were prepared according to literature procedures. We evaluated the cytotoxicity of our compounds in short-term cell viability assays against four isogenic LN229 glioblastoma cell lines engineered to be proficient or deficient in MGMT and/or MMR activity, using short hairpin RNAs (shRNAs) targeting MSH2 (referred to as MGMT+/−, MMR+/− cells hereafter; FIG. ZS2A). This approach allowed us to rapidly and rigorously interrogate the relationship between MGMT and MMR status and compound activity.

### Example 5

The IC₅₀ values of these agents are shown in Table 1 (FIG. Z2A) and representative dose-response curves are shown in FIG. Z2B (additional data are presented in FIG. ZS2, B to G). KL-85 (4b) retained potency in MGMT−/MMR− cells (IC₅₀=27.5 μM), while TMZ (1a) was essentially inactive (IC₅₀=837.7 μM). Structure-activity studies were consistent with the mechanistic pathway shown in FIG. Z1E. The 2,2-difluoroethyl triazene 9 and the 2-fluoropropyl triazene 10 possessed reduced potency in MGMT−/MMR− cells (FIG. ZS2, B and C), in agreement with the reduced rates of displacement following introduction of an additional fluorine or alkyl substituent. The 2-chloroethyl triazene 12b was modestly potent but not as selective for MGMT− cell lines (FIG. ZS2E) which likely derives from faster, non-selective ICL formation arising from chloride displacement (vide infra). The 3-fluoropropyl triazene 11 demonstrated low activity in all four cell lines, presumably due to inefficient transfer of the electrophile to DNA (FIG. ZS2D). The ethyl triazene 13 also demonstrated low activity (FIG. ZS2F). This compound may undergo rapid elimination to ethylene gas following conversion to ethyl diazonium.

We prepared KL-50 (4a) by diazotization of 4-aminoimidazole-5-carboxamide followed by the addition of (2-fluoroethyl)isocyanate (39% overall yield). The potency of 4a mirrored that of 4b in the four cell lines examined (FIG. Z2B). To benchmark selectivity, we evaluated the experimental agent mitozolomide (MTZ, 12a) and the clinical nitrosourea lomustine (aka CCNU, 14), which have been studied with hopes of addressing TMZ (1a) resistance. However, these agents were only ˜4-7-fold selective for MGMT-deficient cells (FIG. ZS2G and FIG. Z2B, respectively), as opposed to the ˜25-fold selectivity seen with KL-50 (4a).

### Example 6

We validated the antitumor activity of KL-50 (4a) in clonogenic survival assays (CSAs) and additional cell lines in vitro. TMZ (1a) possessed negligible activity in MGMT+LN229 cells, irrespective of MMR status, and induced robust tumor cell killing in MGMT−, MMR+ cells that was abolished in isogenic cells lacking MMR (FIG. Z2C). Lomustine (14) was effective in MMR− cells but was cytotoxic to MGMT+ cells (FIG. ZS2H). In contrast, KL-50 (4a) demonstrated robust antitumor activity in MGMT− cells, independent of MMR status, with minimal toxicity to MGMT+ cells at doses up to at least 200 μM (FIG. Z2D). We observed a similar pattern of activities in several unique cell lines across different tumor types with intrinsic or induced loss of MGMT and/or MMR activity. For example, TMZ (1a) was inactive in DLD1 cells, which possess MGMT but lack functional MMR (MSH6−) with or without induced depletion of MGMT using O⁶-benzylguanine (O⁶BG; FIG. Z2E). In contrast, KL-50 (4a) was toxic to these cells, but only after O⁶BG-induced MGMT depletion (FIG. Z2F). TMZ (1a) was inactive in HCT116 colorectal cancer cells, which lack the MMR protein MLH1, regardless of MGMT levels (FIG. Z2G). Restoration of MMR activity via complementation with chromosome 3 containing MLH1 resulted in the enhanced sensitivity to TMZ (1a), which was further potentiated by MGMT depletion (FIG. Z2G). In contrast, KL-50 (4a) induced selective tumor cell killing specifically in the setting of O⁶BG-induced MGMT suppression, in both MLH1-deficient and MLH1-complemented cells (FIG. Z2H). MMR status and O⁶BG-induced loss of MGMT expression was confirmed by western blot analysis (FIG. ZS2, I and J). We also confirmed the activity of KL-50 (4a) in MGMT− LN229 cells engineered to lack expression of other key MMR proteins including MSH6, MLH1, PMS2, and MSH3 (FIG. ZS3). Finally, we compared the cytotoxicity of KL-50 (4a) and TMZ (1a) in normal human fibroblast cells and observed no increase in toxicity with KL-50 (4a) (FIG. ZS2K). These data define KL-50 (4a) as a first-in-class molecule that overcomes MMR mutation-induced resistance while retaining selectivity for tumor cells lacking MGMT.

### Example 7

We used a well-established comet assay adapted for ICL detection to determine if ICLs were formed in MGMT− cells treated with KL-50 (4a) (FIG. Z3, A and B). In this assay, cells were sequentially exposed to genotoxins and ionizing radiation, and then analyzed by single cell alkaline gel electrophoresis. Attenuation of the IR-induced comet tail is indicative of ICL formation. In the absence of IR, TMZ (1a, 200 μM) and KL-50 (4a, 200 μM) both induced tailing in MGMT−/MMR+ cells, while mitomycin C (MMC, 0.1 or 50 μM) did not. Exposure to 50 μM MMC for 2 h completely abolished the IR-induced comet tail, whereas exposure to 0.1 μM MMC (chosen to be ˜10-fold greater than the IC₅₀ for this drug, comparable to 200 μM KL-50 (4a) or TMZ (1a)) for 24 h caused a partial reduction in the IR-induced comet tail. TMZ (1a, 200 μM) did not reduce DNA migration following IR, in agreement with its known function as a monoalkylation agent with no known crosslinking activity. In contrast, KL-50 (4a, 200 μM) reduced the % DNA in the tail to levels similar to those seen for 0.1 μM MMC. We observed a similar pattern of comet tail migration for MMC and KL-50 (4a) in MGMT−/MMR− cells, which supports an MMR-independent crosslinking mechanism. Comparable results were observed in MGMT−/MMR+ cells treated with KL-85 (4b) (FIG. ZS4, A and B).

We carried out this assay at varying time points (2-24 h) to assess the rates of ICL formation in MGMT−/MMR− cells treated with KL-50 (4a), MTZ (12a), or TMZ (1a) (FIG. Z3, C and D). The chloroethyl derivative MTZ (12a) reduced DNA mobility within 2 h, consistent with the cell line selectivities above and literature reports that this agent rapidly forms ICLs by chloride displacement from other sites of alkylation. TMZ (1a) did not induce a statistically significant decrease in DNA migration within 24 h. However, we observed a time-dependent decrease in DNA mobility in cells treated with KL-50 (4a), with the largest difference observed between 8 and 24 h, consistent with the reported half-life of O⁶FEtG (6; 18.5 h). In the unirradiated samples, KL-50 (4a), MTZ (12a), and TMZ (1a) all induced maximal damage at 2 h, which decreased over time, consistent with progressive DNA repair (FIG. ZS4, C and D). Analysis of genomic DNA isolated from LN229 MGMT−/MMR+ cells treated with KL-50 (4a, 200 μM) or KL-85 (4b, 200 μM) by denaturing gel electrophoresis demonstrated the presence of crosslinked DNA (FIG. Z3E). TMZ (1a) and MTIC (1b) showed no evidence of ICL induction. Similarly, linearized pUC19 plasmid DNA treated with KL-50 (4a, 100 μM) also possessed ICLs, with delayed rates of formation relative to 12b (FIG. Z3F). Collectively, these data support a mechanism of action for KL-50 (4a) involving the slow generation of DNA ICLs in the absence of MGMT.

### Example 8

We then probed for alternative mechanisms of action implicating nucleotide excision repair (NER), base excision repair (BER), reactive oxygen species (ROS), and DNA duplex destabilization. Short term cell viability assays in isogenic mouse embryonic fibroblasts (MEFs) proficient or deficient in XPA, a common shared NER factor, revealed no differential sensitivity, either with or without O⁶BG-induced MGMT depletion (FIG. ZS5A). N7MeG lesions induced by TMZ (1a) are prone to spontaneous depurination, apurinic (AP) site formation, and single strand breaks (SSBs), which are all known BER substrates. To probe for potential differential induction of BER substrates by KL-50 (4a) compared to TMZ (1a), we performed in vitro supercoiled plasmid DNA assays that measure the formation of AP sites. We observed similar levels of spontaneous and enzyme-catalyzed SSBs from AP sites with KL-50 (4a) and TMZ (1a), suggesting comparable levels of depurination (FIG. ZS5B). Co-treatment with increasing concentrations of the ROS scavenger N-acetylcysteine (NAC) did not rescue cell viability (FIG. ZS5C). Melting point analysis did not reveal any notable differences in DNA stability resulting from fluoroethylation compared to methylation (FIG. ZS5D). These data suggest that NER status, AP site induction, ROS, and altered DNA stability are peripheral or noncontributory to the effectiveness of KL-50 (4a).

We characterized the profile of DDR activation across our four isogenic cell lines after treatment with KL-50 (4a) or TMZ (1a). Our prior finding that the ATR-CHK1 signaling axis is activated in response to TMZ (1a)-induced replication stress in MGMT-deficient cells prompted us to analyze the phosphorylation status of CHK1 and CHK2 in LN229 MGMT+/− and MMR+/− cells. KL-50 (4a) induced CHK1 and CHK2 phosphorylation in MGMT− cells regardless of MMR status, whereas TMZ (1a) only induced phospho-CHK1 and —CHK2 in MGMT−/MMR+ cells (FIG. ZS6A). We analyzed foci formation of the DDR factors phospho-SER139-H2AX (γH2AX), p53 binding protein 1 (53BP1), and phospho-SER33-RPA2 (pRPA) over the period of 2 to 48 h (FIG. Z4, A to D, and FIG. ZS6, B and C). KL-50 (4a) induced a maximal foci response at 48 h, specifically in MGMT− cells and irrespective of MMR status (4a). TMZ (1a) induced a comparable response in MGMT− cells, but this was abolished in the absence of functional MMR, consistent with known MMR-silencing-based resistance. We observed a reduced level of foci formation in MGMT+/MMR+ cells that was absent in MGMT+/MMR− cells, suggesting an MMR-dependent DNA damage response in these cells. However, these foci dissipate at later timepoints (72-96 h; FIG. ZS6D), and they are not associated with appreciable cellular toxicity (as shown earlier in FIG. Z2, C and D).

KL-50 (4a) induced increasing G2 arrest on progression from 24 to 48 h in MGMT−/MMR+ cells, as determined by simultaneous analysis of DNA content based on nuclear (Hoechst) staining in the foci studies above (FIG. Z4E and FIG. ZS7, A and B). KL-50 (4a) induced an attenuated G2 arrest in MGMT−/MMR− cells, consistent with a role of MMR in the G2-checkpoint. This effect in MGMT−/MMR− cells was absent following TMZ (1a) treatment. Both TMZ (1a) and KL-50 (4a) induced a moderate G2 arrest in MGMT+/MMR+ cells.

We quantified the levels of DDR foci across the individual cell cycle phases (FIG. ZS8). KL-50 (4a) induced foci formation primarily in the S- and G2-phases of the cell cycle, which is consistent with replication blocking by ICLs. Foci increased in MGMT-G1 cells at 48 h, suggesting that a fraction of cells may progress through S-phase with unrepaired DNA damage. Consistent with this, a significant increase in micronuclei was observed at 48 h following KL-50 (4a) treatment, which was greatest in the MGMT−/MMR− cells (FIG. Z4F and FIG. ZS9, A and B). TMZ (1a) displayed a similar pattern of foci induction in the S- and G2-phases, with smaller increases in G1-phase foci and micronuclei formation at 48 h in MGMT−/MMR+ cells. In contrast, we did not observe foci induction or micronuclei formation in MGMT−/MMR− cells exposed to TMZ (1a). These findings are in agreement with the differential toxicity profiles of KL-50 (4a) and TMZ (1a): KL-50 (4a) induces multiple successive markers of DNA damage and engagement of the DDR in MGMT− cells, independent of MMR status, whereas the effects of TMZ (1a) are similar in MGMT−/MMR+ cells but absent in MMR− cells. Coupled with the ICL kinetics data presented above, these time-course data support a slow rate of ICL induction in situ by KL-50 (4a).

These foci data suggest that KL-50 (4a) induces replication stress (e.g., pRPA foci formation) and DSB formation (e.g., γH2AX and 53BP1 foci, which are known to follow the formation of ICLs). Consistent with this, BRCA2- and FANCD2-deficient cells are hypersensitive to KL-50 (4a; FIG. Z4, G to I, and FIG. ZS9, C to F). In two MGMT-proficient cell models, BRCA2 loss enhanced the toxicity of KL-50 (4a) following MGMT depletion via O⁶BG (FIG. Z4, H and I). We observed FANCD2 ubiquitination by KL-50 (4a) specifically in MGMT− cells, suggesting activation of the Fanconi anemia (FA) ICL repair pathway (FIG. ZS9G). As previously reported, TMZ (1a) also induced FANCD2 ubiquitination but only in MGMT−/MMR+ cells.

We evaluated the activity of KL-50 (4a) and TMZ (1a) in vivo using murine flank tumor models derived from the isogenic LN229 MGMT− cell lines We treated MGMT−/MMR+ and MGMT−/MMR− flank tumors with KL-50 (4a) or TMZ (1a) (5 mg/kg MWF×3 weeks) as previously described for TMZ (1a). TMZ (1a) suppressed tumor growth in the MGMT−/MMR+ tumors (FIG. Z5A). KL-50 (4a) was statistically non-inferior to TMZ (1a), despite a 17% lower molar dosage owing to its higher molecular weight. In the MGMT−/MMR− tumors, TMZ (1a) demonstrated no efficacy, while KL-50 (4a) potently suppressed tumor growth (FIG. Z5B). KL-50 (4a) treatment resulted in no significant changes in body weight compared to TMZ (1a) or control (FIG. Z5C). Representative Kaplan-Meier survival curves are shown in FIG. Z5D with a greater than 5-week increase in median OS for KL-50 (4a) vs TMZ (1a). KL-50 (4a) was effective and non-toxic using different dosing regimens (5 mg/kg, 15 mg/kg, 25 mg/kg), treatment schedules (MWF×3 weeks, M-F×1 week), and routes of drug administration (PO, IP) in mice bearing MGMT−/MMR+ and MGMT−/MMR− flank tumors (FIG. Z5E). KL-50 (4a; 25 mg/kg PO MWF×3 weeks) potently suppressed the growth of large (˜350-400 mm³) MGMT−/MMR+ and MGMT−/MSH6-tumors (FIG. Z5F). KL-50 (4a; 25 mg/kg IP M-F×1 week) was also effective in an orthotropic, intracranial LN229 MGMT−/MMR− model, whereas TMZ (1a) only transiently suppressed tumor growth (FIG. Z6A).

A focused maximum tolerated dose study revealed KL-50 (4a) is well-tolerated. Healthy mice were treated with escalating doses of KL-50 (4a) (0, 25, 50, 100, and 200 mg/kg×1 dose), and monitored over time for changes in both weights and hematologic profiles. Mice in the higher dosage groups (100 or 200 mg/kg) experienced a greater than 10% weight loss after treatment administration, which regressed to baseline at the end of one week (FIG. Z6B). Two of three mice in the 200 mg/kg treatment group became observably ill warranting euthanasia, but no evidence of toxicity was observed in the remaining cohorts. As the main dose limiting systemic toxicity of TMZ (1a) is myelosuppression, we measured complete blood counts for all mice on day 0 before treatment and subsequently on day 7 after drug administration. Overall, neutrophils and lymphocytes experienced the most significant drops in cell count, although all blood counts were within normal physiological ranges (defined as values falling within 2 SDs of the average for healthy mice) for all cohorts (FIG. Z6C). Taken together, these data demonstrate the robust in vivo efficacy, systemic tolerability, and CNS penetrance of KL-50 (4a).

### Example 9

Herein, we described the discovery of novel agents for the eradication of drug-resistant glioma in vitro and in vivo. Without being bound by any particular theory, the success of these agents arises from two factors. First, following on the seminal clinical studies of Stupp and co-workers, who established MGMT expression as a predictive biomarker for TMZ (1a) treatment, we capitalize on MGMT silencing (which occurs in ˜50% of GBMs and ˜70% of grade II/III gliomas) to obtain tumor cell selectivity. Second, and in a departure from prior studies, we utilized bifunctional agents that are specifically designed to evolve slowly to ICLs following transfer to O⁶G, thereby establishing an MMR-independent method to amplify the therapeutic impact of MGMT silencing.

This strategy has led to a new class of agents for treatment of MGMT− glioma independent of MMR status. MMR mutation-induced alkylator resistance has been a major barrier to treatment efficacy, likely since the introduction of TMZ (1a) into glioma treatment regimens in the early 1990s. Bifunctional alkylation agents, such as lomustine (14) and MTZ (12a), have been tested with the hopes of overcoming TMZ (1a) resistance over the last ˜30 years, but these agents lack a therapeutic index owing to their activity in MGMT+(normal tissue) cells.

Literature data supports the notion that the remarkable cell line selectivity of KL-50 (4a) derives strictly from the poor leaving group ability of fluoride. While the aliphatic C—F bond is strong (˜109 kcal/mol) and not normally susceptible to cleavage by bimolecular nucleophilic displacement, the appropriate positioning of hydrogen bond donors or covalently attached nucleophiles can promote substitution. The half-lives of O⁶-(2-fluoroethyl)guanosine (S1) and O⁶-(2-chloroethyl)guanosine (S4) are ˜18.5 h and ˜ 18 min, respectively, at 37° C. and pH 7.4 (FIG. ZS1, A and B). Intramolecular halide displacement gives the common intermediate N1,O⁶-ethanoguanosine (S2) which undergoes ring opening attack by water to yield N1-(2-hydroxyethyl)guanosine (S3). By comparison, attempts to hydrolyze N7-(2-fluoroethyl)guanosine (S5) to N7-(2-hydroxyethyl)guanosine (S6) in aqueous buffer (pH 7) at 37° C. were reportedly unsuccessful, likely due to an inability to form a similar cationic cyclized intermediate (FIG. ZS1C). Thus, while O⁶FEtG (5) lesions likely only constitute a small fraction of alkylation products derived from KL-50 (4a), we hypothesize that the more prevalent sites of alkylation, such as N7G, do not form ICLs at a significant rate, and are readily resolved. The intramolecular displacement pathway (5→6) provides an essential acceleration in the formation of ICLs by KL-50 (4a) to biologically relevant timescales and enables an ample kinetic window for MGMT-mediated repair of the primary O⁶FEtG (5) adduct.

These data also provide an explanation for the failure of related cross-linking agents to display the therapeutic index that underpins TMZ's (1a) success. 2-Chloroethyl nitrosoureas (e.g., lomustine, 14) or 2-chloroethylimidazotetrazines (e.g., MTZ, 12a) are known to form ICLs such as 8 by a pathway analogous to KL-50 (4a, see FIG. Z1E). However, they can also generate ICLs via direct chloride displacement from 2-chlorethyl adducts present at other sites of DNA alkylation, which degrades the therapeutic index of these compounds. Consistent with this, our time-course analysis established a faster onset of ICLs for MTZ (12a) than KL-50 (4a), and, in turn, explains their differential MGMT selectivity (˜7-fold and ˜25-fold for 12a and 4a, respectively).

An order-of-estimate calculation provides insight into the number of ICLs necessarily generated by KL-50 (4a) to induce toxicity. It has been reported that the mean lethal dose of ICLs in HeLa cells is 230 and TMZ (1a) has been demonstrated to yield 20,600 O⁶MeG (3) adducts per cell at a dose of 20 μM. Assuming a similar level of O⁶FEtG (5) lesions are induced by KL-50 (4a) at the IC₅₀ (˜20 μM) in MGMT−/MMR− LN229 cells, the number of adducts required to convert to ICLs to generate the mean lethal dose is ˜1 in 90, or ˜1.1% cross-linking efficiency.

We performed extensive characterization of KL-50 (4a) versus TMZ (1a) activity in vitro to support our hypothesis that we can selectively target MGMT− cells independent of MMR status. While MGMT−/MMR− cells display no signs of DNA damage or DNA repair signaling in response to TMZ (1a), we found robust, MMR-independent, activation of DNA damage checkpoint signaling, DNA repair foci formation, cell cycle arrest, and micronuclei formation following KL-50 (4a) treatment. Moreover, KL-50 (4a) retained its effectiveness in vivo in MMR-deficient flank and intracranial tumor models resistant to TMZ (1a) as well as in large MSH6-deficient tumors, a commonly lost MMR component reported in glioma patients.

Beyond MGMT-silenced recurrent glioma, we anticipate other potential beneficial indications for selective targeting of cancer cells with KL-50 (4a). MGMT silencing has been reported in 40% of colorectal cancers and 25% of non-small cell lung cancer, lymphoma, and head & neck cancers. MGMT mRNA expression is also reduced in subsets of additional cancer types, including breast carcinoma, bladder cancer, and leukemia. MMR loss, as reported by microsatellite instability, is a well-established phenomenon in multiple cancer types and leads to resistance to various standard of care agents. It therefore stands to reason that there are likely other subsets of MGMT−/MMR− tumors in both initial and recurrent settings that would be ideal targets for KL-50 (4a).

Our data also suggest KL-50 (4a) will display a higher therapeutic index in tumors with MGMT deficiency and impaired ICL repair, including HR deficiency. Specifically, we demonstrated that FANCD2- and BRCA2-deficient cells are hypersensitive to KL-50 (4a), particularly in the setting of MGMT depletion. Remarkably, the therapeutic index (TI) of KL-50 (4a) in the DLD1 isogenic model, as measured by the ratio of IC₅₀ values in MGMT+/BRCA2+ cells compared to MGMT−/BRCA2− cells, was ˜600-fold, vastly larger than canonical crosslinking agents such as cisplatin (42-fold) or MMC (26-fold). A similar amplification of the TI was seen in the PEO1/4 model with KL-50 (4a) (62-fold) vs. cisplatin (13-fold) or MMC (7-fold). HR-related gene mutations have been detected in a substantial number of tumors across multiple cancer types (17.4% in 21 cancer lineages) and novel methods have been developed to assess for tumor-associated HR deficiency. Thus, in the modern era of molecular precision medicine, the biomarker-guided use of KL-50 (4a) in individual cancers could result in therapeutic indices and exquisite tumor sensitivities previously only observed with synthetic lethal interactions targeting DNA repair proteins. Finally, we envision many possibilities for combination studies of KL-50 (4a) with DNA repair inhibitors such as checkpoint kinase inhibitors or potentially immunotherapy in the setting of MMR mutations.

We anticipate that these findings may have profound clinical implications for patients with recurrent MGMT-methylated glioma, of which up to half acquire TMZ (1a) resistance via loss of MMR. As demonstrated by our analysis of related TMZ (1a) derivatives, KL-50 (4a) is uniquely designed to fill this therapeutic void. In addition, because KL-50 (4a) may be rapidly phased into clinical trials and readily amenable to derivatization for improved drug pharmacokinetic properties, such enhanced as CNS penetration, based on prior work with the imidazotetrazine scaffold. More broadly, incorporating the rates of DNA modification and DNA repair pathways in therapeutic design strategies may lead to the development of additional selective chemotherapies.

### Example 10: Compound Syntheses

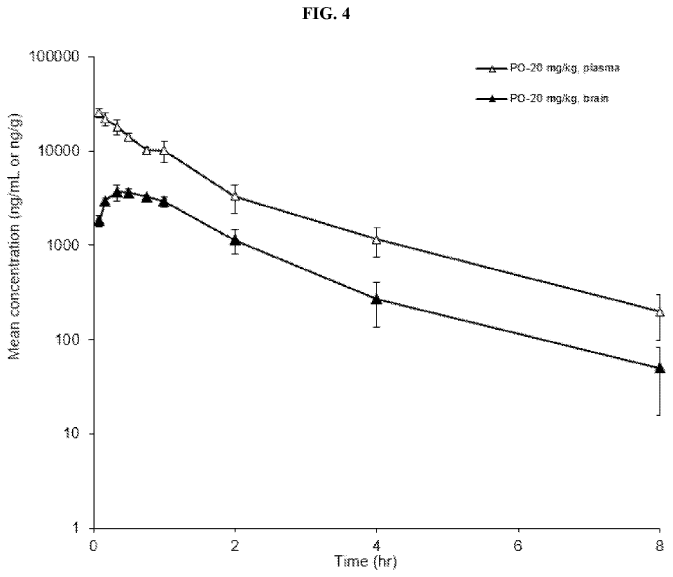

The present compounds may be synthesized according to the scheme of FIG. 2 or as described above (as appropriate). The NMR spectra of KL50 and N-methyl KL50 are shown in FIG. 4-7.

### Example 11: Synergy Between Compounds of the Present Disclosure and ATR Inhibitors

In the absence of MGMT, O6MeG adducts accumulate, but they are insufficient to inhibit DNA replication. Instead, persistent TMZ-induced O6MeG lesions mispair with thymine during DNA replication. This incorrect pairing, in turn, activates the MMR pathway, which attempts to repair these lesions by resecting the newly synthesized DNA strand. However, thymine again is inserted opposite O6MeG, leading to additional MMR cycles. These “futile iterative cycles” trigger apoptosi. New insights have been gained into the mechanistic basis for TMZ sensitivity in MGMT− cells. Engagement of the DDR ataxia-telangieciasia-utiated-and-Rad3-related kinase (ATR) and its major downstream effector checkpoint kinase 1 (Chk1) axis can play a critical role in the exquisite sensitivity that is seen in these tumors. This alternative, or partially parallel, pathway also is outlined herein. The data implicate replication stress as an important mechanism for alkylator sensitivity, and potent synergy between TMZ and ATR inhibitors, specifically in MGMT− cells, was identified. In certain embodiments, synergy is observed with KL-50 and ATR inhibitors. In certain embodiments, the ATR inhibitor is AZ20 ((R)-4-(2-(3H-indol-4-yl)-6-(1-(methylsulfonyl)cyclopropyl)pyrimidin-4-yl)-3-methylmorpholine). In certain embodiments, synergy is observed with administration of a MZT regimen (i.e., minocycline, telmisartan, and zoledronic acid) and an AZT inhibitor.

### Example 12: Testing

The following test protocols were used.

Clonogenic Survival Assay (FIG. 9): Isogenic glioma (Ln 229) cells were pretreated with the test drug in culture for 48-72 hours at the specified dilutions. Cells were then transferred in media without drug to 6-well plates in triplicate at 3-fold dilutions ranging from 9,000 to 37 cells per well. After 14 days, plates were washed with PBS and stained with crystal violet. Colonies were counted by hand. Counts were normalized to plating efficiency of the corresponding treatment condition.

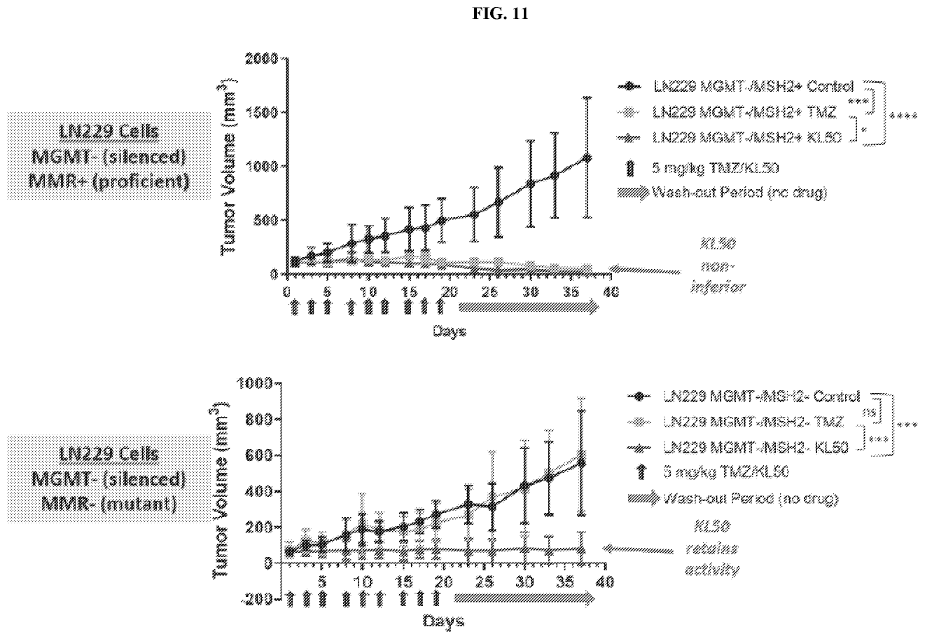

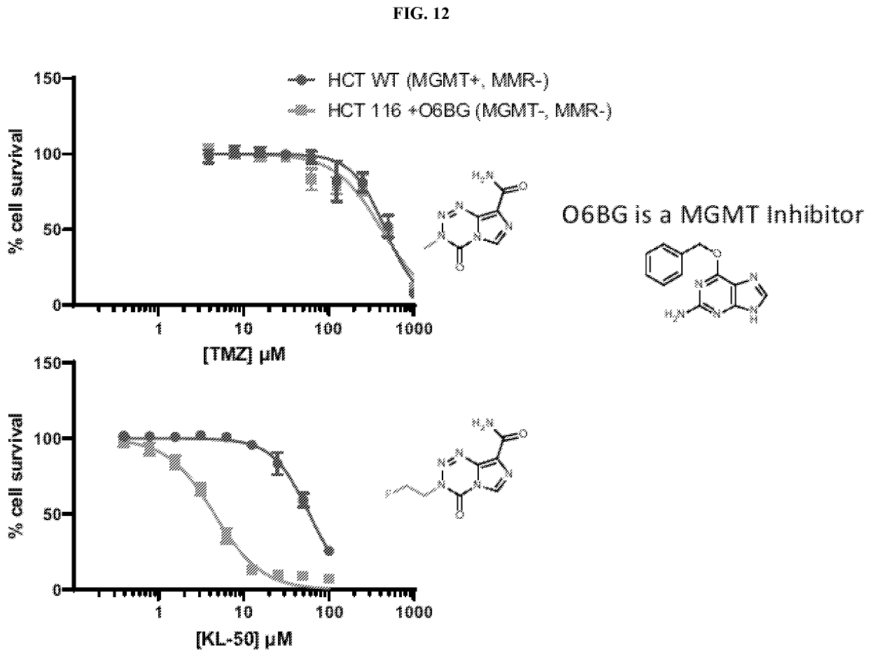

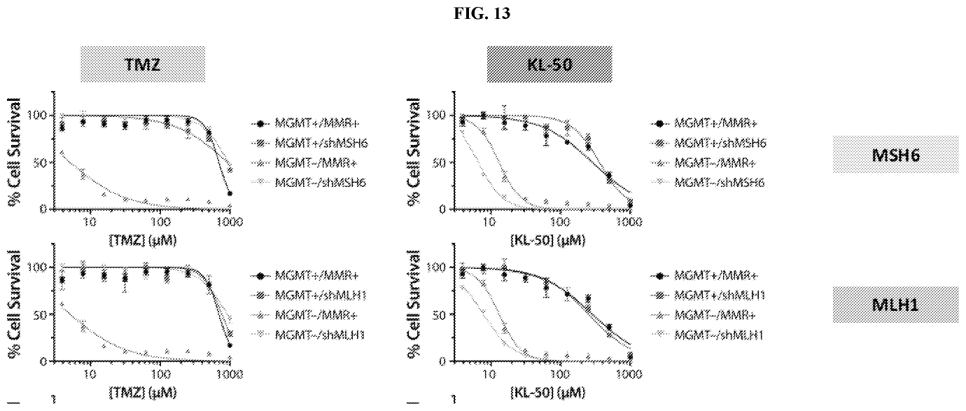

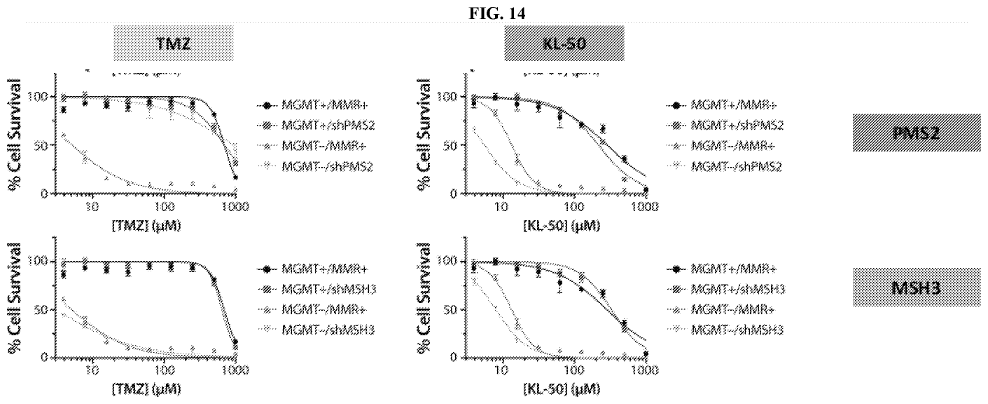

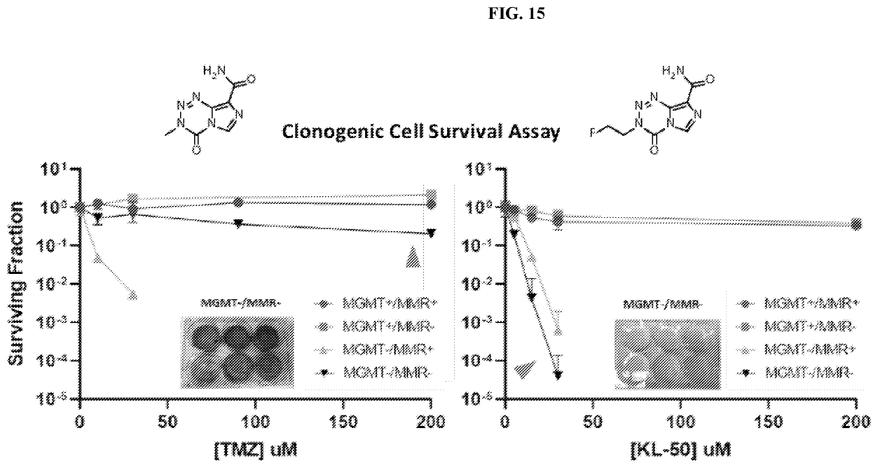

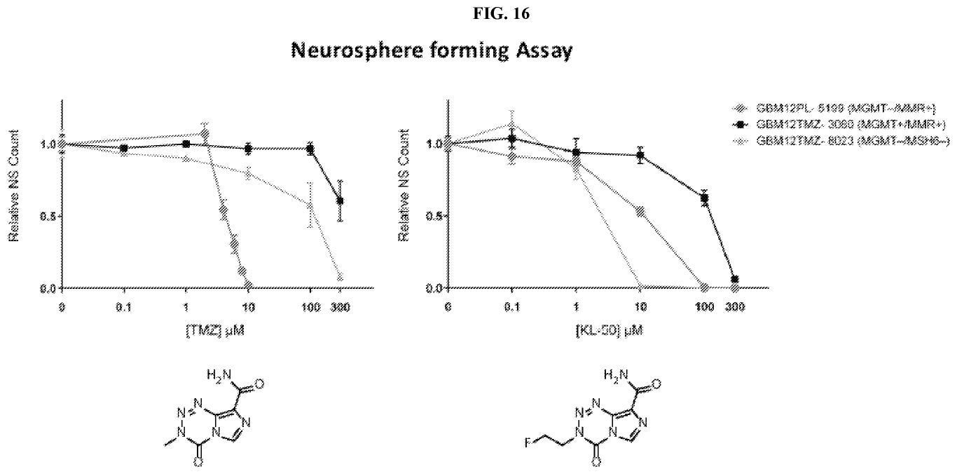

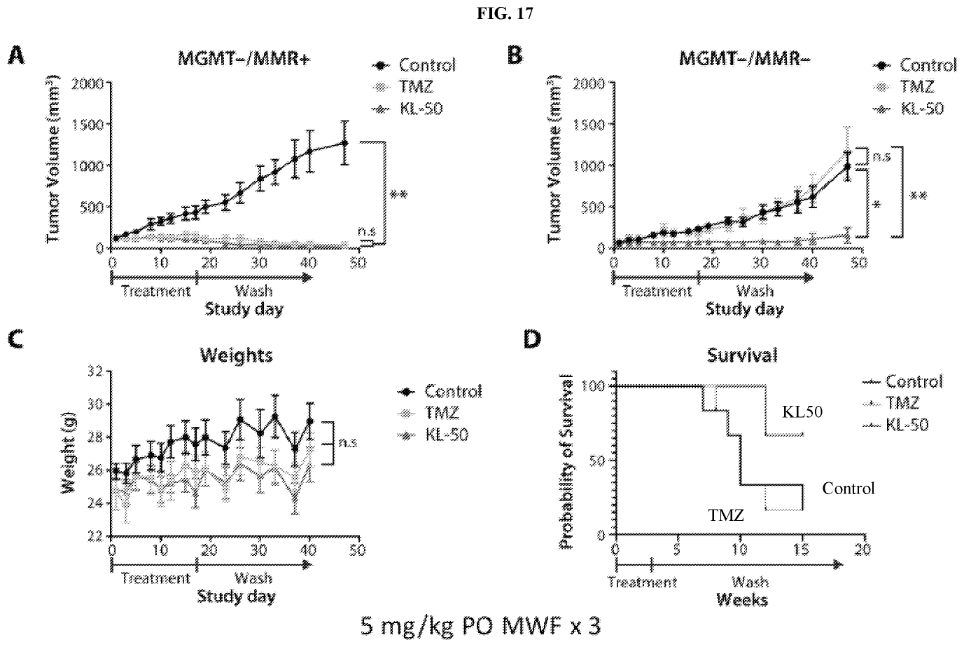

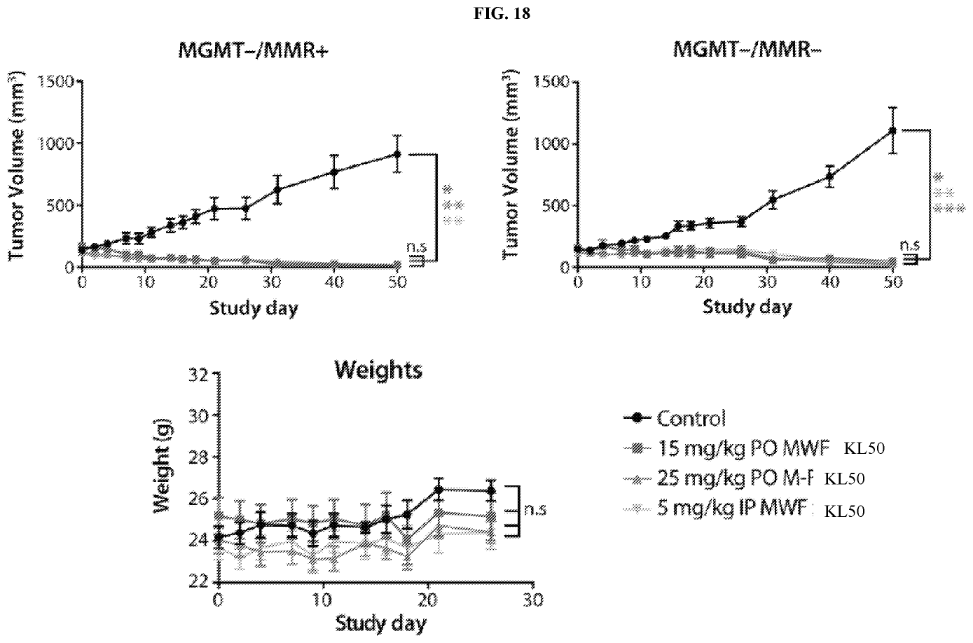

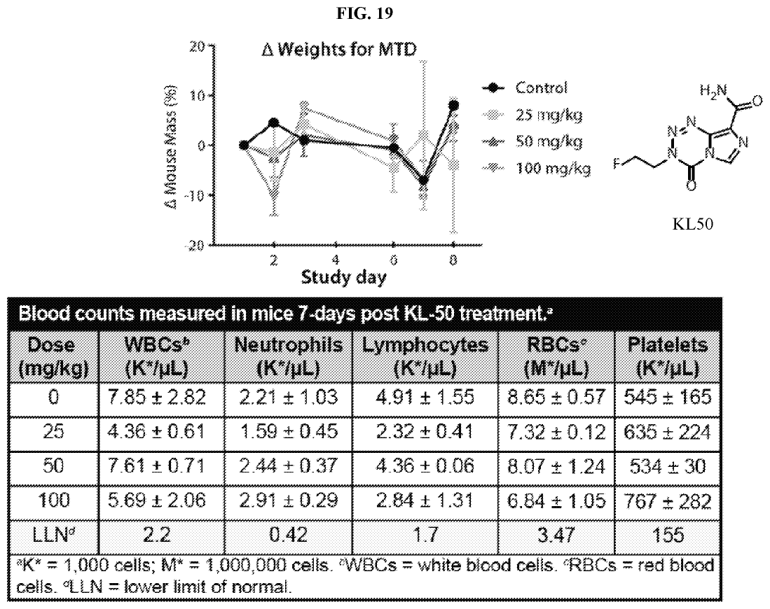

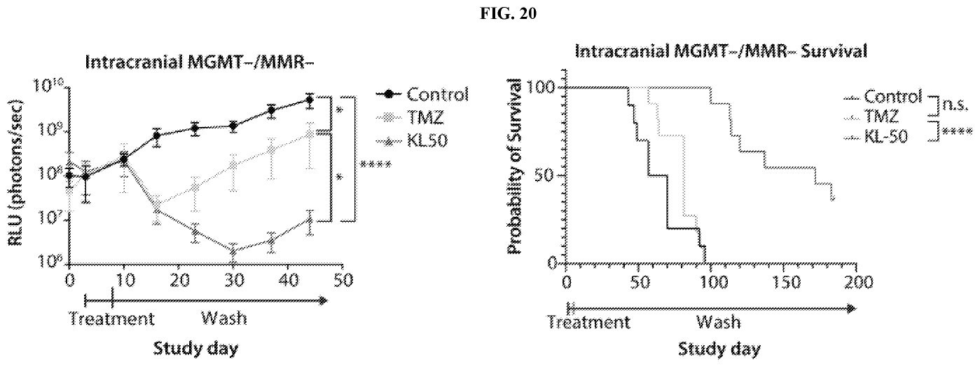

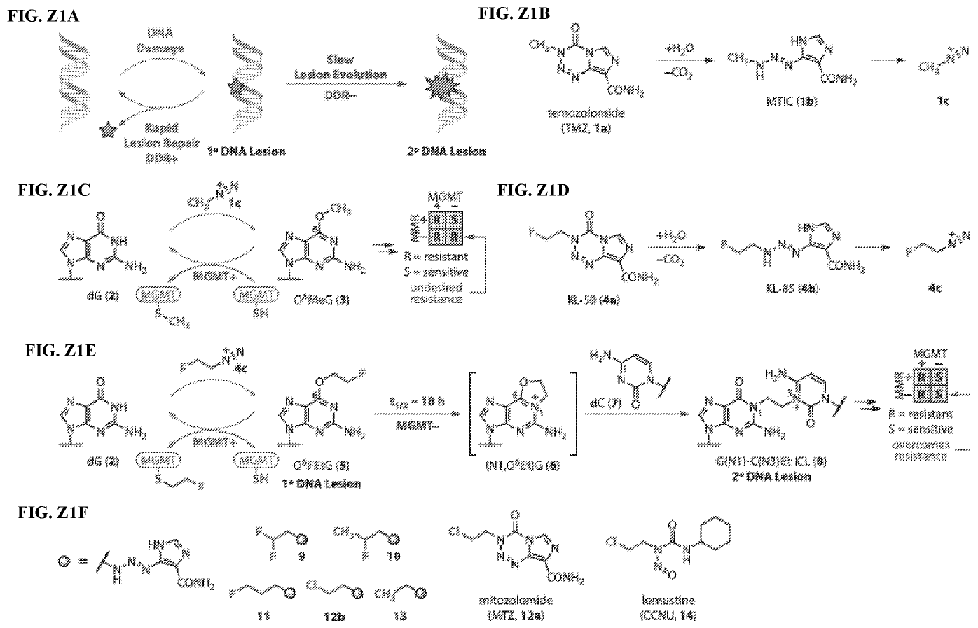

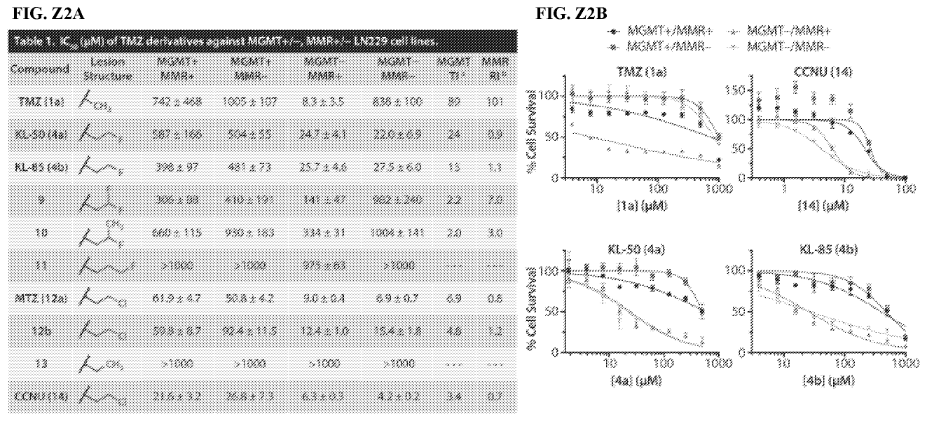

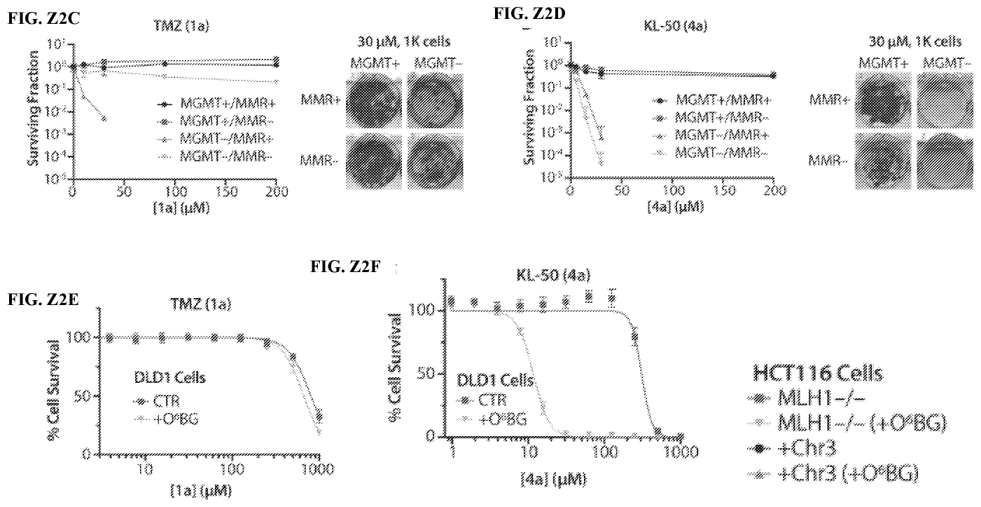

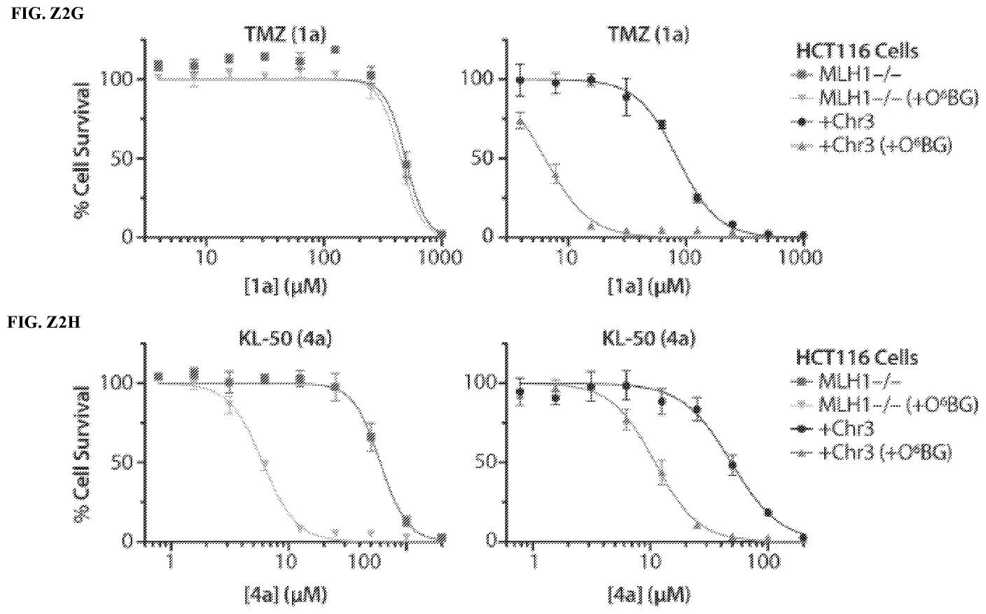

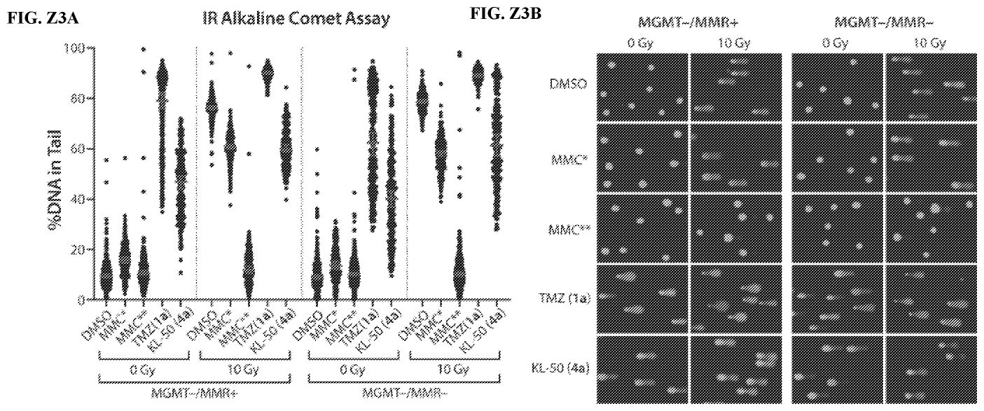

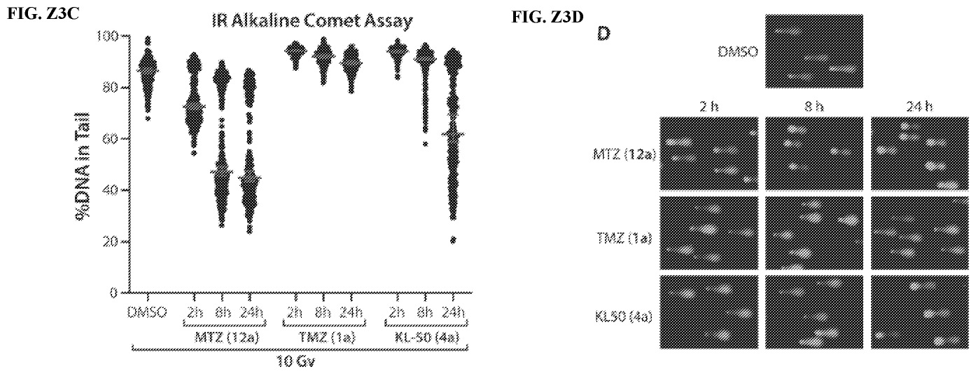

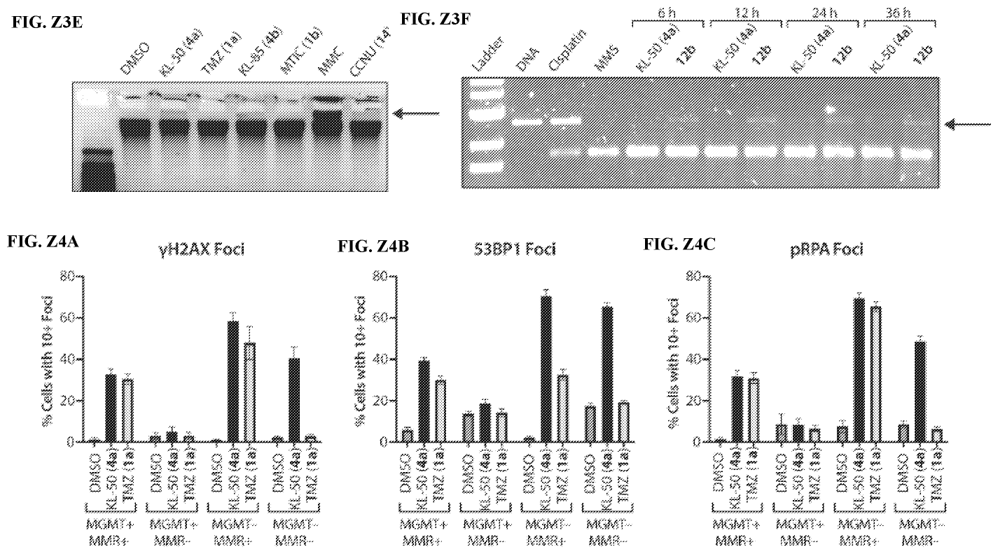

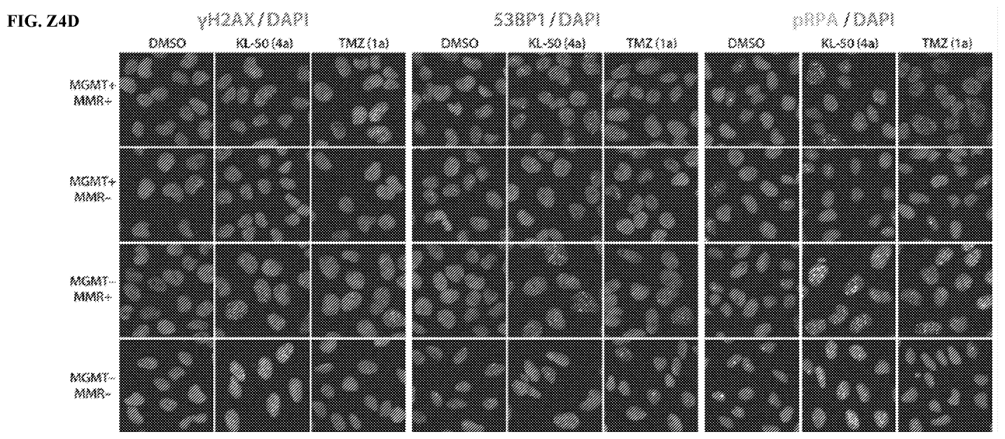

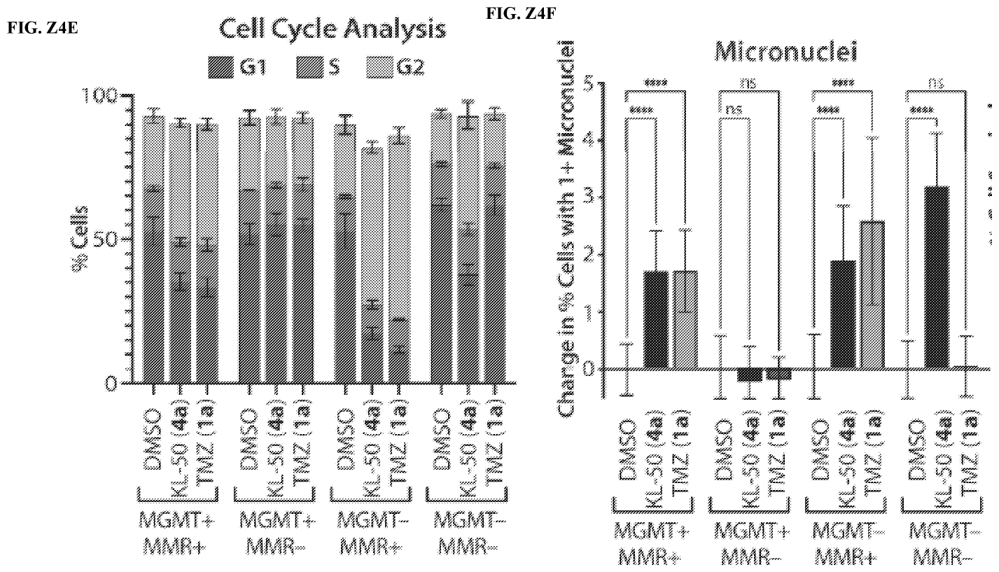

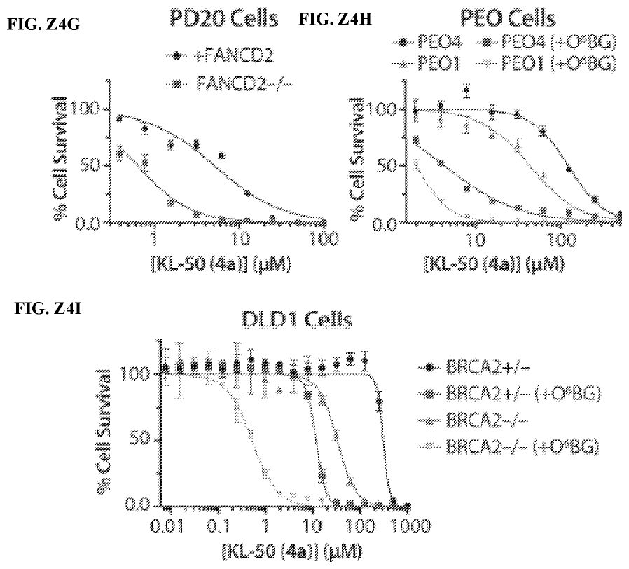

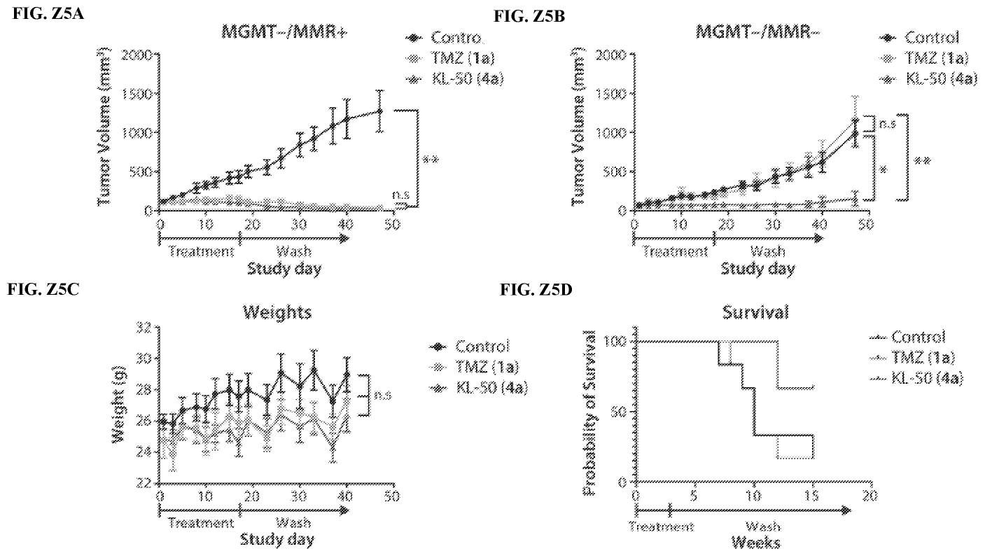

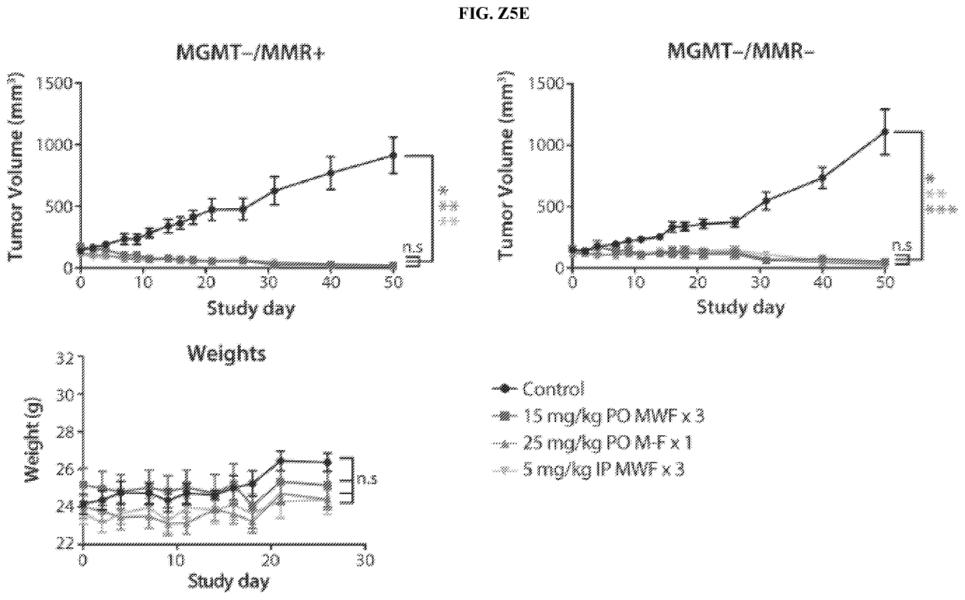

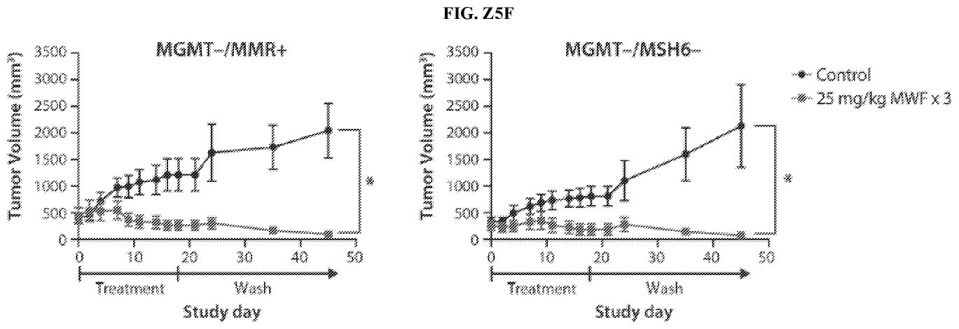

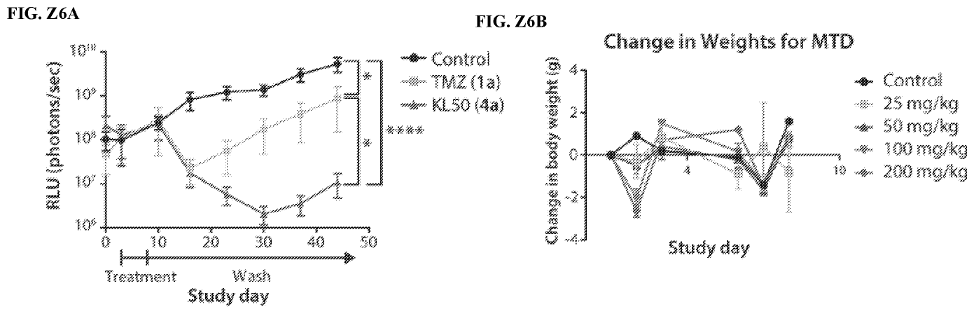

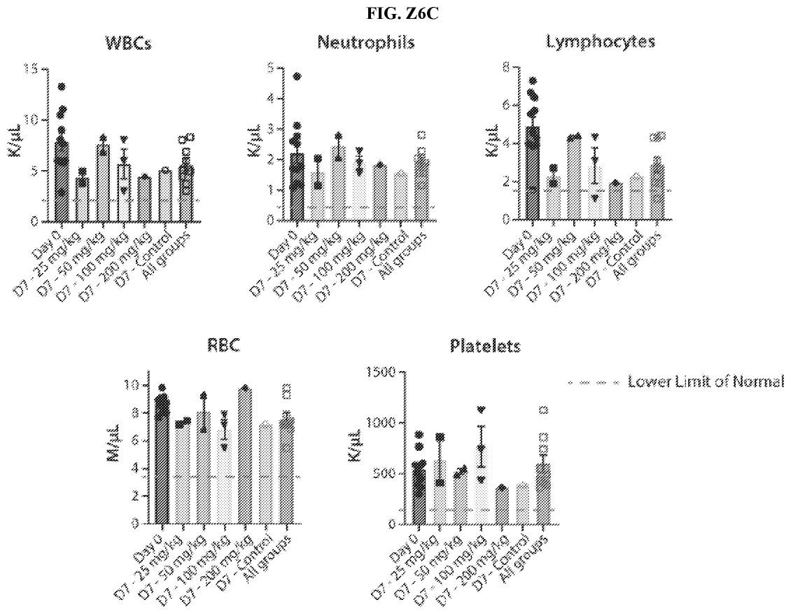

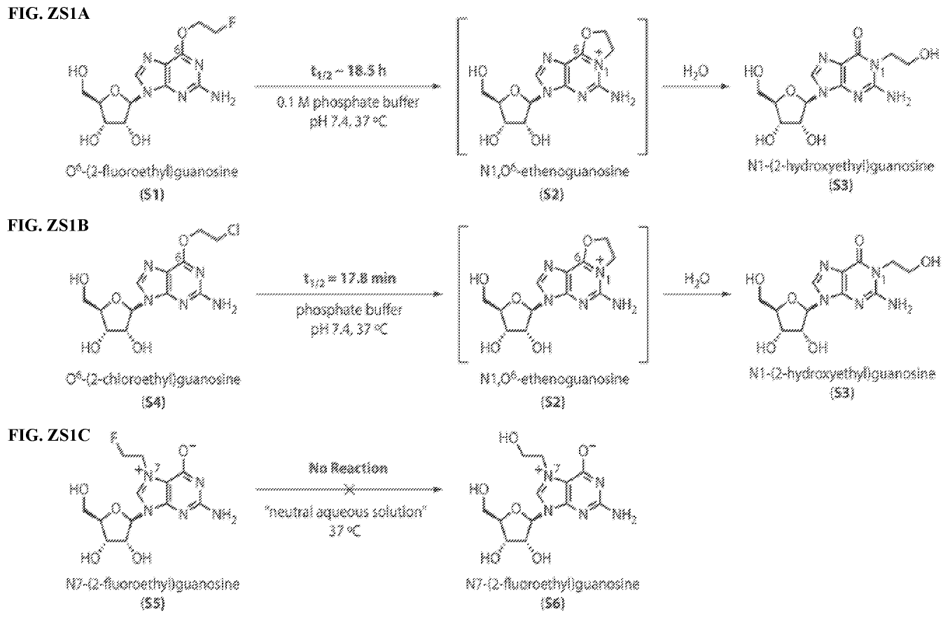

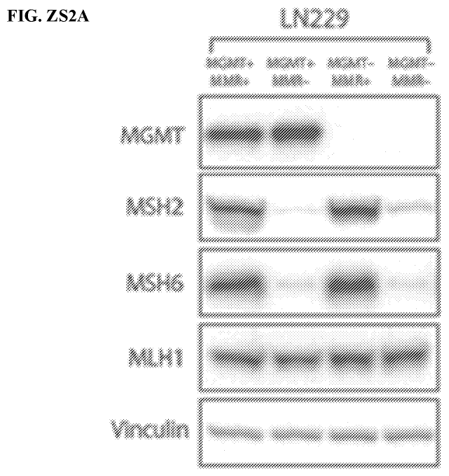

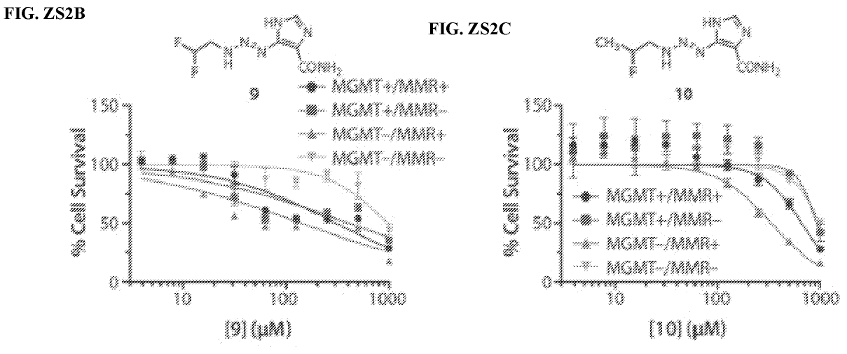

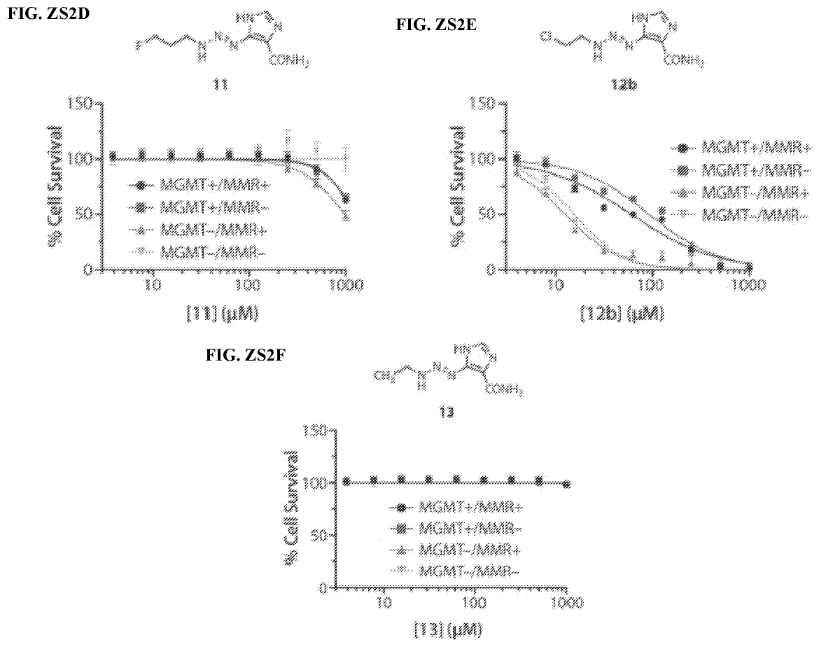

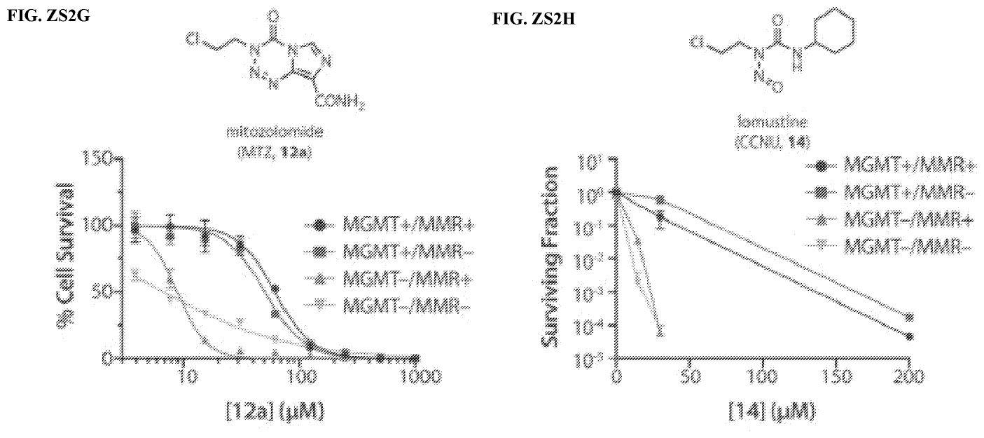

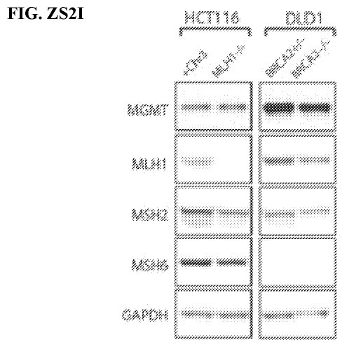

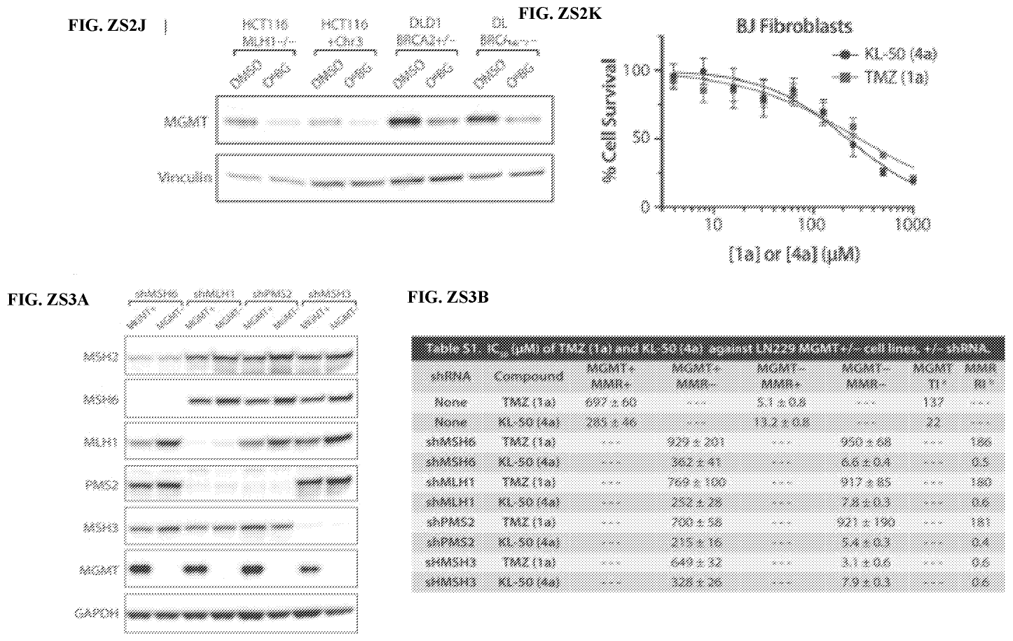

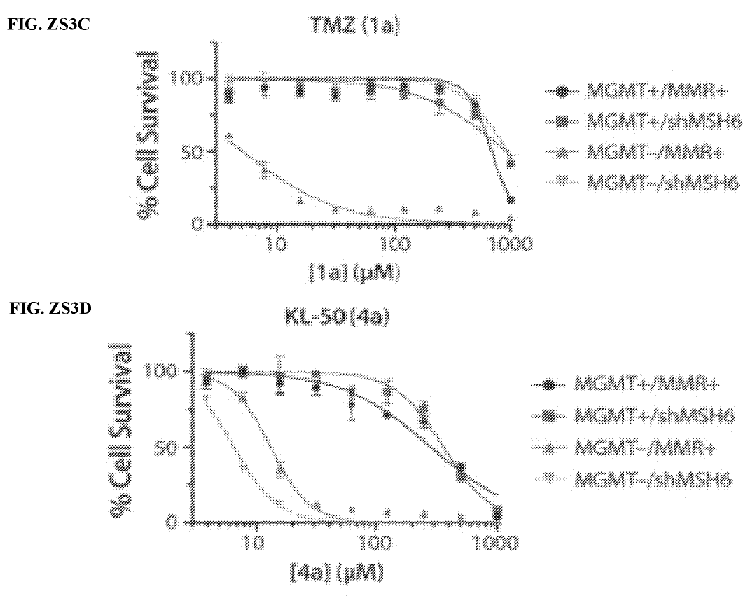

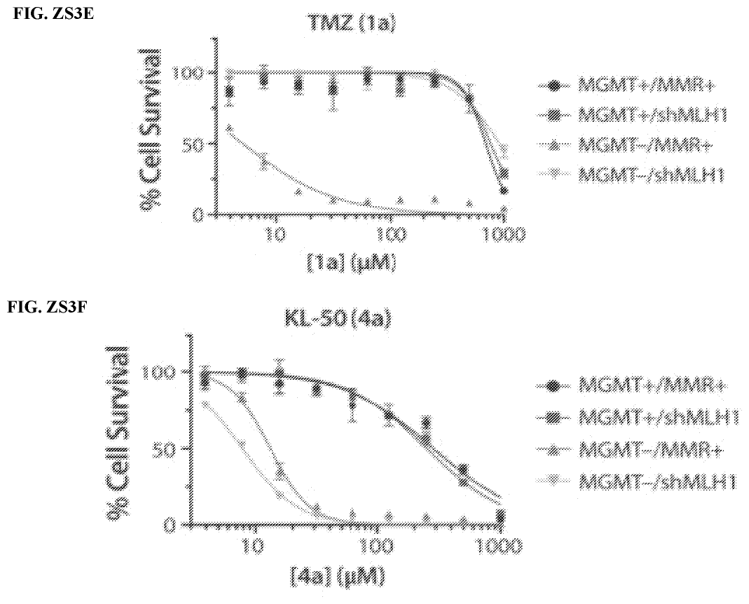

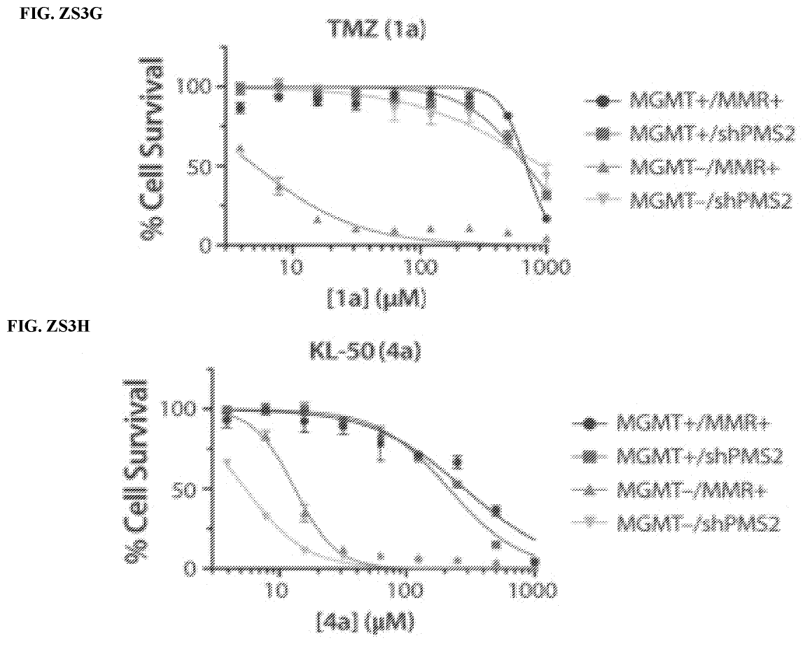

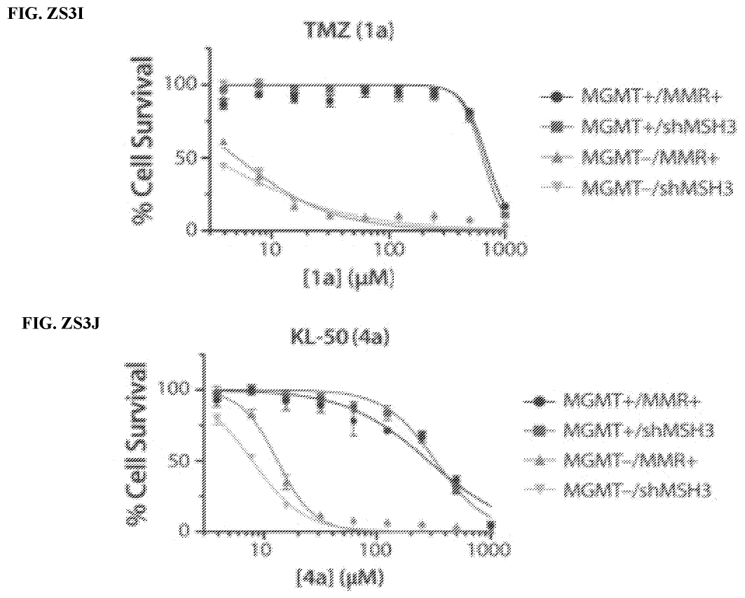

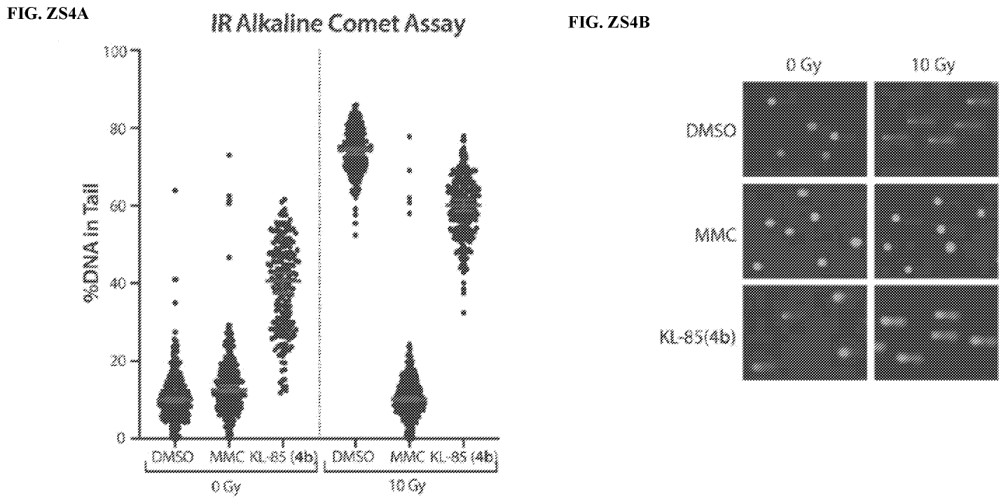

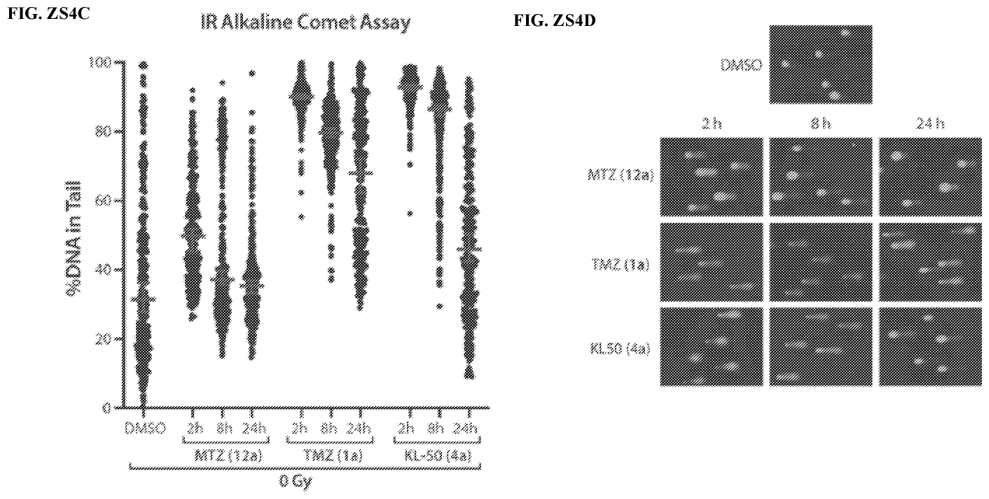

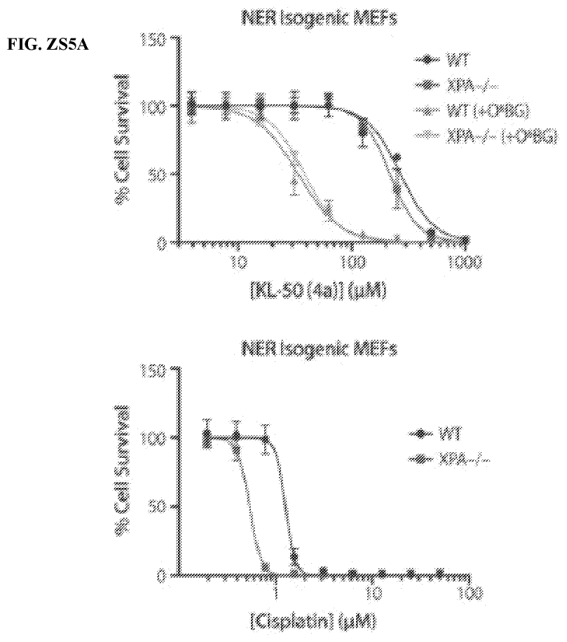

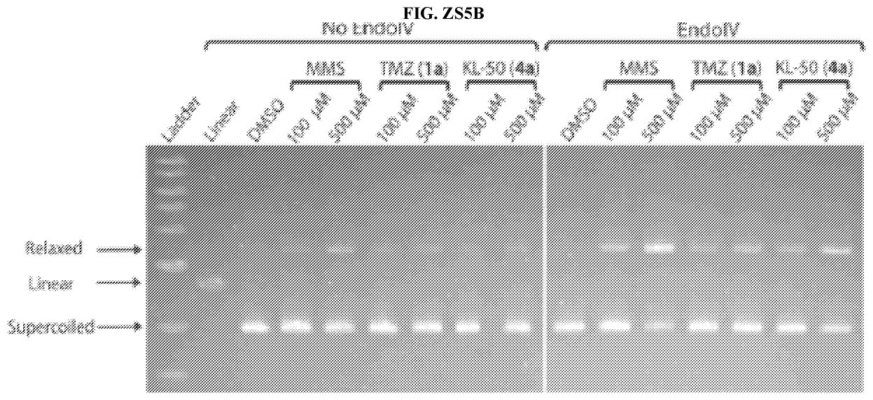

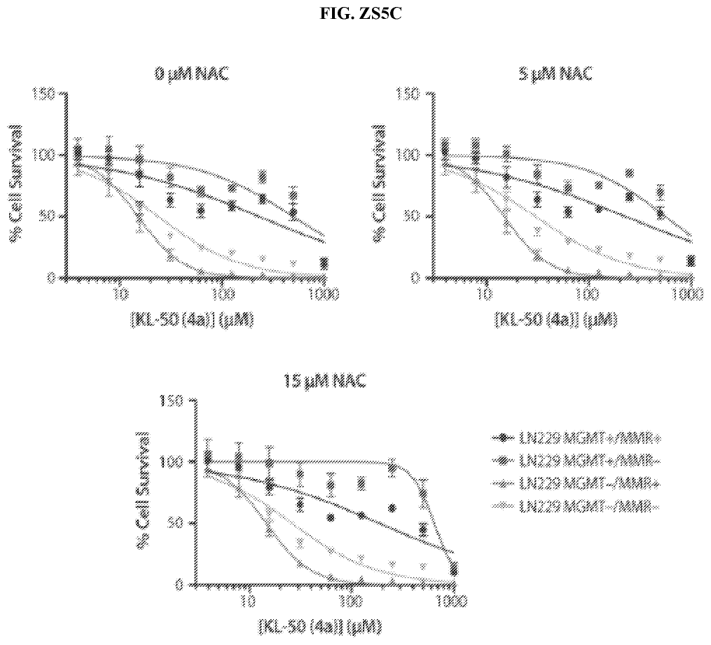

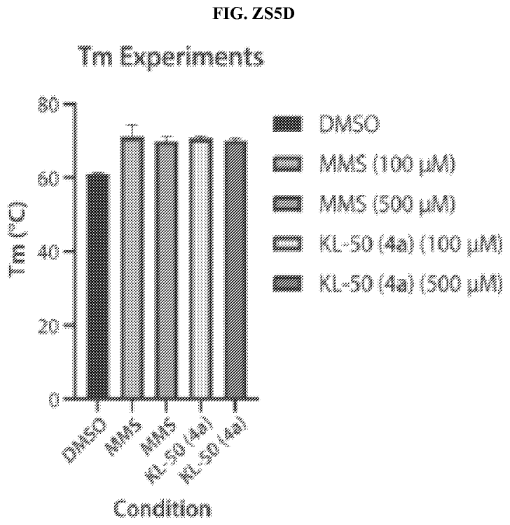

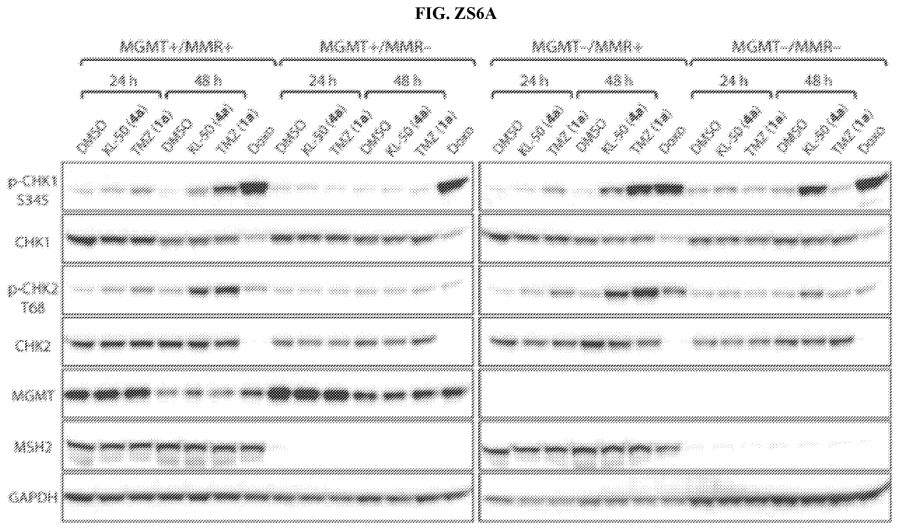

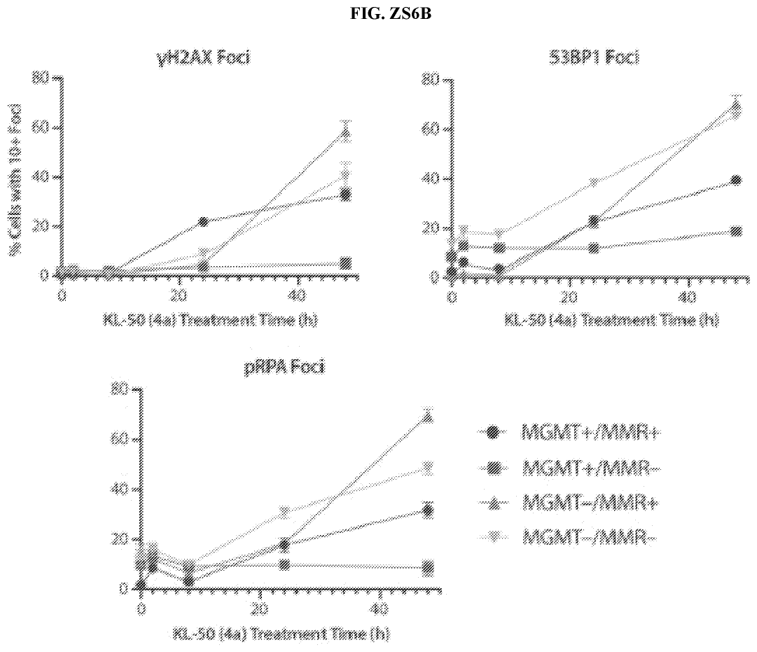

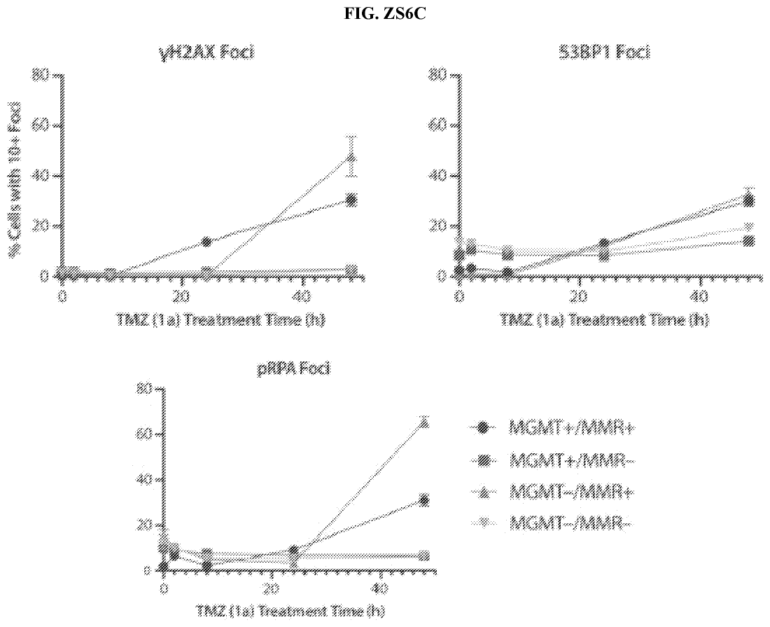

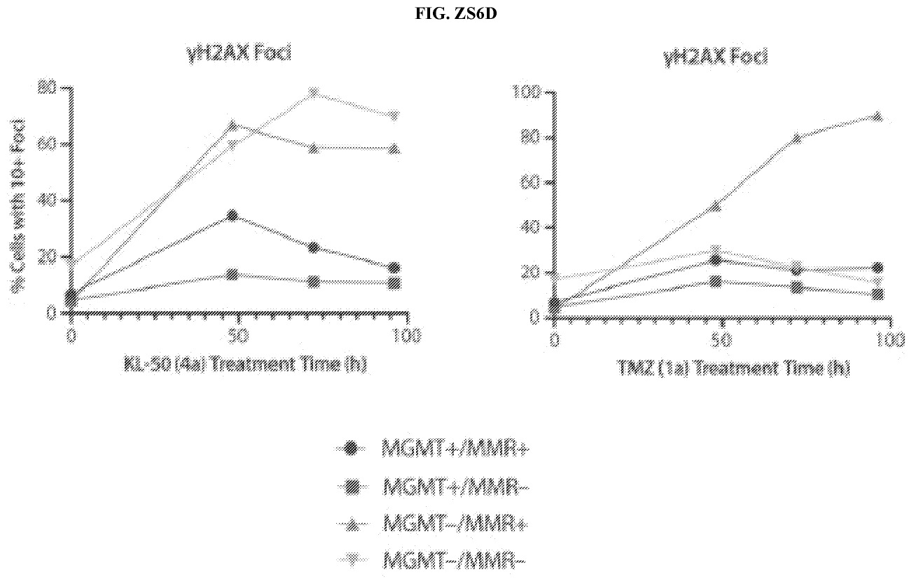

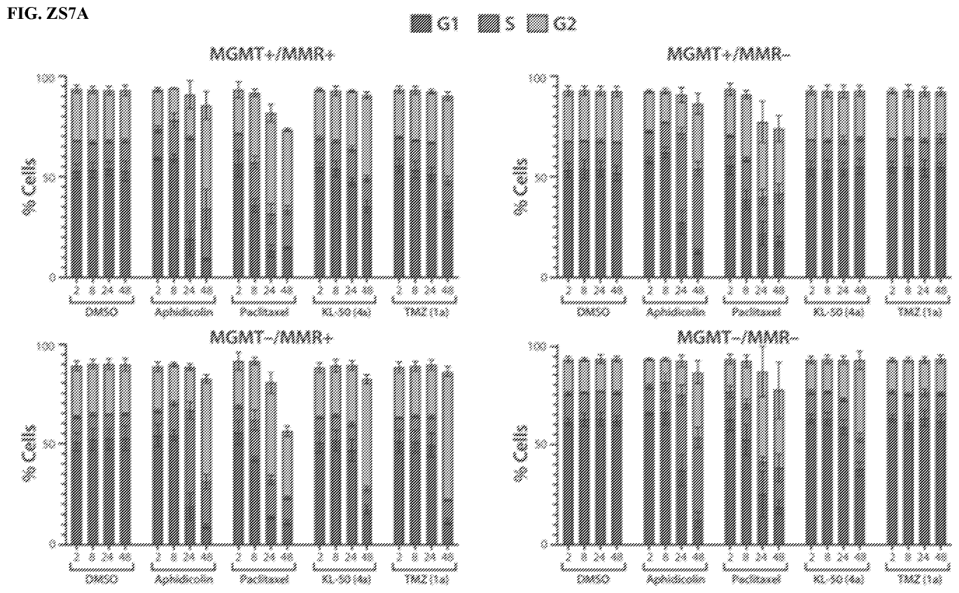

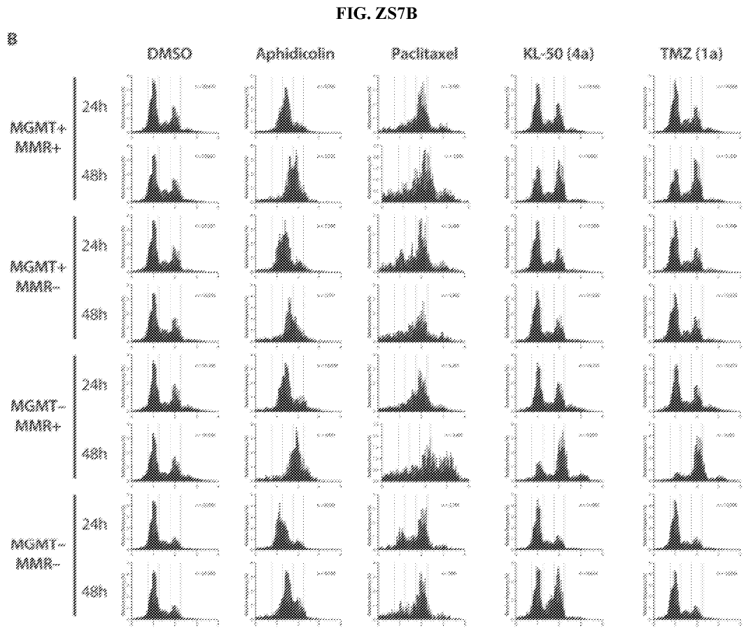

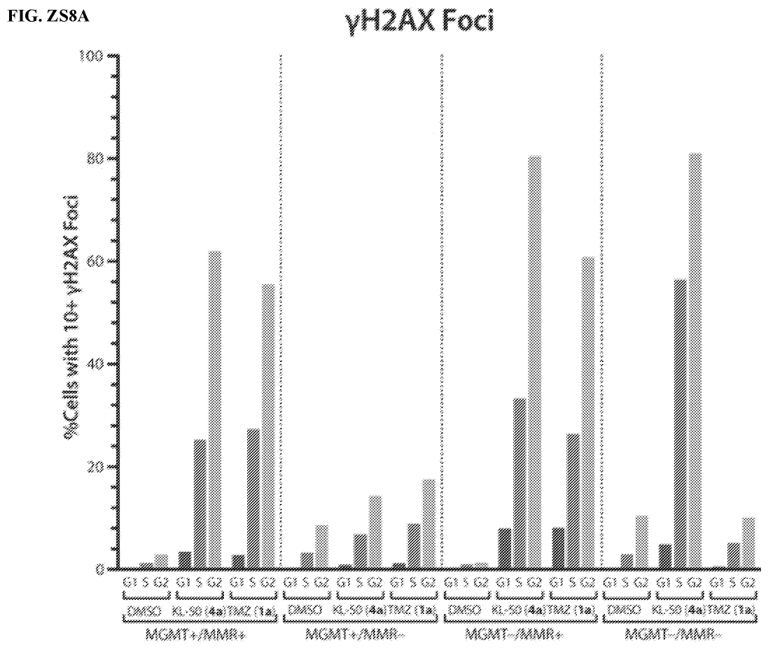

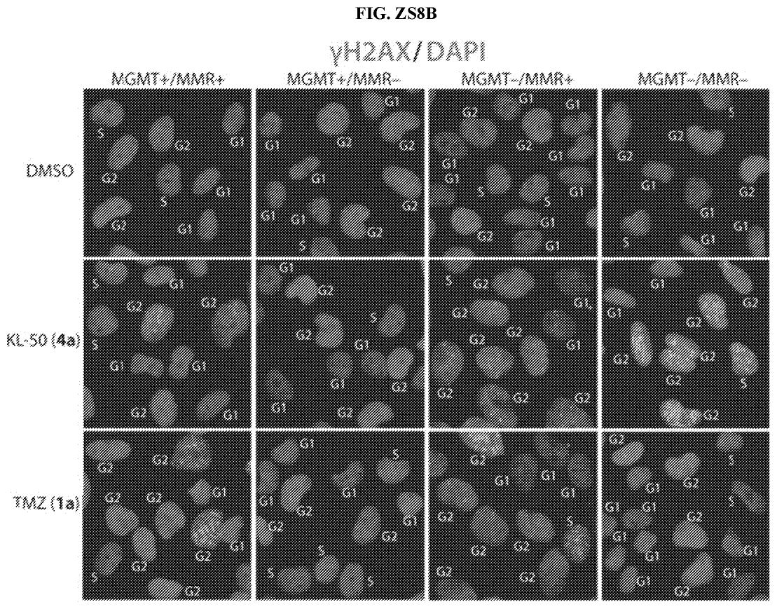

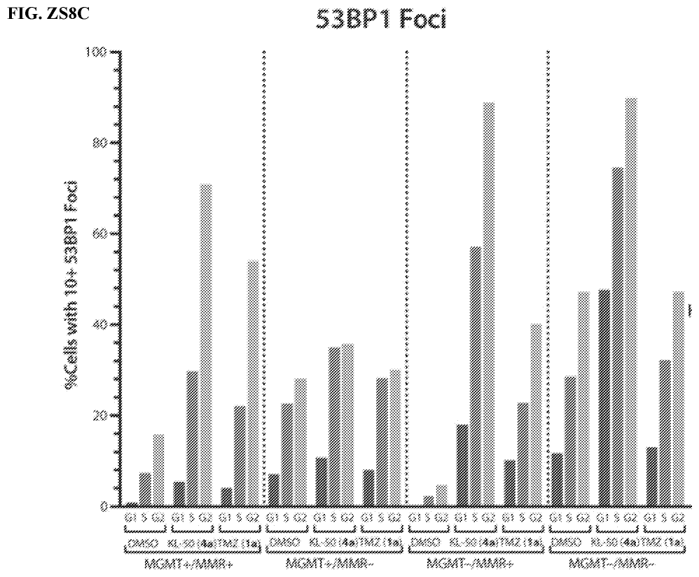

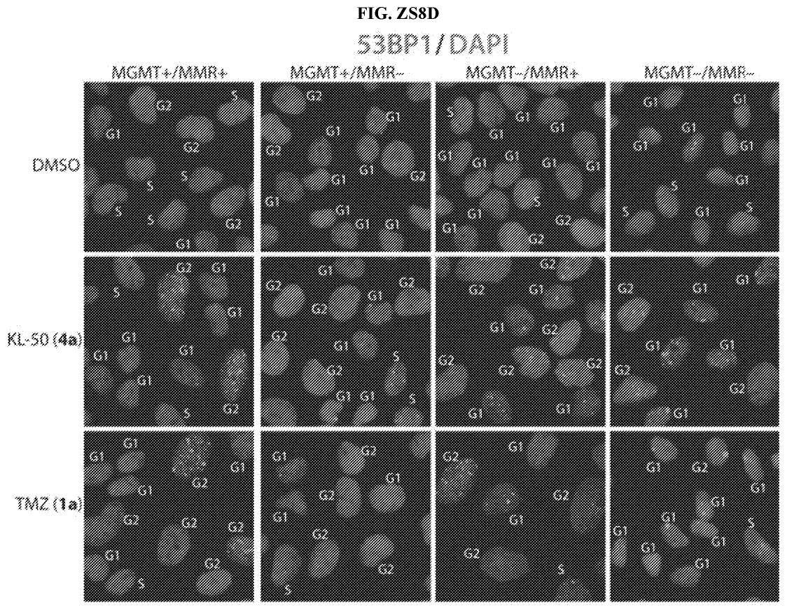

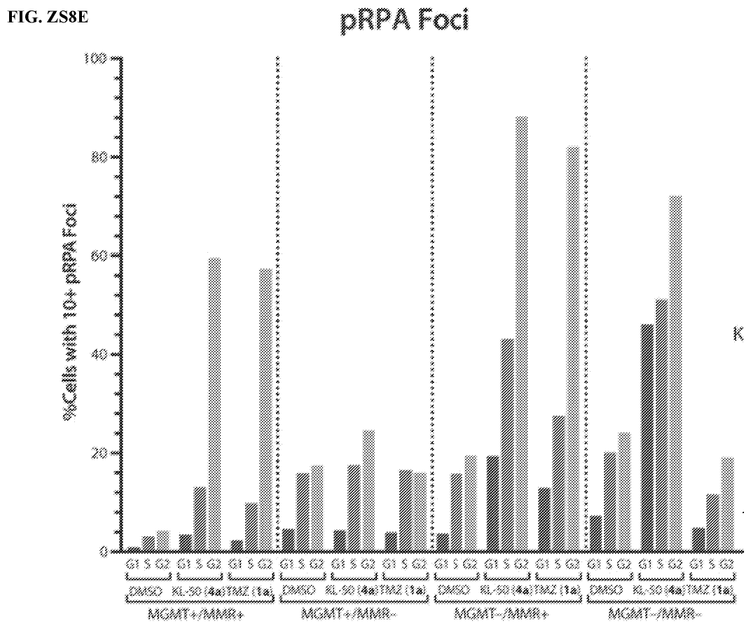

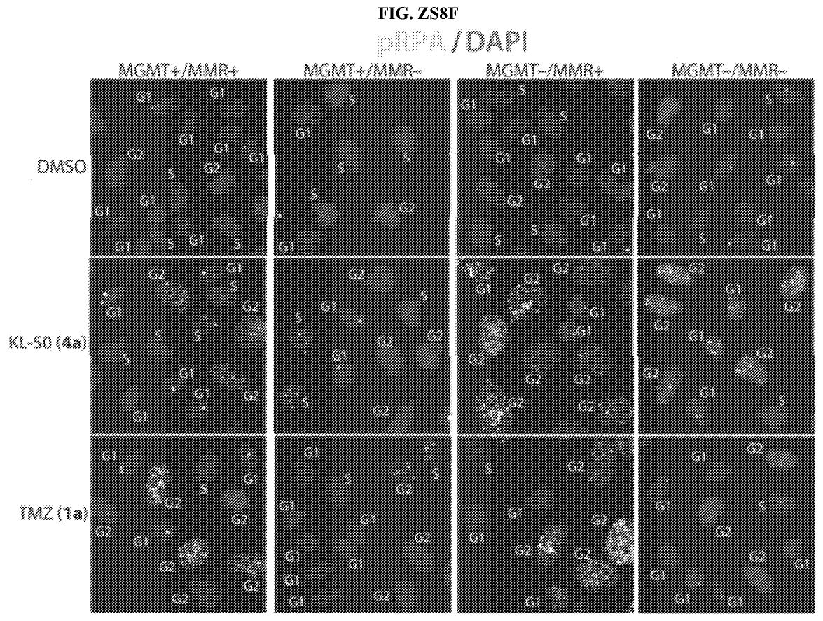

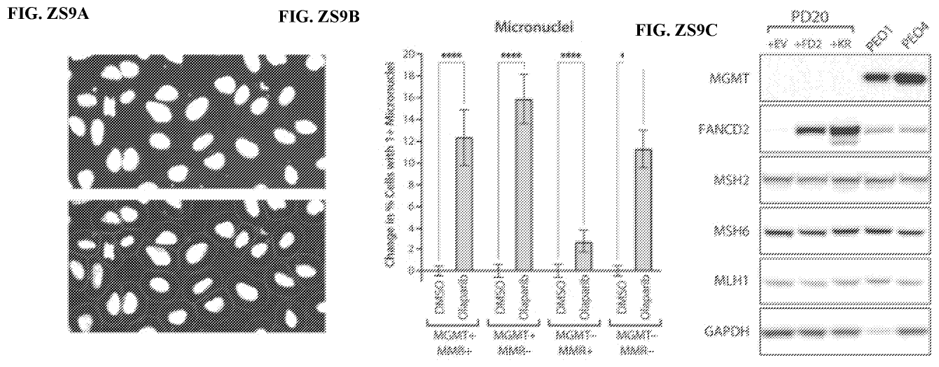

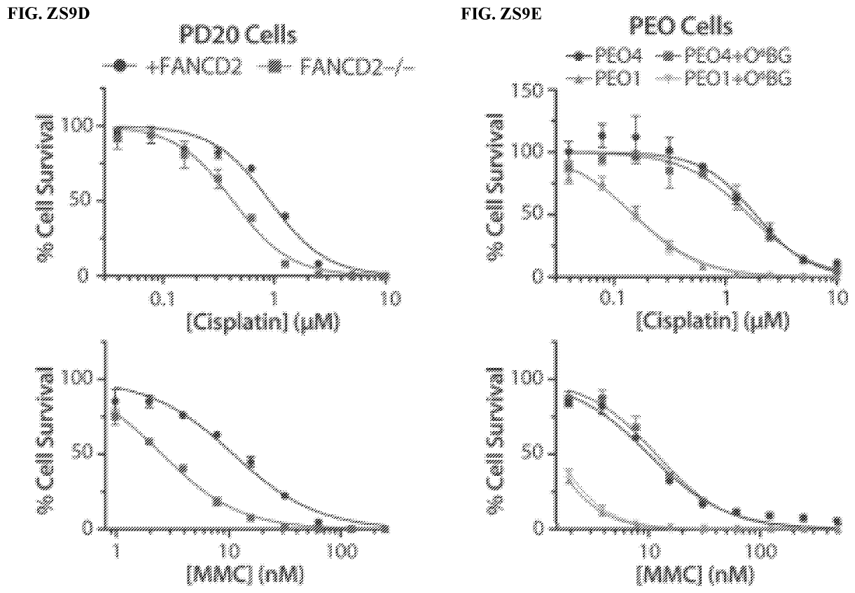

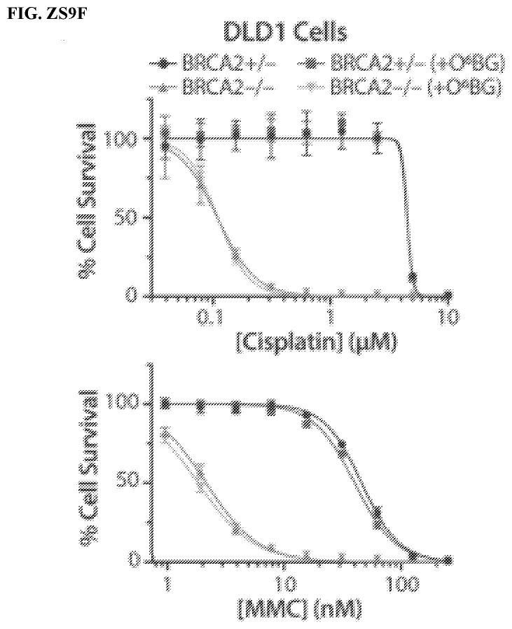

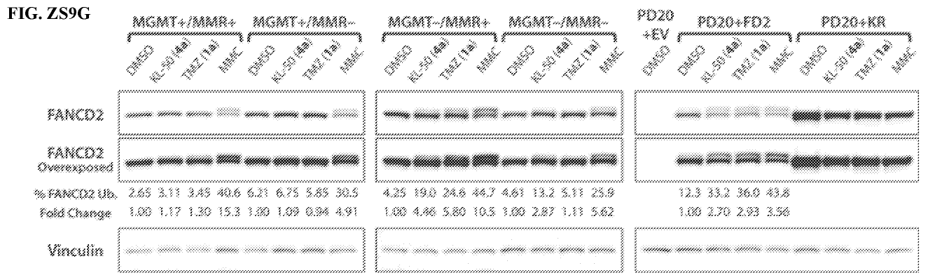

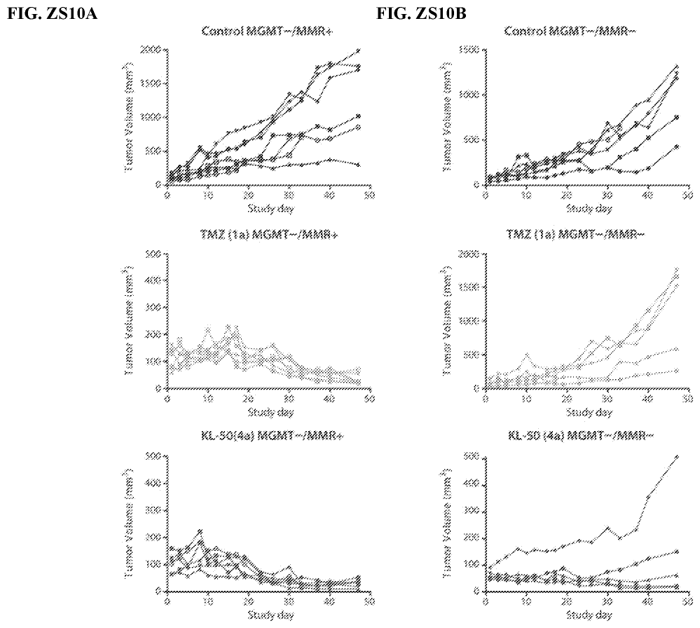

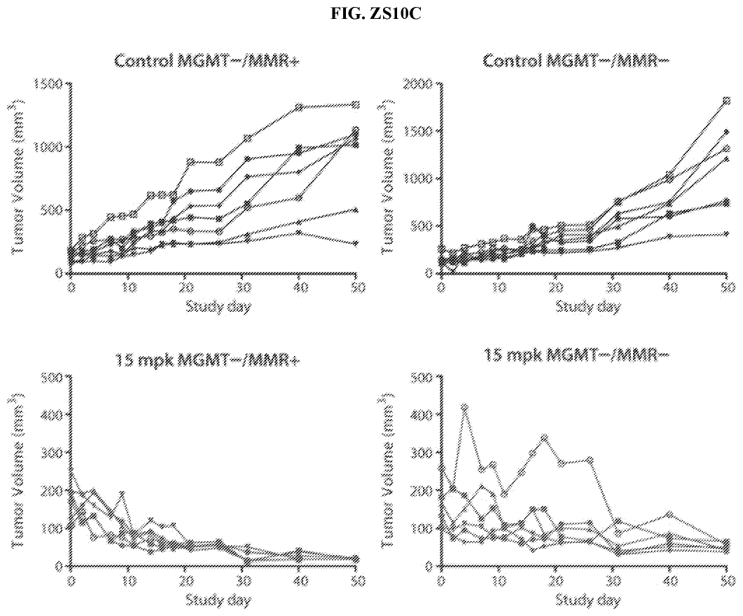

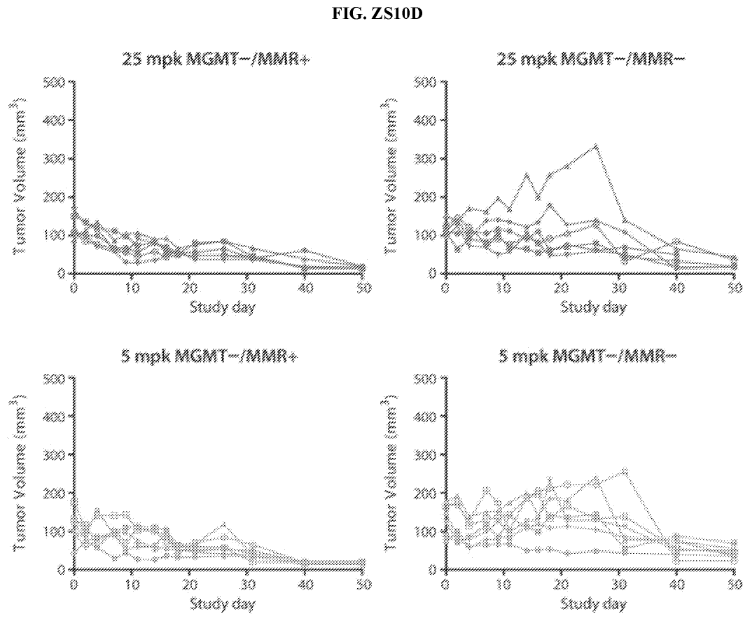

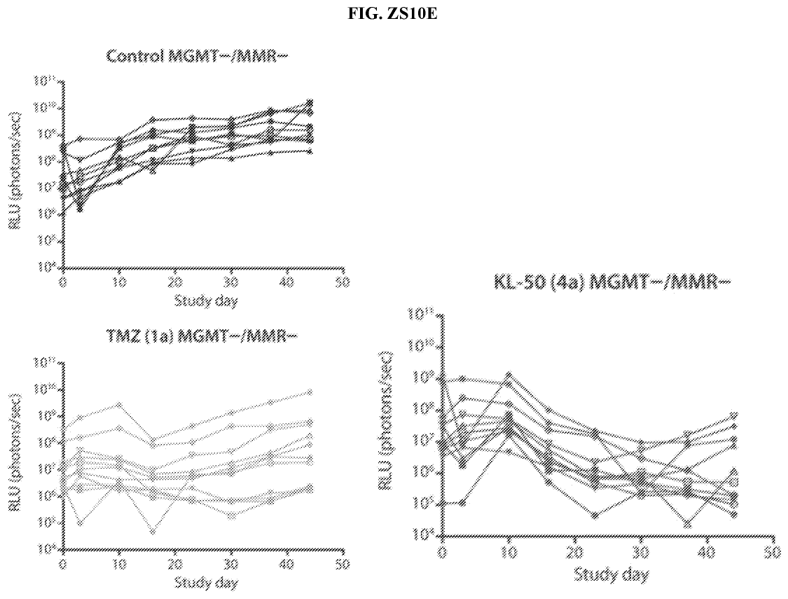

Xenograft Experiments (FIG. 11): All animal studies were approved by the Institutional Animal Use and Care Committee and performed in accordance with the Guide for the Care and Use of Laboratory Animals. LN229 WT and LN229-MSH2⁻ cell lines were maintained in DMEM media supplemented with 10% fetal bovine serum. Three-four-week-old female athymic nude Foxnnu mice were obtained from Envigo and each mouse was inoculated subcutaneously with tumor cells (4.5-5×10⁶) in 0.1 ml of PBS with Matrigel (1:1). Wild type cells were injected on the right flank and mutant cells were injected on the left flank. The tumors were then grown to a mean size of approximately 50-100 mm3 and the mice were then split into groups and treated as detailed in FIG. 7. Gavage doses of 5 mg/kg of KL-50 or TMZ were prepared by diluting stocks in DMSO with 10% cyclodextrin. Compound was administered each day of dosing at a volume of 100 μL/mouse. Mice were treated for 3 weeks with dosing on Mondays, Wednesdays and Fridays. Tumors were measured 3 times a week during treatment and during the washout period of 2 weeks. Xenograft tumors were measured by calipers and volume was calculated using the equation for ellipsoid volume: Volume=0.523×(length)×(width)². Statistical Analysis: Analysis of variance (ANOVA) was used to test for significant differences between groups. Post-hoc Bonferroni multiple comparison test analysis was used to determine significant differences among means. All statistical analysis was accomplished using Graph Pad Prism 8.2.0 software.

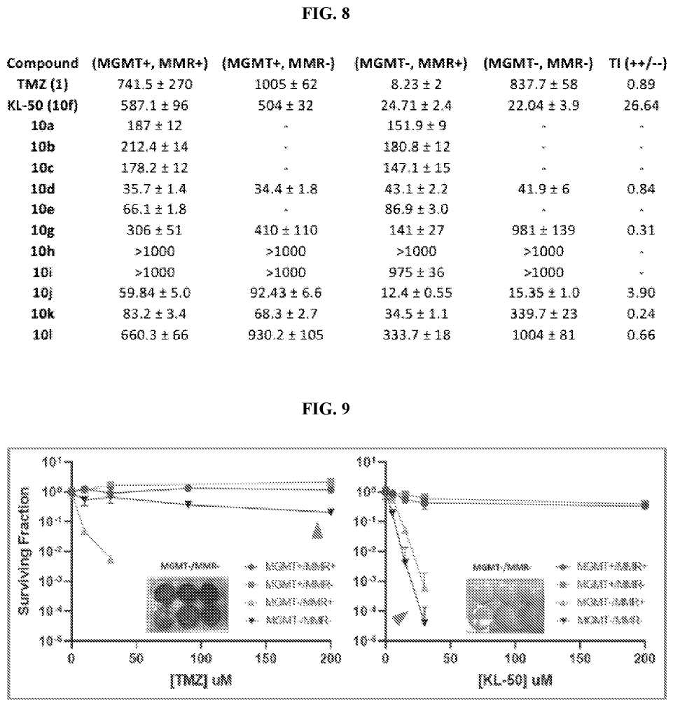

The compounds 10a through 10l and TMZ were tested in the short-term cell viability assay. The results of the testing are shown in FIG. 8.

As shown in FIG. 8, TMZ and all tested derivatives except the compounds 10f and 10j had therapeutic indices in the short-term viability assay of less than one, calculated as the IC₅₀ test results in MGMT proficient and MMR proficient cells divided by the IC₅₀ test results in MGMT deficient and MMR deficient cells. The results for these derivatives are what one of skill in the art would expect, based on the test results for TMZ. Quite unexpectedly, the therapeutic index for compound 10f (also sometimes referred to as KL50) was 26.64, indicating a strong potential for effective treatment of cancers that are both MGMT and MMR deficient while sparing MGMT proficient normal cells. This result was surprising in view of the known inability of TMZ to kill MMR deficient cells and the test results for the other derivatives.

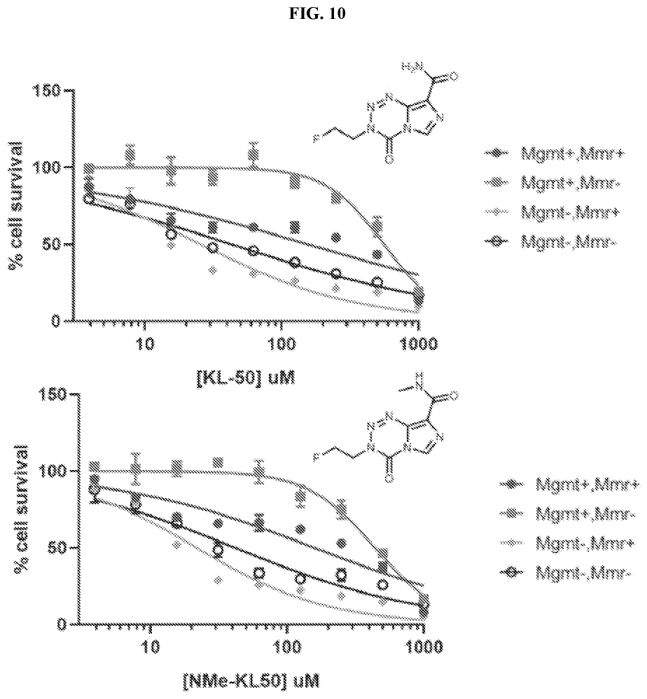

As shown in FIG. 10, KL50 and N-methyl KL50 were dose response tested in the short-term cell viability assay. Results for N-methyl KL50 were comparable to those for KL50 itself. Both compounds showed preferable toxicity for MGMT− cells compared to MGMT⁺ cells, regardless of MMR status. As for KL50, these results for N-methyl KL50 are surprising in view of the test results for TMZ and the other compounds of FIG. 3.

Compound 10f was tested in the clonogenic survival assay (compared to TMZ). As shown in FIG. 9, MGMT⁻/MMR⁻ cells were resistant to TMZ and many survived at the tested concentrations. Conversely, nearly all the MGMT−/MMR− cells failed to survive at the tested concentrations of compound 10f (KL50). That is, at the identical concentrations of test compound, about 100% of cells survived following treatment with TMZ, while less than 10⁻⁴ cells survived following treatment with KL50. This result validated the surprising results of the short-term cell viability assay.

The clonogenic survival assay is a well-recognized assay with high prediction of utility of cancer treatment compounds. See, for example, Fiebig et al.—Clonogenic assay with established human tumor xenografts: correlation of in vitro to in vivo activity as a basis for anti-cancer drug discovery—European J. Cancer, 40 (2004) 802-820. Although the short-term cell viability assay is recognized to be not as predictive of clinical usefulness as the clonogenic survival assay, negative results in the short-term cell viability assay (FIG. 4) are understood to indicate the tested compound is not a candidate for further study. Thus, the negative results for the tested compounds other than KL50 evidenced that (like TMZ) they were unable to prevent the growth of MMR deficient cells and thus were not candidates for further investigation.

Compound 10f (KL50) was compared to TMZ in the xenograph experiments described above. As can be seen from FIG. 11, KL50 has comparable activity to TMZ in MGMT⁻/MMR (MSH2)⁺ tumors (top graph) but is surprisingly effective in MGMT−/MMR (MSH2)⁻ tumors, while TMZ is ineffective. The results in this in vivo xenograft model further confirm the findings that KL50 and N-alkyl KL50 are effective cancer-treatment agents.

### Example 13: Synthesis of 3-(2-fluoroethyl)-4-oxo-3H,4H-imidazo[4,3-d][1,2,3,5]tetrazine-8-carbonyl chloride

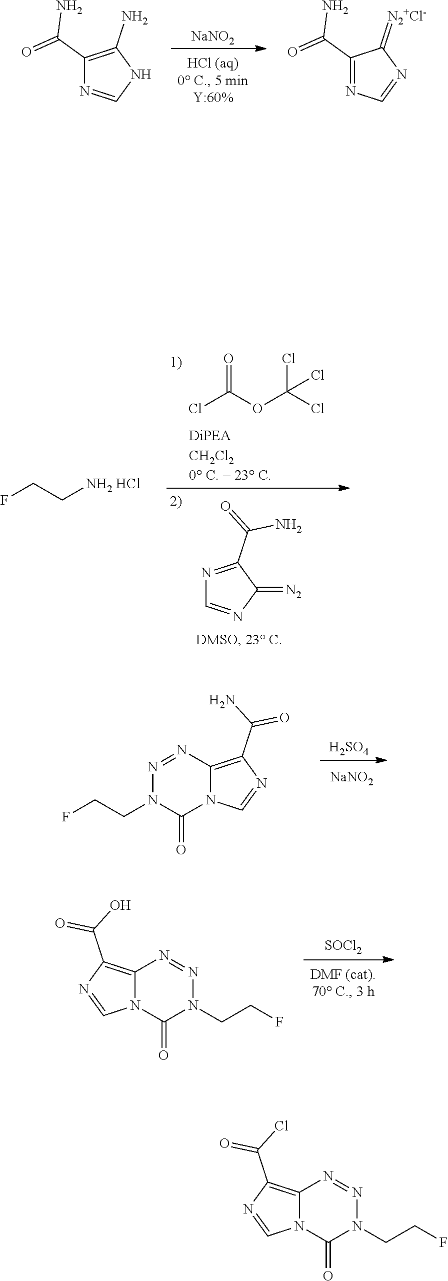

Step 1: To a stirred solution of NaNO₂ (4.74 g, 68.65 mmol) in water (78 mL) at 0° C. was added a solution of aminoimidazole hydrochloride (7.8 g, 64.85 mmol) dissolved in 1.0 M aqueous HCl (78 mL), dropwise for 10 min. The precipitate began to form after a small portion of aminoimidazole solution was added. The reaction mixture was stirred at this temperature for 5 min. The precipitate formed was filtered off, and washed with water (2×200 mL). The pale yellow puffy solid was dried under P₂O₅ for 4 h; (6 g, 56% yield). ¹H NMR (400 MHz, DMSO-d6): 7.98 (brs, 1H, CONH2), 7.78 (brs, 1H, CONH2), 7.58 (s, 1H, CH).

Step 2: A mixture of 2-fluoroethylamine hydrochloride (16.6 g, 166.78 mmol, 1 eq), and N,N-diisopropyl ethylamine (45.3 mL, 350.2 mmol, 2.1 eq) in dichloromethane (400 mL) was added dropwise via syringe pump over 45 min to a solution of diphosgene (19.7 g, 100 mmol, 0.60 eq) in dichloromethane (400 mL) at 0° C. Upon completion of the addition, the cooling bath was removed, and the reaction mixture was allowed to warm to 23° C. over 15 min. The product mixture was transferred to a separatory funnel. The organic layer was washed sequentially with 1 N aqueous hydrochloric acid solution (500 mL, precooled to 0° C.) and saturated aqueous sodium chloride solution (500 mL, precooled to 0° C.). The washed organic layer was dried over magnesium sulfate. The dried solution was filtered, and the filtrate was concentrated at 10-15° C. The unpurified isocyanate obtained (˜12 g) was used directly in the following step.

Step 3: The unpurified isocyanate (12 g) obtained in the preceding step (nominally 16.7 mmol, 1.75 eq) was added dropwise via syringe to a solution of the diazonium ion 2 (6.55 g, 47.7 mmol, 1.0 eq) in dimethyl sulfoxide (50 mL) at 23° C. Upon completion of the addition, the reaction vessel was covered with aluminum foil. The reaction mixture was stirred at 23° C. for 16 h. After completion, the reaction mixture was added dropwise into DCM (300 mL). The mixture was stirred for 1 h and the solid was filtrated, washed with DCM (50 mL) and concentrated to 3-(2-fluoroethyl)-4-oxo-3H,4H-imidazo[4,3-d][1,2,3,5]tetrazine-8-carboxamide as a pink crystalline powder (9 g, 83% based on the diazonium ion 2). ¹H NMR (400 MHz, DMSO-d6): δ 8.83 (s, 1H), 7.83 (s, 1H), 7.70 (s, 1H), 4.87-4.55 (m, 4H).

Step 4: 3-(2-fluoroethyl)-4-oxo-3H,4H-imidazo[4,3-d][1,2,3,5]tetrazine-8-carboxamide (27.8 g, 123 mmol, 1.0 eq) was dissolved in sulfuric acid (192 mL) and cooled to 0° C. in an ice bath. Separately, NaNO₂ (31 g, 448.6 mmol, 3.65 eq) was dissolved in 125 mL of water (precooled to 0° C.). The solution of NaNO₂ was added dropwise slowly to the ice-cold sulfuric acid solution and the reaction was allowed to gradually warm to room temperature overnight. The reaction was cooled to 0° C., 560 mL of ice water was added, and the reaction was stirred for 1 h. Then solids were collected by vacuum filtration. Solids were dried under vacuum to afford 3-(2-fluoroethyl)-4-oxo-3H,4H-imidazo[4,3-d][1,2,3,5]tetrazine-8-carboxylic acid (22 g, 78%) as off-white solid. LC-MS: 228.0 [M+1]+. ¹H NMR (400 MHz, DMSO-d6): δ 8.82 (s, 1H), 5.17-4.26 (m, 4H).

Step 5: 3-(2-fluoroethyl)-4-oxo-3H,4H-imidazo[4,3-d][1,2,3,5]tetrazine-8-carboxylic acid (1.5 g, 0.006 mol, 1.0 equiv.) was dissolved in SOCl₂ (33.6 mL, 0.042 mol, 70 equiv.) at room temperature. DMF (0.01 mL, 0.05 equiv.) was added to the reaction at room temperature. The reaction mixture was stirred for 3 hours at 80° C. After, the reaction was cooled to room temperature and concentrated to dryness under reduced pressure to afford the titled compound as a crude yellow solid (1.4 g) and used in the next step without further purification.

### Example 14: Synthesis of Compound I-2: N-ethyl-3-(2-fluoroethyl)-4-oxo-3H,4H-imidazo[4,3-d][1,2,3,5]tetrazine-8-carboxamide

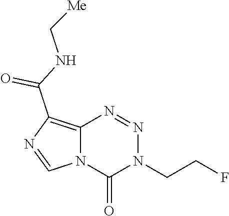 [SMILES: CCNC(=O)c1ncn2c(=O)n(CCF)nnc12]

A flask was charged with ethylamine (18 mg, 4.07 mmol, 1.0 equiv.) in THF (5 volumes) and triethylamine (3.0 equiv.), followed by a solution of 3-(2-fluoroethyl)-4-oxo-3H,4H-imidazo[4,3-d][1,2,3,5]tetrazine-8-carbonyl chloride (100 mg, 4.07 mmol, 1.0 equiv.) (Example 13) in THF (5 volumes) at 0° C. The reaction mixture was stirred for 16 h at room temperature. After the reaction was complete, the reaction mixture was concentrated, and the residue was purified by flash chromatography to provide the title compound as a brown solid (22 mg, 19%) ¹H NMR (400 MHz, DMSO-d6) δ=8.90-8.80 (m, 1H), 8.52 (br d, J=4.9 Hz, 1H), 4.92-4.85 (m, 1H), 4.80-4.74 (m, 1H), 4.69-4.63 (m, 1H), 4.61-4.56 (m, 1H), 3.35-3.32 (m, 2H), 1.16-1.11 (m, 3H); LCMS: 98.55%; ESI MS m/z calcd. For C₉H₁₁FN₆O₂ ([M+H]+) 255.1; found 255.3; HPLC: 99.13%.

### Example 15: Preparation of 3-(2-fluoroethyl)-N-methyl-4-oxo-3H,4H-imidazo[4,3-d][1,2,3,5]tetrazine-8-carboxamide (I-1)

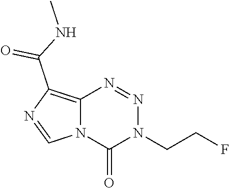 [SMILES: CNC(=O)c1ncn2c(=O)n(CCF)nnc12]

To a round bottom flask containing 3-(2-fluoroethyl)-4-oxo-3,4-dihydroimidazo[5,1-d][1,2,3,5]tetrazine-8-carbonyl chloride (1.4 g, 0.0057 mol, 1.0 equiv.) was added THF (14 mL, 10 V) at room temperature. Next, a solution of methanamine hydrochloride (766 mg, 0.011 mol, 2.0 eq.) and N,N-diisopropylethylamine (1.54 g, 0.012 mol, 2.1 eq) in THF (14 mL, 10 V) was added into the reaction mixture over 10 minutes. The reaction was stirred for 5 h at room temperature. Then, ethyl acetate (50 mL) and brine (50 mL) were added to the reaction mixture. The resulting aqueous layer was separated and extracted with ethyl acetate (3×50 mL). The organic phase was collected and concentrated to dryness by rotary evaporation to provide a residue. The residue was purified by reversed-phase flash chromatography with the following conditions: column, AQ C18 silica gel; mobile phase, Water (0.05% Formic acid) in ACN, 0% to 20% gradient in 30 min; detector, UV 254 nm to afford 3-(2-fluoroethyl)-N-methyl-4-oxo-3,4-dihydroimidazo[5,1-d][1,2,3,5]tetrazine-8-carboxamide as an off-white solid. (900 mg, 65%) 1H NMR (400 MHz, DMSO-d6) δ 8.88 (s, 1H), 8.56-8.41 (m, 1H), 4.89 (dd, J=5.2, 4.3 Hz, 1H), 4.77 (dd, J=5.2, 4.3 Hz, 1H), 4.69-4.63 (m, 1H), 4.62-4.56 (m, 1H), 2.82 (d, J=4.8 Hz, 3H); ESI MS m/z calcd. For C8H9FN6O2 ([M+H]+) 241.20; found 241.10.

### Example 16: Synthesis of Additional Amide Compounds

The following general procedure was to prepare additional amide compounds. A flask was charged with amine (1.0 equiv.) in THF (5 volumes) and triethylamine (3.0 equiv.), followed by a solution of 3-(2-fluoroethyl)-4-oxo-3H,4H-imidazo[4,3-d][1,2,3,5]tetrazine-8-carbonyl chloride (1.0 equiv.) in THF (5 volumes) at 0° C. The reaction mixture was stirred for 16 h at room temperature. After the reaction was complete, the reaction mixture was concentrated and the residue was purified by flash chromatography to provide the titled compound.

Compounds prepared according to the above general procedure include the following:

### Compound I-3: 3-(2-fluoroethyl)-4-oxo-N-(2,2,2-trifluoroethyl)-3H,4H-imidazo[4,3-d][1,2,3,5]tetrazine-carboxamide

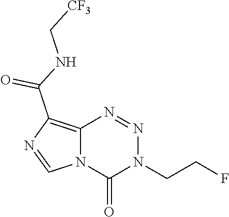 [SMILES: O=C(NCC(F)(F)F)c1ncn2c(=O)n(CCF)nnc12]

The title compound was prepared using the general procedure on an 80 mg scale to provide the title compound as a white solid (48 mg, 44%). ¹H NMR (400 MHz, DMSO-d6) δ=9.07 (t, J=6.4 Hz, 1H), 8.92 (s, 1H), 4.89 (t, J=4.8 Hz, 1H), 4.78 (t, J=4.8 Hz, 1H), 4.68 (t, J=4.7 Hz, 1H), 4.61 (t, J=4.7 Hz, 1H), 4.16-4.03 (m, 2H); LCMS: 97.27%; ESI MS m/z calcd. For C₉H₈F₄N₆O₂ [M+H]+ 309.0; found 309.2; HPLC: 98.12%

### Compound I-4: N-cyclopropyl-3-(2-fluoroethyl)-4-oxo-3H,4H-imidazo[4,3-d][1,2,3,5]tetrazine-8-carboxamide

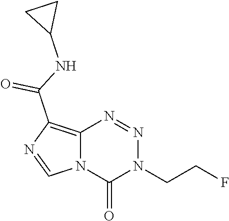 [SMILES: O=C(NC1CC1)c1ncn2c(=O)n(CCF)nnc12]

The title compound was prepared using the general procedure on 100 mg scale to provide the title compound as a brown solid (37 mg, 32%) ¹H NMR (400 MHz, DMSO-d6) δ=8.85 (s, 1H), 8.51 (d, J=4.5 Hz, 1H), 4.88 (t, J=4.8 Hz, 1H), 4.77 (t, J=4.8 Hz, 1H), 4.65 (t, J=4.8 Hz, 1H), 4.59 (t, J=4.8 Hz, 1H), 2.95-2.85 (m, 1H), 0.74-0.62 (m, 4H); LCMS: 99.67%; ESI MS m/z calcd. For C₁₀H₁₁FN₆O₂ ([M+H]+) 268.1; found 268.2; HPLC: 98.14%

### Compound I-5: N-cyclobutyl-3-(2-fluoroethyl)-4-oxo-3H,4H-imidazo[4,3-d][1,2,3,5]tetrazine-8-carboxamide

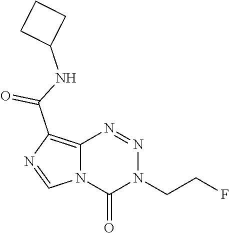 [SMILES: O=C(NC1CCC1)c1ncn2c(=O)n(CCF)nnc12]

The title compound was prepared using the general procedure on 80 mg scale to provide the title compound as a pale brown solid (22 mg, 22%) ¹H NMR (400 MHz, DMSO-d6) δ=8.87 (s, 1H), 8.67 (d, J=8.1 Hz, 1H), 4.88 (t, J=4.6 Hz, 1H), 4.76 (t, J=4.6 Hz, 1H), 4.65 (t, J=4.6 Hz, 1H), 4.59 (t, J=4.6 Hz, 1H), 4.47 (qd, J=8.4, 16.6 Hz, 1H), 2.27-2.10 (m, 4H), 1.72-1.59 (m, 2H); LCMS: 99.06%; ESI MS m/z calcd. For C₁₁H₁₃FN₆O₂ ([M+H]+) 282.1; found 281.2; HPLC: 99.34%

### Compound I-6: 3-(2-fluoroethyl)-N,N-dimethyl-4-oxo-3H,4H-imidazo[4,3-d][1,2,3,5]tetrazine-8-carboxamide

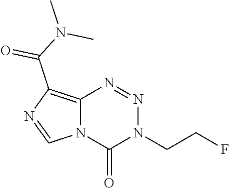 [SMILES: CN(C)C(=O)c1ncn2c(=O)n(CCF)nnc12]

The title compound was prepared using the general procedure on 80 mg scale to provide the titled compound as a brown solid (8.6 mg, 9%) ¹H NMR (400 MHz, DMSO-d6) δ=8.85 (s, 1H), 4.88 (t, J=4.8 Hz, 1H), 4.76 (m, 1H), 4.63 (t, J=4.8 Hz, 1H), 4.57 (t, J=4.8 Hz, 1H), 3.06 (s, 3H), 3.05 (s, 3H); LCMS: 99.74%; ESI MS m/z calcd. For C₉H₁₁FN₆O₂ [M+H]+ 255.1; found 255.2; HPLC: 99.70%

### Compound I-7: 3-(2-fluoroethyl)-4-oxo-N-(1,3-thiazol-2-yl)-3H,4H-imidazo[4,3-d][1,2,3,5]tetrazine-8-carboxamide

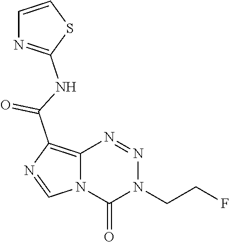 [SMILES: O=C(Nc1nccs1)c1ncn2c(=O)n(CCF)nnc12]

The title compound was prepared using the general procedure on 100 mg scale to provide the title compound as a pale yellow solid (22 mg, 16%) ¹H NMR (400 MHz, DMSO-d6) δ=12.22 (br s, 1H), 8.99 (s, 1H), 7.57 (d, J=2.8 Hz, 1H), 7.33 (d, J=3.0 Hz, 1H), 4.91 (br s, 1H), 4.79 (br s, 1H), 4.70 (br s, 1H), 4.63 (br s, 1H); LCMS: 99.81%; ESI MS m/z calcd. For C₁₀H₈FN₇O₂S ([M+H]+) 309.2; found 310.1; HPLC: 99.74%.

### Compound I-8: 3-(2-fluoroethyl)-4-oxo-N-(pyridin-2-yl)-3H,4H-imidazo[4,3-d][1,2,3,5]tetrazine-8-carboxamide

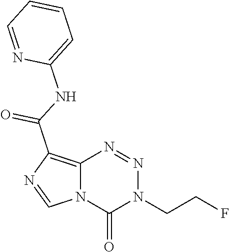 [SMILES: O=C(Nc1ccccn1)c1ncn2c(=O)n(CCF)nnc12]

The title compound was prepared using the general procedure on 200 mg scale to provide the title compound as a white solid (40 mg, 15%) ¹H NMR (400 MHz, DMSO-d6) δ=9.97 (s, 1H), 9.00 (s, 1H), 8.40 (d, J=4.6 Hz, 1H), 8.25 (d, J=8.3 Hz, 1H), 7.91 (t, J=8.2 Hz, 1H), 7.26-7.15 (m, 1H), 4.91 (t, J=4.6 Hz, 1H), 4.79 (t, J=4.6 Hz, 1H), 4.70 (t, J=4.5 Hz, 1H), 4.64 (t, J=4.6 Hz, 1H); LCMS: 95.62%; ESI MS m/z calcd. For C₁₂H₁₀FN₇O₂ [M+H]+ 304.0; found 304.2; HPLC: 96.09%

### Compound I-9: 8-(azetidine-1-carbonyl)-3-(2-fluoroethyl)-3H,4H-imidazo[4,3-d][1,2,3,5]tetrazin-4-one

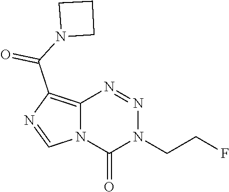 [SMILES: O=C(c1ncn2c(=O)n(CCF)nnc12)N1CCC1]

The title compound was prepared using the general procedure on 100 mg scale to provide the title compound as an off-white solid (22 mg, 18%) ¹H NMR (400 MHz, DMSO-d6) δ=8.84 (s, 1H), 4.88 (t, J=4.8 Hz, 1H), 4.76 (t, J=4.8 Hz, 1H), 4.65 (t, J=4.8 Hz, 1H), 4.59 (t, J=4.8 Hz, 1H), 4.48 (t, J=7.7 Hz, 2H), 4.10 (t, J=7.8 Hz, 2H), 2.31 (quin, J=7.8 Hz, 2H); LCMS: 99.06%; ESI MS m/z calcd. For C₁₁H₁₃FN₆O₂ [M+H]+ 281.2; found 281.2; HPLC: 99.34%.

### Compound I-10: 3-(2-fluoroethyl)-8-(pyrrolidine-1-carbonyl)-3H,4H-imidazo[4,3-d][1,2,3,5]tetrazin-4-one

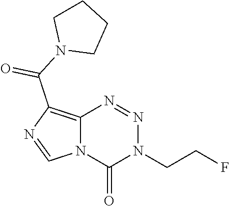 [SMILES: O=C(c1ncn2c(=O)n(CCF)nnc12)N1CCCC1]

The title compound was prepared using the general procedure on 50 mg scale to provide the title compound as a brown solid (23 mg, 37%) ¹H NMR (400 MHz, DMSO-d6) δ=8.84 (s, 1H), 4.88 (t, J=4.8 Hz, 1H), 4.76 (t, J=4.8 Hz, 1Hs), 4.64 (t, J=4.8 Hz, 1H), 4.57 (t, J=4.8 Hz, 1H), 3.63 (t, J=6.6 Hz, 2H), 3.54 (t, J=6.8 Hz, 2H), 1.92-1.84 (m, 4H); LCMS: 99.14%; ESI MS m/z calcd. For C₁₁H₁₃FN₆O₂ ([M+H]+) 281.1; found 281.2; HPLC: 97.61%.

### Compound I-11: 3-(2-fluoroethyl)-8-(4-methylpiperazine-1-carbonyl)-3H,4H-imidazo[4,3-d][1,2,3,5]tetrazin-4-one

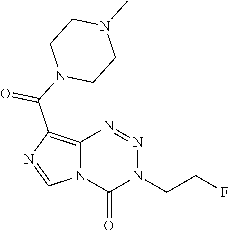 [SMILES: CN1CCN(C(=O)c2ncn3c(=O)n(CCF)nnc23)CC1]

The title compound was prepared using the general procedure on 100 mg scale to provide the title compound as a brown solid (25 mg, 18%) ¹H NMR (400 MHz, DMSO-d6) δ=8.85 (s, 1H), 4.88 (t, J=4.8 Hz, 1H), 4.76 (t, J=4.8 Hz, 1H), 4.63 (t, J=4.8 Hz, 1H), 4.57 (t, J=4.8 Hz, 1H), 3.69 (br s, 2H), 3.58-3.51 (m, 2H), 2.40 (br s, 2H), 2.35-2.28 (m, 2H), 2.21 (s, 3H); LCMS: 99.26%; ESI MS m/z calcd. For C₁₂H₁₆FN₇O₂ ([M+H]+) 310.14; found 310.2; HPLC: 95.60%

### Compound I-12: N-[2-(dimethylamino)ethyl]-3-(2-fluoroethyl)-4-oxo-3H,4H-imidazo[4,3-d][1,2,3,5]tetrazine-8-carboxamide hydrochloride

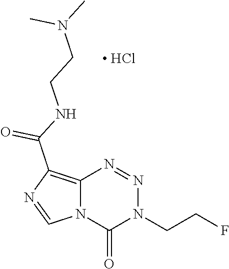 [SMILES: CN(C)CCNC(=O)c1ncn2c(=O)n(CCF)nnc12 ; "· HCl"]

The title compound was prepared using the general procedure on 100 mg scale to provide the title compound as a brown solid (45 mg, 34%) ¹H NMR (400 MHz, DMSO-d6) δ=8.93 (s, 1H), 8.85-8.76 (m, 1H), 4.89 (t, J=4.6 Hz, 1H), 4.78 (t, J=4.6 Hz, 1H), 4.67 (t, J=4.6 Hz, 1H), 4.60 (t, J=4.6 Hz, 1H), 3.67 (q, J=6.1 Hz, 2H), 3.26 (t, J=6.0 Hz, 2H), 2.81 (s, 6H); LCMS: 93.76%; ESI MS m/z calcd. For C₁₁H₁₆FN₇O₂ ([M+H]+) 298.1; found 298.2; HPLC: 98.97%.

### Compound I-13: 3-(2-fluoroethyl)-4-oxo-N,N-dipropyl-3H,4H-imidazo[4,3-d][1,2,3,5]tetrazine-8-carboxamide

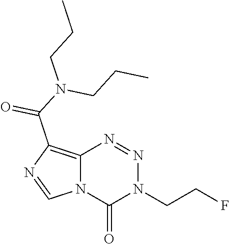 [SMILES: CCCN(CCC)C(=O)c1ncn2c(=O)n(CCF)nnc12]

The title compound was prepared using the general procedure on 100 mg scale to provide the title compound as a brown solid (90 mg, 66%) ¹H NMR (400 MHz, DMSO-d6) δ 8.83 (s, 1H) 4.74-4.91 (m, 2H) 4.52-4.65 (m, 2H) 3.43 (br t, J=7.44 Hz, 2H) 3.33 (br s, 1H) 3.30 (br s, 1H) 1.50-1.67 (m, 4H) 0.92 (t, J=7.38 Hz, 3H) 0.71 (t, J=7.38 Hz, 3H); LCMS: 99.56%; ESI MS m/z calcd. For C₁₃H₁₉FN₆O₂ ([M+H]+) 311.1; found 311.2; HPLC: 98.24%.

### Compound I-14: N-ethyl-3-(2-fluoroethyl)-N-methyl-4-oxo-3H,4H-imidazo[4,3-d][1,2,3,5]tetrazine-8-carboxamide

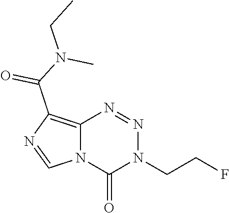 [SMILES: CCN(C)C(=O)c1ncn2c(=O)n(CCF)nnc12]

The title compound was prepared using the general procedure on 100 mg scale to provide the title compound as a brown solid (80 mg, 67%) ¹H NMR (400 MHz, DMSO-d6) δ 8.84 (s, 1H) 4.88 (t, J=4.89 Hz, 1H) 4.73-4.79 (m, 1H) 4.63 (t, J=4.65 Hz, 1H) 4.54-4.59 (m, 1H) 3.52 (q, J=7.34 Hz, 1H) 3.41 (q, J=7.01 Hz, 1H) 3.03 (s, 3H) 1.11-1.19 (m, 7.09 Hz, 3H); LCMS: 99.07%; ESI MS m/z calcd. For C₁₀H₁₃FN₆O₂ ([M+H]+) 269.11; found 269.2; HPLC: 99.41%.

### Compound I-15: N,N-diethyl-3-(2-fluoroethyl)-4-oxo-3H,4H-imidazo[4,3-d][1,2,3,5]tetrazine-8-carboxamide

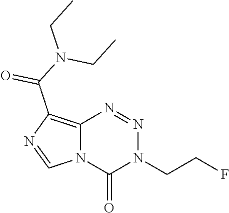 [SMILES: CCN(CC)C(=O)c1ncn2c(=O)n(CCF)nnc12]

The title compound was prepared using the general procedure on 100 mg scale to provide the title compound as a brown solid (90 mg, 72%) ¹H NMR (400 MHz, DMSO-d6) δ 8.84 (s, 1H) 4.72-4.92 (m, 2H) 4.52-4.68 (m, 2H) 3.50 (q, J=7.00 Hz, 2H) 3.39 (q, J=7.00 Hz, 2H) 1.08-1.21 (m, 6H); LCMS: 98.54%; ESI MS m/z calcd. For C₁₁H₁₅FN₆O₂ ([M+H]+) 283.13; found 283.2; HPLC: 98.80%.

### Compound I-16: 3-(2-fluoroethyl)-4-oxo-N-propyl-3H,4H-imidazo[4,3-d][1,2,3,5]tetrazine-8-carboxamide

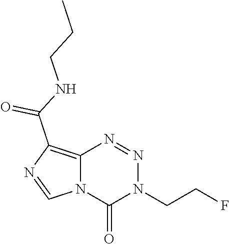 [SMILES: CCCNC(=O)c1ncn2c(=O)n(CCF)nnc12]

The title compound was prepared using the general procedure on 100 mg scale to provide the title compound as a brown solid (80 mg, 67%) ¹H NMR (400 MHz, DMSO-d6) δ 8.86 (s, 1H) 8.48 (br t, J=5.88 Hz, 1H) 4.72-4.94 (m, 2H) 4.55-4.69 (m, 2H) 3.23-3.29 (m, 2H) 1.48-1.62 (m, 2H) 0.89 (t, J=7.44 Hz, 3H); LCMS: 97.70%; ESI MS m/z calcd. For C₁₀H₁₃FN₆O₂ ([M+H]+) 269.1; found 269.2; HPLC: 98.30%.

### Compound I-17: 3-(2-fluoroethyl)-N-methyl-4-oxo-N-propyl-3H,4H-imidazo[4,3-d][1,2,3,5]tetrazine-8-carboxamide

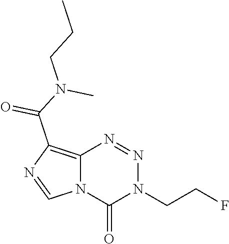 [SMILES: CCCN(C)C(=O)c1ncn2c(=O)n(CCF)nnc12]

The title compound was prepared using the general procedure on 100 mg scale to provide the title compound as a brown solid (70 mg, 56%) ¹H NMR (400 MHz, DMSO-d6) δ 8.84 (d, J=0.98 Hz, 1H) 4.88 (t, J=4.65 Hz, 1H) 4.76 (t, J=4.89 Hz, 1H) 4.63 (t, J=4.65 Hz, 1H) 4.54-4.59 (m, 1H) 3.47, 3.34 (t, J=7.34 Hz, 2H) 3.02 (d, J=1.47 Hz, 3H) 1.52-1.67 (m, 2H) 0.92, 0.72 (t, J=7.34 Hz, 3H); (Rotamers) LCMS: 95.91%; ESI MS m/z calcd. For C₁₁H₁₅FN₆O₂ ([M+H]+) 283.13; found 283.2; HPLC: 96.51%.

Compound I-18: N-ethyl-3-(2-fluoroethyl)-4-oxo-N-propyl-3H,4H-imidazo[4,3-d][1,2,3,5]tetrazine-8-carboxamide

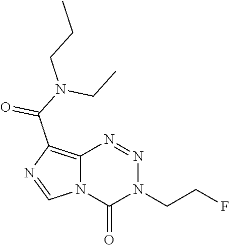 [SMILES: CCCN(CC)C(=O)c1ncn2c(=O)n(CCF)nnc12]

The title compound was prepared using the general procedure on 100 mg scale to provide the title compound as a brown solid (75 mg, 57%) ¹H NMR (400 MHz, DMSO-d6) δ 8.84 (s, 1H) 4.88 (t, J=4.82 Hz, 1H) 4.75-4.78 (m, 1H) 4.63 (t, J=4.82 Hz, 1H) 4.57 (t, J=4.82 Hz, 1H) 3.51 (q, J=7.05 Hz, 1H) 3.36-3.46 (m, 2H) 3.34 (br s, 1H) 1.53-1.68 (m, 2H) 1.07-1.21 (m, 3H) 0.70-0.96 (m, 3H); LCMS: 99.66%; ESI MS m/z calcd. For C₁₂H₁₇FN₆O₂ ([M+H]+) 297.14; found 297.2; HPLC: 98.55%.

### Example 17: Synthesis of Compound I-19: 3-(2-fluoroethyl)-N-(D3)methyl-4-oxo-3H,4H-imidazo[4,3-d][1,2,3,5]tetrazine-8-carboxamide

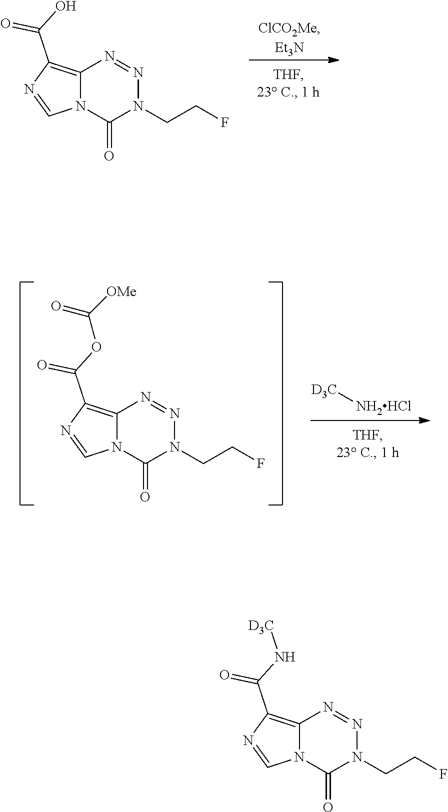

Step 1: To a solution of 3-(2-fluoroethyl)-4-oxo-3,4-dihydroimidazo[5,1-d][1,2,3,5]tetrazine-8-carboxylic acid (150 mg, 0.66 mmol, 1.0 mmol) in THF (10 mL) was added Et₃N(0.1 mL, 0.79 mmol, 1.2 eq), methylchloroformate (68.6 mg, 0.72 mmol, 1.1 eq) at room temperature and the resultant reaction mixture was stirred for 1 h.

Step 2: To this reaction mixture was added methan-d3-amine hydrochloride (70 mg, 0.99 mmol, 1.5 eq) and the resulting reaction mixture was stirred for 16 h at room temperature. After consumption of starting material as indicated by TLC, volatiles were removed under reduced pressure and crude product was purified by flash chromatography using 60-70% ethyl acetate in heptane as eluent to afford 3-(2-fluoroethyl)-N-(methyl-d3)-4-oxo-3,4-dihydroimidazo[5,1-d][1,2,3,5]tetrazine-8-carboxamide (32.6 mg, 0.134 mmol, 20%) as off white solid. ¹H NMR (400 MHz, DMSO-d6) δ 8.86 (s, 1H) 8.43 (s, 1H) 4.88 (t, J=4.82 Hz, 1H) 4.77 (t, J=4.25 Hz, 1H) 4.65 (t, J=4.82 Hz, 1H) 4.59 (t, J=5.25 Hz, 1H); LCMS: 98.15%; ESI MS m/z calcd. For C₈H₆D₃FN₆O₂ (M+H)+244.10; found 244.1; HPLC: 96.50%.

### Example 18: Synthesis of Compound I-20: N-benzyl-3-(2-fluoroethyl)-N-(2H3)methyl-4-oxo-3H,4H-imidazo[4,3-][1,2,3,5]tetrazine-8-carboxamide

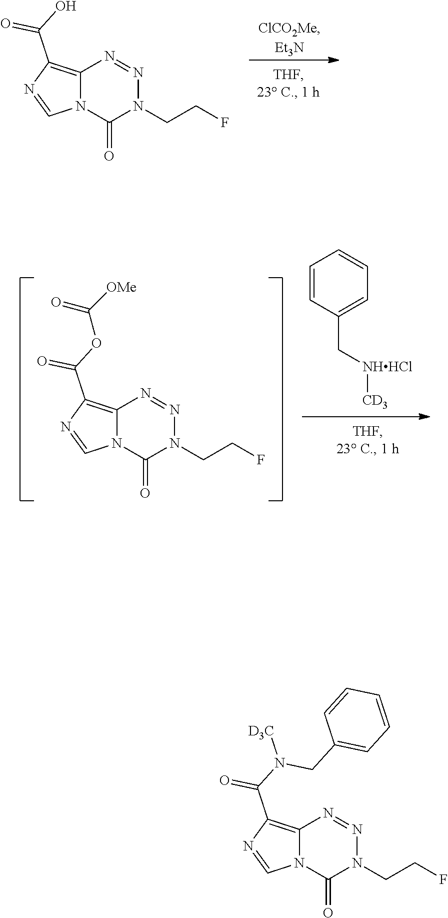

Step 1: To a solution of 3-(2-fluoroethyl)-4-oxo-3,4-dihydroimidazo[5,1-d] [1,2,3,5]tetrazine-8-carboxylic acid (50 mg, 0.22 mmol, 1 eq) in THF (5 mL, 0.04 M) were added Et₃N(0.03 mL, 0.24 mmol, 1.1 eq), methylchloroformate (0.01 mL, 0.24 mmol, 1.1 eq) at room temperature and the resultant reaction mixture was stirred for 1 h.

Step 2: To this reaction mixture was added N-benzylmethan-d3-amine hydrochloride (53 mg, 0.33 mmol, 1.5 eq) and continued stirring for 16 h at room temperature. After consumption of starting material as indicated by TLC, volatiles were removed under reduced pressure and obtained crude product was purified by flash chromatography using 60-70% ethyl acetate in heptane as eluent to afford N-benzyl-3-(2-fluoroethyl)-N-(methyl-d3)-4-oxo-3,4-dihydroimidazo[5,1-d][1,2,3,5]tetrazine-8-carboxamide (19 mg, 0.056 mmol, 26%) as off white solid. ¹H NMR (400 MHz, DMSO-d6) δ 8.86 (d, J=7.75 Hz, 1H) 7.23-7.44 (m, 5H) 4.88 (q, J=4.42 Hz, 1H) 4.73-4.79 (m, 2H) 4.68 (s, 1H) 4.64 (q, J=4.63 Hz, 1H) 4.57 (q, J=4.54 Hz, 1H); LCMS: 95.25%; ESI MS m/z calcd. For C₁₅H₁₂D₃FN₆O₂ (M+H)+334.15; found 334.1; HPLC: 97.85%.

### Example 19: Anticancer Activity Towards Human Brain Glioblastoma Cells

Exemplary compounds were evaluated for activity against LN229 human brain glioblastoma cells. Experimental procedures and results are provided elsewhere herein.

### Part I—Experimental Procedure

LN229 quad cells were maintained in Dulbecco's Modified Eagle Medium (DMEM) supplemented with 10% fetal bovine serum. Cells were plated into sterile black with glass bottom 384-well plates (Cellvis) at a concentration of 1,000 cells/well (20 μL total volume) using a MultiDrop (Thermo Fisher). Then, assay plates were centrifuged at 300 rpm for 2 seconds and incubated overnight in a 37° C. 5% CO₂ incubator. Compounds were prepared as 100 mM stocks in DMSO and stored protected from light at −20′C until use. Prior to compound addition, the stock solutions of compound were diluted two-fold serially in DMSO from 100 mM down to 0.05 mM in a 384-well Source plate. Vehicle control wells containing DMSO and positive control wells containing 10 mM bortezomib were also added to the Source plate. An aliquot in the amount of 40 nL of compound stock solution or DMSO vehicle was transferred from the source plate to the cell assay plate using an Echo Acoustic dispense (Beckman). Three replicate dilution curves of each compound were run on each assay plate. An aliquot in the amount of 120 nL of a 10 mM bortezomib solution was transferred to the positive control wells resulting in a final concentration of 60 μM. The final concentration of compounds ranged from 200 μM to 0.1 uM (12-point, 2-fold dilution dose response curve), and the final DMSO concentration was 0.2%.

Assay plates were centrifuged at 500 rpm for 2 seconds and incubated for 120 hours at 37° C. in a humidified 5% CO₂ incubator. Following incubation, cells were fixed with 4% paraformaldehyde and stained with DAPI for nuclei visualization. The images were acquired on the InCell 2200 high content imager (GE, now Molecular Devices), and quantified using InCell Analyzer image analysis software. Z prime factor, signal-to-background and coefficient of variation were calculated for each assays plate using mean and standard deviation values of the negative (vehicle) and positive (bortezomib) control wells to ensure assay robustness. Raw cell count data for test compounds was normalized to percent viability relative to the DMSO vehicle control. Data was plotted in GraphPad Prism using a variable slope 4-parameter fit.

### Part II—Results

Inhibition data for compounds tested in the assay is provided herein. The symbol “+++++” indicates a IC₅₀ less than 10 μM. The symbol “++++” indicates an IC₅₀ in the range of 10 μM to 20 μM. The symbol “+++” indicates a IC₅₀ in the range of greater than 20 μM to 50 μM. The symbol “++” indicates a IC₅₀ in the range of greater than 50 μM to 80 μM. The symbol “+” indicates a IC₅₀ greater than 80 μM. The symbol “N/A” indicates that no data was available.

TABLE 2

| | MGMT+ | MGMT+ | MGMT− | MGMT− |
|---|---|---|---|---|
| | MMR+ | MMR− | MMR+ | MMR− |
| Compound No. | Activity | Activity | Activity | Activity |
| I-2 | + | + | ++++ | +++++ |
| I-3 | + | + | +++++ | +++++ |
| I-4 | + | + | ++++ | +++++ |
| I-5 | + | + | ++++ | +++++ |
| I-6 | + | + | +++ | ++++ |
| I-7 | +++++ | +++++ | +++++ | +++++ |
| I-8 | + | + | ++++ | +++++ |
| I-9 | + | + | ++++ | +++++ |
| I-10 | + | + | +++ | +++++ |
| I-12 | + | + | +++++ | +++++ |
| I-14 | + | + | +++ | ++++ |
| I-15 | + | + | ++ | ++++ |
| I-1 | + | + | ++++ | +++++ |
| I-16 | + | + | ++++ | +++++ |
| I-17 | + | + | + | +++ |
| I-18 | + | + | + | +++ |
| I-13 | +++ | +++ | +++ | ++++ |
| I-19 | + | + | ++ | +++++ |
| I-20 | + | + | ++ | +++++ |

### Example 20: Pharmacokinetic and Brain Distribution Study in Mice

Pharmacokinetic properties and brain distribution profiles of exemplary compounds were evaluated in male C57BL/6 mice. Experimental procedures and results are provided elsewhere herein.

Study drugs were compound KL50 and compound I-1. Compound KL50 has the structure

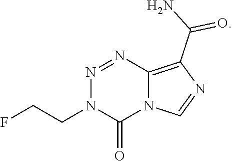 [SMILES: NC(=O)c1ncn2c(=O)n(CCF)nnc12]

Compound I-1 has the structure

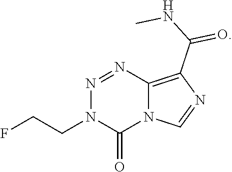 [SMILES: CNC(=O)c1ncn2c(=O)n(CCF)nnc12]

Dosing solutions of compound KL-50 and compound I-1 were prepared as follows, just prior to use in the experimental procedures.

Compound KL50 dosing solution: 3.39% DMSO+96.61% (10% HP-β-CD in saline) at 2 mg/mL;
1) Weigh 17.39 mg of compound KL50 into a clean tube.
2) Add 0.295 mL DMSO into the tube containing the compound.
3) Vortex the tube for 3 min and sonicate it for 4 min.
4) Add 8.400 mL 10% HP-β-CD in saline into the tube and vortex the tube for 3 min.
   Compound I-1 dosing solution: 3.39% DMSO+96.61% (10% HP-β-CD in saline) at 2 mg/mL;
1) Weigh 16.86 mg Compound I-1 into a clean tube.
2) Add 0.286 mL DMSO into the tube containing the compound.
3) Vortex the tube for 3 min and sonicate it for 4 min.
4) Add 8.144 mL 10% HP-β-CD in saline into the tube and vortex the tube for 3 min.

The in-vivo pharmacokinetic and brain distribution study was conducted under the following parameters. Twenty seven C57BL/6 male mice were purchased from Shanghai JiHui Laboratory Animal Co. LTD. Each mouse weighted from 16 g to 17 g. The mice were fasted overnight and fed post 4 hr sampling. Compound KL50 and Compound I-I were administered via oral gavage (PO) at a dose of 20 mg/kg (10 mL/kg). After dosing, sampling was completed at 0.083, 0.167, 0.333, 0.5, 0.75, 1, 2, 4 and 8 hr post dose, with three mice sacrificed at each time point.

Terminal bleeding for plasma and brain was completed at each time point. Approximately 110 μL blood per time point was collected into a K₂EDTA tube via the facial vein. The blood sample was put on wet ice and centrifuged to obtain plasma sample (2000 g, 5 min under 4° C.) within 15 minutes. Plasma samples were acidified with 6 μL of formic acid for every 54 μL of plasma. After blood collection, a mid-line incision was made in the animal's scalp and the skin was retracted. The skull overlying the brain was removed. Then, the whole brain was collected, rinsed with cold saline, dried on filtrate paper, and weighed; and then snap frozen by placing the brain into dry-ice. Plasma samples were stored at approximately −70° C. until analysis. Brain tissue was homogenized with 3 volumes (v/w) of PBS (1% FA) and then analyzed with LC-MS/MS. The concentration of study drug was corrected with a dilution factor of four as following: Brain concentration=brain homogenate conc.×4, assuming 1 g wet brain tissue equals to 1 mL

Results for compound KL50 are provided in Tables 3-7 and FIG. 4, where FIG. 4 shows mean plasma concentration-time profiles and brain concentration-time profiles of KL-50 after single PO dose at 20 mg/kg in male C57BL/6 mice (N=3/timepoint). The plasma PK parameters in Table 4 were calculated based on the plasma concentration values in Table 3. The brain PK parameters in Table 6 were calculated based on the brain concentration values in Table 5.

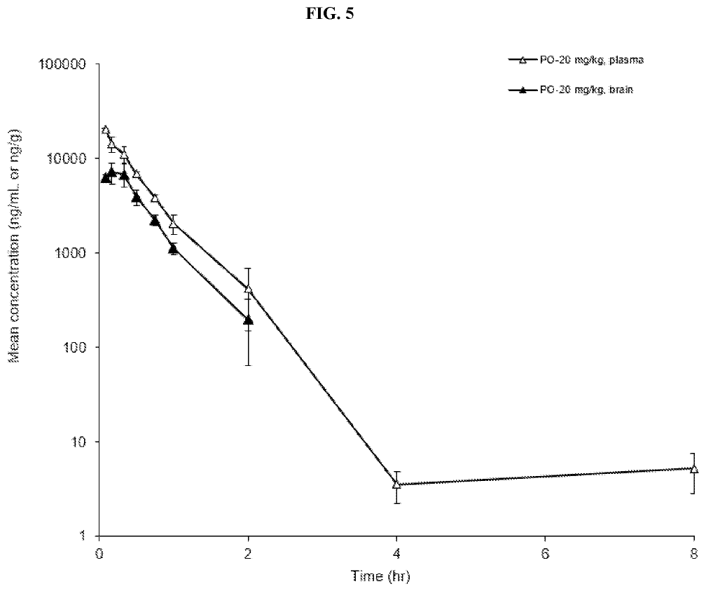

Results for compound I-1 are provided in Tables 8-12 and FIG. 5, where FIG. 5 shows mean plasma concentration-time profiles and brain concentration-time profiles of compound I-1 after single PO dose at 20 mg/kg in male C57BL/6 mice (N=3/timepoint). The plasma PK parameters in Table 9 were calculated based on the plasma concentration values in Table 8. The brain PK parameters in Table 11 were calculated based on the brain concentration values in Table 10.

TABLE 3
Individual and mean plasma concentration-time data of
KL50 after a PO dose at 20 mg/kg in male C57BL/6 mice

| Sampling | | | | | | |
|---|---|---|---|---|---|---|
| Time | KL50 Concentration (ng/mL) | | | Mean | | |
| (hr) | Individuals | | | (ng/mL) | SD | CV(%) |
| 0.083 | 22300 | 25600 | 28000 | 25300 | 2862 | 11.3 |
| 0.167 | 24500 | 22900 | 18100 | 21833 | 3331 | 15.3 |
| 0.333 | 19000 | 14500 | 20900 | 18133 | 3287 | 18.1 |
| 0.5 | 14400 | 12400 | 15300 | 14033 | 1484 | 10.6 |
| 0.75 | 9590 | 11000 | 10200 | 10263 | 707 | 6.89 |
| 1 | 10500 | 7340 | 12300 | 10047 | 2511 | 25.0 |
| 2 | 2580 | 2750 | 4570 | 3300 | 1103 | 33.4 |
| 4 | 1060 | 1590 | 796 | 1149 | 404 | 35.2 |
| 8 | 305 | 104 | 185 | 198 | 101 | 51.1 |

TABLE 4
Plasma PK Data of KL50 after a PO dose
at 20 mg/kg in male C57BL/6 mice

| PK parameters | Value |
|---|---|
| Rsq_adjusted | 0.996 |
| Tₘₐₓ (hr) | 0.083 |
| Cₘₐₓ (ng/mL) | 25300 |
| Regression points (hr) | 2~8 |
| T_{1/2} (hr) | 1.49 |
| AUCₗₐₛₜ (hr*ng/ml) | 28424 |
| AUC_{INF} (hr*ng/mL) | 28850 |
| MRT_{INF} (hr) | 1.47 |

TABLE 5
Individual and mean brain concentration-time data of KL50
after single PO dose at 20 mg/kg in male C57BL/6 mice

| Sampling | | | | | | |
|---|---|---|---|---|---|---|
| Time | KL50 Concentration (ng/g) | | | Mean | | |
| (hr) | Individuals | | | (ng/g) | SD | CV(%) |
| 0.083 | 1540 | 1970 | 1940 | 1817 | 240 | 13.2 |
| 0.167 | 3180 | 2810 | 2820 | 2937 | 211 | 7.18 |
| 0.333 | 3630 | 2980 | 4450 | 3687 | 737 | 20 |
| 0.5 | 3730 | 3230 | 3830 | 3597 | 321 | 8.94 |
| 0.75 | 3300 | 3060 | 3380 | 3247 | 167 | 5.13 |
| 1 | 2920 | 2530 | 3270 | 2907 | 370 | 12.7 |
| 2 | 934 | 953 | 1530 | 1139 | 339 | 29.7 |
| 4 | 184 | 427 | 199 | 270 | 136 | 50.4 |
| 8 | 85.3 | 18.2 | 45.2 | 49.6 | 33.8 | 68.1 |

TABLE 6
Brain PK Data for KL50 after single PO
dose at 20 mg/kg in male C57BL/6 mice

| PK parameters | Value |
|---|---|
| Rsq_adjusted | 0.972 |
| Tₘₐₓ (hr) | 0.333 |
| Cₘₐₓ (ng/g) | 3687 |
| Regression points (hr) | 0.5~8 |
| T_{1/2} (hr) | 1.18 |
| AUCₗₐₛₜ (hr*ng/g) | 7128 |
| AUC_{INF} (hr*ng/g) | 7213 |
| MRT_{INF} (hr) | 1.57 |
| AUC_{brain}/AUCₚₗₐₛₘₐ | 25.1 |

TABLE 7
Brain Concentration to Plasma Concentration Ratio of KL50
after single PO dose at 20 mg/kg in male C57BL/6 mice

| Sampling | Brain Concentration/ | | | | | |
|---|---|---|---|---|---|---|
| Time | Plasma Concentration | | | | | |
| (hr) | Individuals | | | Mean | SD | CV(%) |
| 0.083 | 0.0691 | 0.0770 | 0.0693 | 0.0718 | 0.00449 | 6.26 |
| 0.167 | 0.130 | 0.123 | 0.156 | 0.136 | 0.0174 | 12.8 |
| 0.333 | 0.191 | 0.206 | 0.213 | 0.203 | 0.0111 | 5.47 |
| 0.5 | 0.259 | 0.260 | 0.250 | 0.257 | 0.00549 | 2.14 |
| 0.75 | 0.344 | 0.278 | 0.331 | 0.318 | 0.0350 | 11.0 |
| 1 | 0.278 | 0.345 | 0.266 | 0.296 | 0.0424 | 14.3 |
| 2 | 0.362 | 0.347 | 0.335 | 0.348 | 0.0137 | 3.93 |
| 4 | 0.174 | 0.269 | 0.250 | 0.231 | 0.0503 | 21.8 |
| 8 | 0.280 | 0.175 | 0.244 | 0.233 | 0.0532 | 22.9 |

TABLE 8
Individual and mean plasma concentration-time data of Compound
I-1 after a PO dose at 20 mg/kg in male C57BL/6 mice

| Sam- | | | | | | |
|---|---|---|---|---|---|---|
| pling | Compound I-1 | | | | | |
| Time | Concentration (ng/mL) | | | Mean | | CV |
| (hr) | Individuals | | | (ng/mL) | SD | (%) |
| 0.083 | 21200 | 20300 | 20500 | 20667 | 473 | 2.29 |
| 0.167 | 11300 | 15000 | 16600 | 14300 | 2718 | 19.0 |
| 0.333 | 8830 | 11300 | 13400 | 11177 | 2287 | 20.5 |
| 0.5 | 6300 | 7050 | 7190 | 6847 | 479 | 6.99 |
| 0.75 | 3980 | 4010 | 3430 | 3807 | 327 | 8.58 |
| 1 | 2180 | 1520 | 2430 | 2043 | 470 | 23.0 |
| 2 | 359 | 714 | 185 | 419 | 270 | 64.3 |
| 4 | 5.01 | 2.42 | 3.21 | 3.55 | 1.33 | 37.4 |
| 8 | 2.65 | 5.63 | 7.33 | 5.20 | 2.37 | 45.5 |

TABLE 9
Plasma PK Parameters of Compound I-1 after
a PO dose at 20 mg/kg in male C57BL/6 mice

| PK parameters | Value |
|---|---|
| Rsq_adjusted | 0.778 |
| Tₘₐₓ (hr) | 0.083 |
| Cₘₐₓ (ng/mL) | 20667 |
| Regression points (hr) | 0.167~8 |
| T_{1/2} (hr) | 0.621 |
| AUCₗₐₛₜ (hr*ng/ml) | 9680 |
| AUC_{INF} (hr*ng/mL) | 9685 |
| MRT_{INF} (hr) | 0.536 |

TABLE 10
Individual and mean brain concentration-time data of Compound
I-1 after single PO dose at 20 mg/kg in male C57BL/6 mice

| Sampling | Compound I-1 | | | | | |
|---|---|---|---|---|---|---|
| Time | Concentration (ng/g) | | | Mean | | |
| (hr) | Individuals | | | (ng/g) | SD | CV(%) |
| 0.083 | 5860 | 6300 | 6740 | 6300 | 440 | 6.98 |
| 0.167 | 5420 | 7100 | 9110 | 7210 | 1847 | 25.6 |
| 0.333 | 4950 | 6850 | 8730 | 6843 | 1890 | 27.6 |
| 0.5 | 3310 | 4770 | 3750 | 3943 | 749 | 19.0 |
| 0.75 | 2290 | 2500 | 1920 | 2237 | 294 | 13.1 |
| 1 | 1140 | 952 | 1280 | 1124 | 165 | 14.6 |
| 2 | 155 | 345 | 90.6 | 197 | 132 | 67.2 |
| 4* | BQL | BQL | BQL | BQL | NA | NA |
| 8 | BQL | BQL | BQL | BQL | NA | NA |

*BQL: Below the quantifiable limit of 1.00 ng/mL or ng/g for Compound I-1 in mouse plasma and brain homogenate.

TABLE 11
Brain PK Parameters of Compound I-1 after single
PO dose at 20 mg/kg in male C57BL/6 mice

| PK parameters | Value |
|---|---|
| Rsq_adjusted | 0.987 |
| Tₘₐₓ (hr) | 0.167 |
| Cₘₐₓ (ng/g) | 7210 |
| Regression points (hr) | 0.5~2 |
| T_{1/2} (hr) | 0.352 |
| AUCₗₐₛₜ (hr*ng/g) | 4749 |
| AUC_{INF} (hr*ng/g) | 4849 |
| MRT_{INF} (hr) | 0.527 |
| AUC_{brain}/AUCₚₗₐₛₘₐ | 49.1 |

TABLE 12
Brain Concentration to Plasma Concentration Ratio of Compound
I-1 after single PO dose at 20 mg/kg in male C57BL/6 mice

| Sampling | Brain Concentration/ | | | | | |
|---|---|---|---|---|---|---|
| Time | Plasma Concentration | | | | | |
| (hr) | Individuals | | | Mean | SD | CV(%) |
| 0.083 | 0.276 | 0.310 | 0.329 | 0.305 | 0.0266 | 8.70 |
| 0.167 | 0.480 | 0.473 | 0.549 | 0.501 | 0.0419 | 8.36 |
| 0.333 | 0.561 | 0.606 | 0.651 | 0.606 | 0.0455 | 7.50 |
| 0.5 | 0.525 | 0.677 | 0.522 | 0.575 | 0.0884 | 15.4 |
| 0.75 | 0.575 | 0.623 | 0.560 | 0.586 | 0.0332 | 5.66 |
| 1 | 0.523 | 0.626 | 0.527 | 0.559 | 0.0586 | 10.5 |
| 2 | 0.432 | 0.483 | 0.490 | 0.468 | 0.0318 | 6.78 |
| 4 | NA | NA | NA | NA | NA | NA |
| 8 | NA | NA | NA | NA | NA | NA |

### Example 21: Evaluation of Anti-cancer Activity of Exemplary Compounds in Mice

Having Tumors Formed from LN-229 Human Brain Glioblastoma Cells Exemplary compounds were evaluated for anti-cancer activity by administration to mice having tumors formed from LN-229 human brain glioblastoma cells. Experimental procedures and results are provided elsewhere herein.

### Part I—Experimental Procedures

Mice bearing tumors formed from LN-229 human brain glioblastoma cells were prepared as follows: LN229 MGMT−/MMR− cells stably expressing firefly luciferase (lentivirus-plasmids from Cellomics Technology; PLV-10003) were injected intracranially into mice using a stereotactic injector. For this procedure, 1.5 million LN229 MGMT−/MMR− cells in 5 μl PBS were injected into the brain of the mice subjects, and then the mice were imaged weekly using the IVIS Spectrum In Vivo Imaging System (PerkinElmer) according to the manufacturer's protocol. Images were taken on a weekly basis and acquired 10 min post intraperitoneal injection with d-luciferin (150 mg/kg of animal mass). Tumors were allowed to grow to an average of 1.0×10⁸ RLU before randomization of the mice.

Treatments using study compounds were administered PO (oral gavage), with 10% cyclodextrin vehicle control or study compound, according to the indicated treatment regimen. Animals were observed daily, with weekly tumor imaging and body weight measurements. Quantification of BLI flux (photons/sec) was made through the identification of a region of interest (ROI) for each tumor. Study compounds were those listed in the following table:

TABLE 13
Study Compounds

| Compound Identifier | Chemical Structure |
|---|---|
| I-1 | 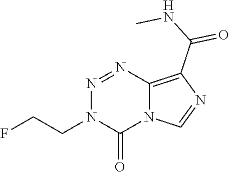 [SMILES: CNC(=O)c1ncn2c(=O)n(CCF)nnc12] |
| KL50 | 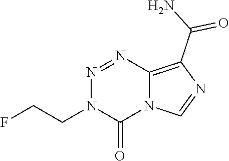 [SMILES: NC(=O)c1ncn2c(=O)n(CCF)nnc12] |
| TMZ | 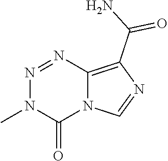 |

Study compounds were administered to mice at a dose of 25 mg/kg PO for 5 days; thereafter no further study compound was administered to the mice.

Statistical analysis of data was performed using GraphPad Prism software. Data was presented as mean±SEM. For xenograft growth delay experiments, comparisons were made with ordinary two-way ANOVA with Tukey's multiple comparisons test, with individual variances computed for each comparison. For xenograft survival analysis, Kaplan-Meier analysis was used to evaluate survival rate based on death or removal from study when body weight loss exceeded 20% of initial body weight, and statistical comparisons were made by log-rank (Mantel-Cox) test with Bonferroni correction for multiple comparisons.

### Part II—Results

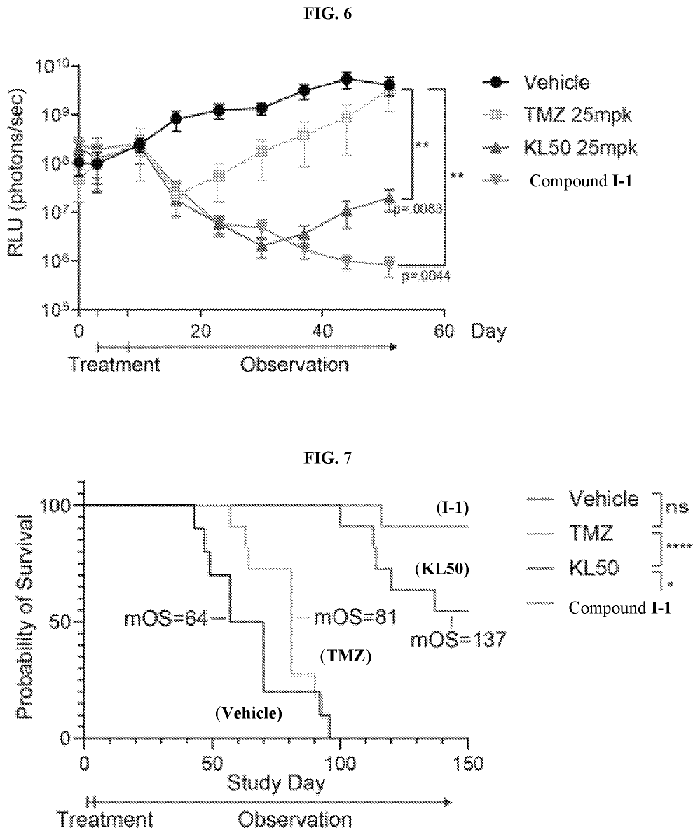

Results from the experiment are depicted in FIGS. 6 and 7. FIG. 6 shows the results of bioluminescence imaging for each group of mice according to study compound or vehicle. FIG. 7 shows survival endpoint data for each group of mice according to study compound or vehicle. The data show that mice treated with Compound I-1 had a longer duration of survival in this experiment than mice treated with any of vehicle, TMZ, or KL50.

All references cited herein are incorporated herein by reference as if included in their entirety.

In the description and claims of this specification the word “comprise” and variations of that word, such as “comprises” and “comprising” are not intended to exclude other features, additives, components, integers or steps but rather, unless otherwise stated explicitly, the scope of these words should be construed broadly such that they have an inclusive meaning rather than an exclusive one.

Although the compounds, compositions, and methods of the invention have been described in the present disclosure by way of illustrative examples, it is to be understood that the invention is not limited thereto and that variations can be made as known by those skilled in the art without departing from the teachings of the invention defined by the appended claims.

### ENUMERATED EMBODIMENTS

In some aspects, the present disclosure is directed to the following non-limiting embodiments:

Embodiment 1: A compound of formula (I):

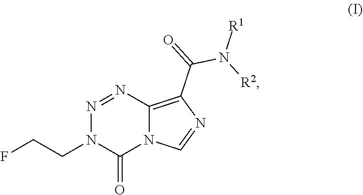

or a pharmaceutically acceptable salt thereof, wherein: R¹ and R² are each independently selected from H and lower alkyl; or R¹ and R² combine to form —(CH₂)ₙ—; and n is 2, 3, 4, or 5; provided that R¹ and R² are not simultaneously H.

Embodiment 2: The compound of Embodiment 1, wherein the compound is a compound of formula (I).

Embodiment 3: The compound of Embodiment 1 or 2, wherein R¹ is H, and wherein R² is lower alkyl.

Embodiment 4: The compound of Embodiment 1 or 2, wherein R¹ and R² are each independently lower alkyl.

Embodiment 5: The compound of Embodiment 1, 2, or 4, wherein R¹ and R² are each methyl.

Embodiment 6: The compound of Embodiment 1 or 2, wherein R¹ and R² combine to form —(CH₂)ₙ—.

Embodiment 7: The compound of Embodiment 1 or 3, wherein the compound is

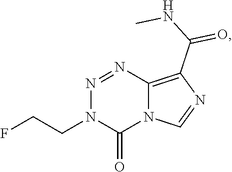 [SMILES: CNC(=O)c1ncn2c(=O)n(CCF)nnc12]

or a pharmaceutically acceptable salt thereof.

Embodiment 8: The compound of Embodiment 1 or 3, wherein the compound is

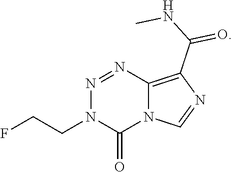 [SMILES: CNC(=O)c1ncn2c(=O)n(CCF)nnc12]

Embodiment 9: The compound of any of the preceding Embodiments, wherein the compound is selected from the group consisting of

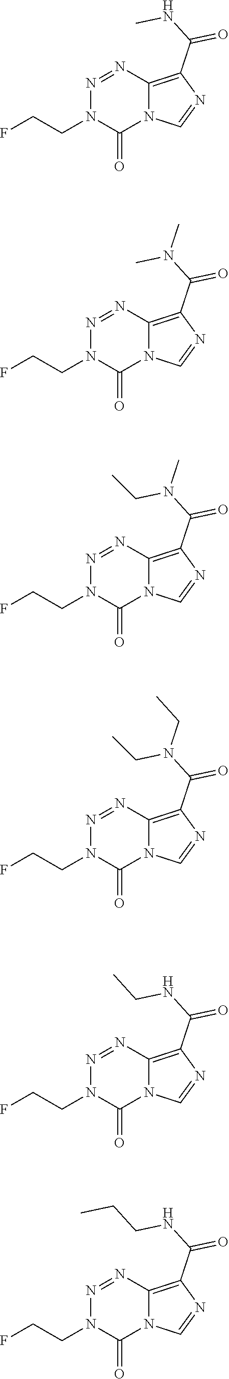 [SMILES: CNC(=O)c1ncn2c(=O)n(CCF)nnc12 ; CN(C)C(=O)c1ncn2c(=O)n(CCF)nnc12 ; CCN(C)C(=O)c1ncn2c(=O)n(CCF)nnc12 ; CCN(CC)C(=O)c1ncn2c(=O)n(CCF)nnc12 ; CCNC(=O)c1ncn2c(=O)n(CCF)nnc12 ; CCCNC(=O)c1ncn2c(=O)n(CCF)nnc12]

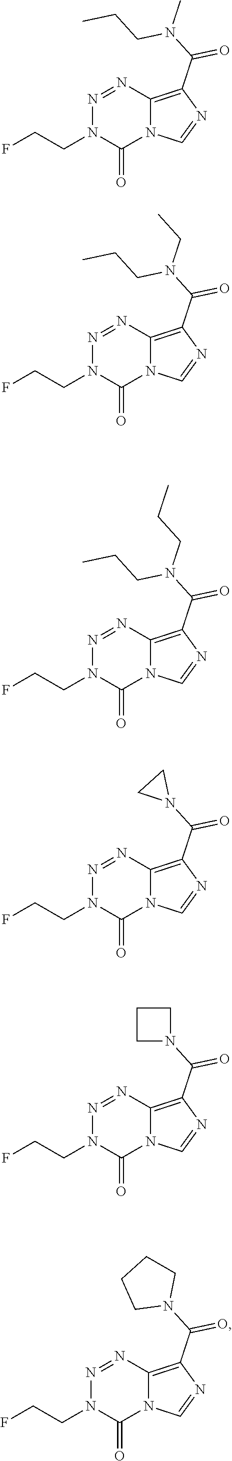 [SMILES: CCCN(C)C(=O)c1ncn2c(=O)n(CCF)nnc12 ; CCCN(CC)C(=O)c1ncn2c(=O)n(CCF)nnc12 ; CCCN(CCC)C(=O)c1ncn2c(=O)n(CCF)nnc12 ; O=C(c1ncn2c(=O)n(CCF)nnc12)N1CC1 ; O=C(c1ncn2c(=O)n(CCF)nnc12)N1CCC1 ; O=C(c1ncn2c(=O)n(CCF)nnc12)N1CCCC1]

or a pharmaceutically acceptable salt thereof.

Embodiment 10: The compound ofany of the preceding Embodiments, wherein the compound is selected from the group consisting of

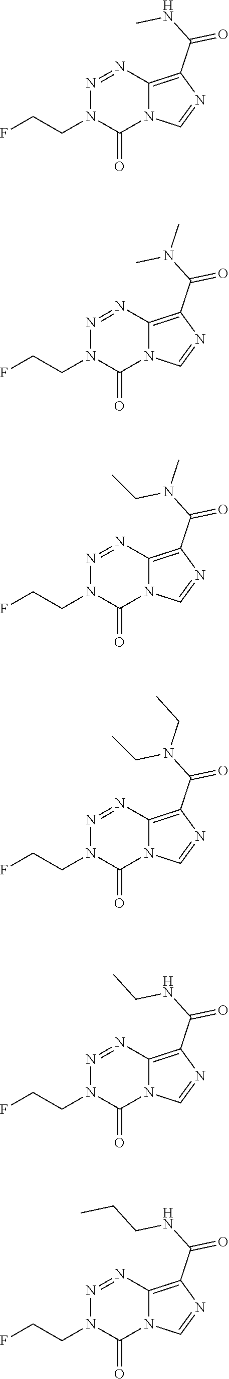 [SMILES: CNC(=O)c1ncn2c(=O)n(CCF)nnc12 ; CN(C)C(=O)c1ncn2c(=O)n(CCF)nnc12 ; CCN(C)C(=O)c1ncn2c(=O)n(CCF)nnc12 ; CCN(CC)C(=O)c1ncn2c(=O)n(CCF)nnc12 ; CCNC(=O)c1ncn2c(=O)n(CCF)nnc12 ; CCCNC(=O)c1ncn2c(=O)n(CCF)nnc12]

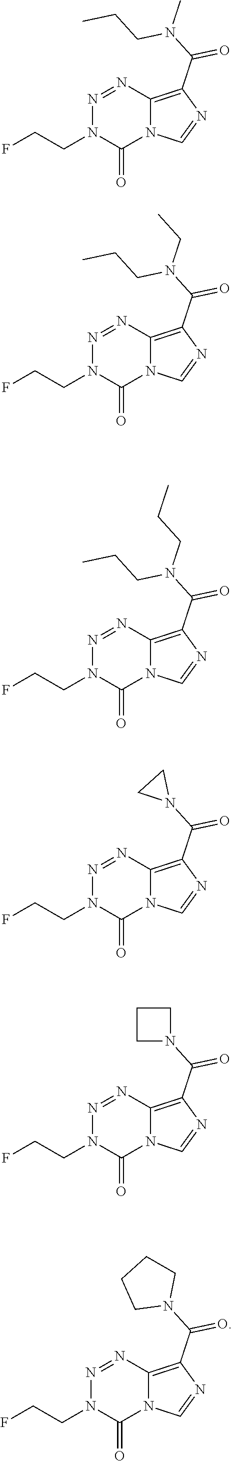 [SMILES: CCCN(C)C(=O)c1ncn2c(=O)n(CCF)nnc12 ; CCCN(CC)C(=O)c1ncn2c(=O)n(CCF)nnc12 ; CCCN(CCC)C(=O)c1ncn2c(=O)n(CCF)nnc12 ; O=C(c1ncn2c(=O)n(CCF)nnc12)N1CC1 ; O=C(c1ncn2c(=O)n(CCF)nnc12)N1CCC1 ; O=C(c1ncn2c(=O)n(CCF)nnc12)N1CCCC1]

Embodiment 11: A pharmaceutical composition comprising a compound of any one of Embodiments 1-6 and a pharmaceutically acceptable carrier.

Embodiment 12: A pharmaceutical composition comprising a compound of Embodiment 7 or 8 and a pharmaceutically acceptable carrier.

Embodiment 13: A pharmaceutical composition comprising a compound of Embodiment 9 or 10 and a pharmaceutically acceptable carrier.

Embodiment 14: A compound of formula (II):

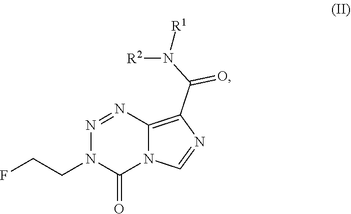

or a pharmaceutically acceptable salt thereof, wherein: R¹ is selected from the group consisting of H and lower alkyl; R² is selected from the group consisting of H, lower alkyl, trifluoroethyl,

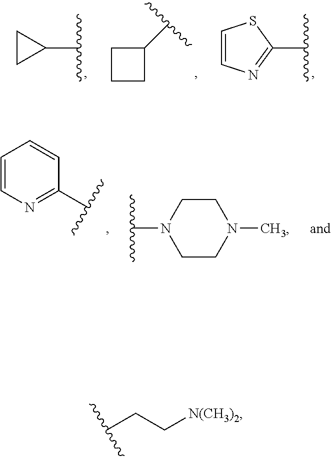

provided that R¹ is H when R² is other than H or lower alkyl; or R¹ and R² may combine to form —(CH₂)ₙ— or —(CH₂)₂—N(CH₃)—(CH₂)₂—; n is 3, 4, or 5; and provided that R¹ and R² are not both H.

Embodiment 15: The compound of Embodiment 14, wherein the compound is a compound of formula (II).

Embodiment 16: The compound of Embodiment 14 or 15, wherein R¹ is H, and R² is trifluoroethyl,

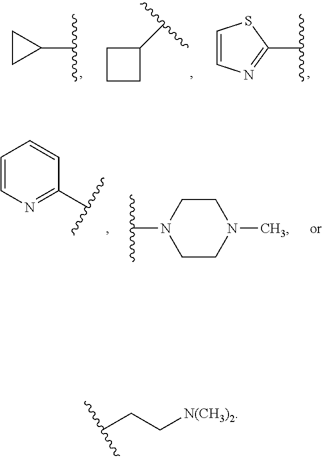

Embodiment 17: The compound of Embodiment 14, wherein the compound is selected from the group consisting of:

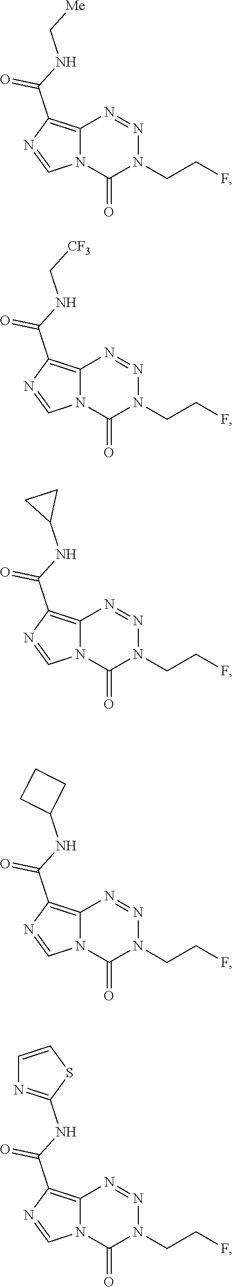 [SMILES: CCNC(=O)c1ncn2c(=O)n(CCF)nnc12 ; O=C(NCC(F)(F)F)c1ncn2c(=O)n(CCF)nnc12 ; O=C(NC1CC1)c1ncn2c(=O)n(CCF)nnc12 ; O=C(NC1CCC1)c1ncn2c(=O)n(CCF)nnc12 ; O=C(Nc1nccs1)c1ncn2c(=O)n(CCF)nnc12]

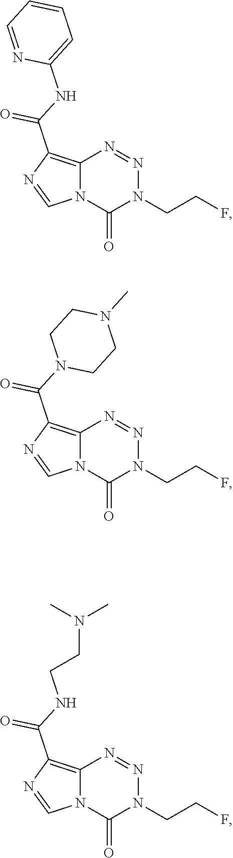 [SMILES: O=C(Nc1ccccn1)c1ncn2c(=O)n(CCF)nnc12 ; CN1CCN(C(=O)c2ncn3c(=O)n(CCF)nnc23)CC1 ; CN(C)CCNC(=O)c1ncn2c(=O)n(CCF)nnc12]

or a pharmaceutically acceptable salt thereof.

Embodiment 18: A compound selected from the group consisting of

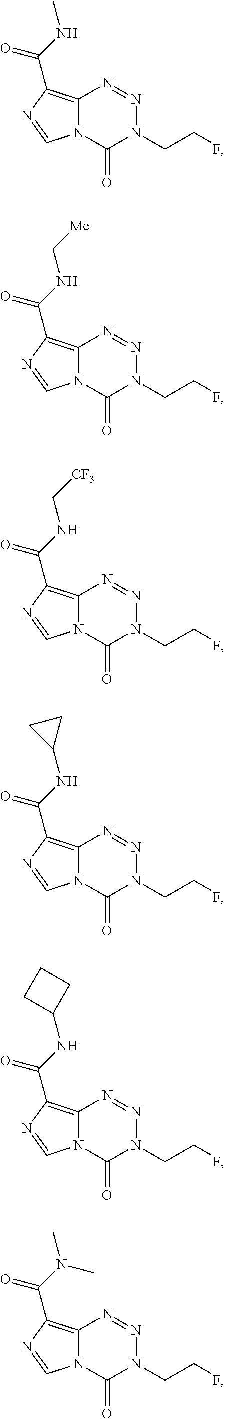 [SMILES: CNC(=O)c1ncn2c(=O)n(CCF)nnc12 ; CCNC(=O)c1ncn2c(=O)n(CCF)nnc12 ; O=C(NCC(F)(F)F)c1ncn2c(=O)n(CCF)nnc12 ; O=C(NC1CC1)c1ncn2c(=O)n(CCF)nnc12 ; O=C(NC1CCC1)c1ncn2c(=O)n(CCF)nnc12 ; CN(C)C(=O)c1ncn2c(=O)n(CCF)nnc12]

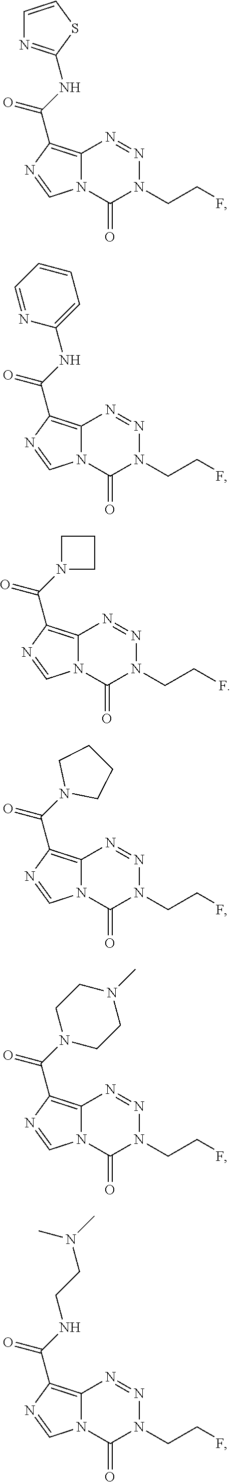

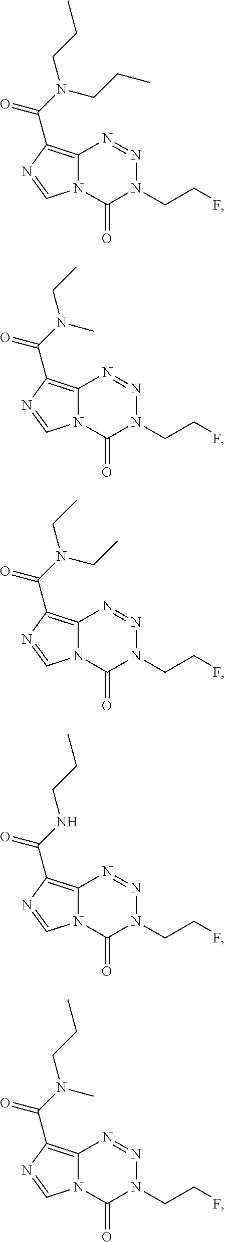

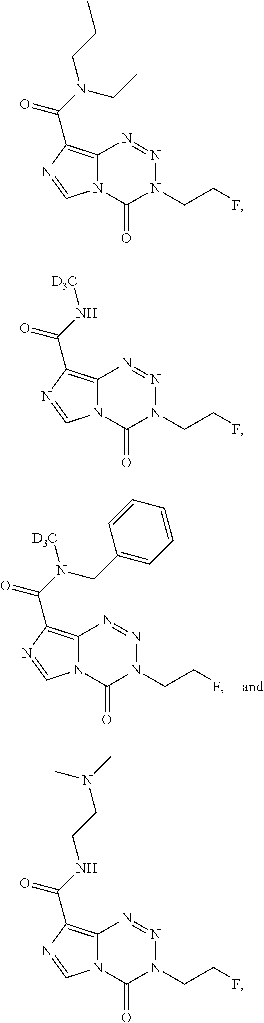

or a pharmaceutically acceptable salt thereof.

Embodiment 19: A pharmaceutical composition comprising a compound of any one of Embodiments 14-18 and a pharmaceutically acceptable carrier.

Embodiment 20: A method of treating, preventing, and/or ameliorating cancer in a subject in need thereof, the method comprising administering to the subject a therapeutically effective amount of a compound of any one of Embodiments 1-10 and 14-18, or a pharmaceutically acceptable salt thereof.

Embodiment 21: A method of treating, preventing, and/or ameliorating cancer in a subject in need thereof, the method comprising administering to the subject a therapeutically effective amount of a compound of any one of Embodiments 1-10 and 14-18, wherein the cancer is MGMT deficient and either MMR deficient or refractory to treatment with temozolomide.

Embodiment 22: The method of Embodiment 20 or 21, wherein the compound is a compound of any one of Embodiments 1-10.

Embodiment 23: The method of Embodiment 20 or 21, wherein the compound is a compound of Embodiment 7 or 8.

Embodiment 24: The method of any one of Embodiments 20-23, wherein the cancer is a solid tumor, leukemia, or lymphoma.

Embodiment 25: The method of any one of Embodiments 20-23, wherein the cancer is a solid tumor.

Embodiment 26: The method of any one of Embodiments 20-23, wherein the cancer is a brain tumor.

Embodiment 27: The method of any one of Embodiments 20-23, wherein the cancer is urothelial cancer, breast invasive carcinoma, colon adenocarcinoma, head and neck tumor (SCC), lung adenocarcinoma, rectum adenocarcinoma, acute myeloid leukemia, glioblastoma multiforme, or brain lower grade glioma.

Embodiment 28: The method of any one of Embodiments 20-23, wherein the cancer is glioma, colorectal cancer, non-small cell lung cancer, small cell lung cancer, sarcoma, pancreatic cancer, neuroendocrine tumor, esophageal cancer, lymphoma, head & neck cancer, breast cancer, bladder cancer, or leukemia.

Embodiment 29: The method of any one of Embodiments 20-23, wherein the cancer is glioblastoma multiforme.

Embodiment 30: A method of treating, preventing, and/or ameliorating cancer in a subject in need thereof, wherein the method comprises administering to the subject an agent that induces DNA lesions in the cell that lead to irreparable DNA damage to selectively treat the cancer, wherein the cancer is characterized by a cancer cell having altered MGMT activity.

Embodiment 31: A method of treating, preventing, and/or ameliorating cancer in a subject in need thereof, wherein the method comprises administering to the subject an agent that induces DNA lesions in the cell that lead to irreparable DNA damage to selectively treat the cancer, wherein the cancer is characterized by a cancer cell having altered MGMT expression.

Embodiment 32: The method of Embodiment 30 or 31, wherein the irreparable DNA damage is an unrepaired lesion.

Embodiment 33: The method of Embodiment 32, wherein the unrepaired lesion is a DNA inter- or intra-strand crosslink.

Embodiment 34: The method of any one of Embodiments 30-33, wherein the agent does not affect MGMT proficient tissue.

Embodiment 35: The method of any one of Embodiments 30-34, wherein the agent activity is independent of MMR protein expression and/or functional activity of the MMR pathway.

Embodiment 36: The method of any one of Embodiments 30-35, wherein the cancer is selected from the group consisting of a glioma, colorectal cancer, non-small cell lung cancer, small cell lung cancer, sarcoma, pancreatic cancer, neuroendocrine tumor, esophageal cancer, lymphoma, head & neck cancer, breast cancer, bladder cancer, and leukemia.

Embodiment 37: The method of any one of Embodiments 30-35, wherein the cancer is selected from the group consisting of an anaplastic astrocytoma, anaplastic oligodendroglioma, anaplastic ependymoma, medulloblastoma, and glioblastoma.

Embodiment 38: The method of any one of Embodiments 30-36, wherein the DNA lesion is a DNA double-strand break, a single-strand break, a stalled replication fork, a bulky adduct, or a lesion that further chemically reacts to form irreparable DNA damage.

Embodiment 39: The method of Embodiment 38, wherein the irreparable DNA damage is an unrepaired lesion, optionally wherein the unrepaired lesion is a DNA inter- or intra-strand crosslink.

Embodiment 40: The method of any one of Embodiments 30-39, wherein the subject is resistant to treatment with an antineoplastic agent.

Embodiment 41: The method of Embodiment 40, where the antineoplastic agent is selected from temozolomide, procarbazine, altretamine, dacarbazine, mitozolomide, cisplatin, carboplatin, dicycloplatin, eptaplatin, lobaplatin, oxaliplatin, miriplatin, nedaplatin, triplatin tetranitrate, phenanthriplatin, picoplatin, satraplatin, and lomustine.

Embodiment 42: The method of any one of Embodiments 30-41, wherein the agent is a compound of any one of Embodiments 14-18.

Embodiment 43: The method of any one of Embodiments 30-41, wherein the agent is an imidazotetrazine-based compound or a triazine-based compound.

1. A compound of formula (I): 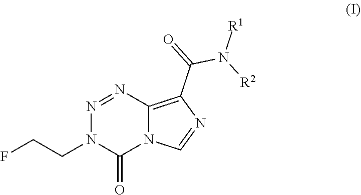
or optionally a pharmaceutically acceptable salt thereof,
wherein:
R¹ and R² are each independently selected from H and lower alkyl;
or R¹ and R² combine to form —(CH₂)ₙ—; and
n is 2, 3, 4, or 5;
provided that R¹ and R² are not simultaneously H.

2. (canceled)

3. The compound of claim 1, wherein at least one of the following applies:
i. R¹ is H, and wherein R² is lower alkyl,
ii. R¹ and R² are each independently lower alkyl;
iii. R¹ and R² are each methyl:
iv. R¹ and R² combine to form —(CH₂)ₙ—.

4-6. (canceled)

7. The compound of claim 1, wherein the compound is 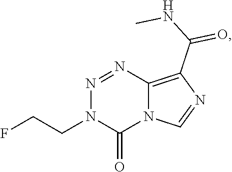 [SMILES: CNC(=O)c1ncn2c(=O)n(CCF)nnc12]
or optionally a pharmaceutically acceptable salt thereof.

8. (canceled)

9. The compound of claim 1, wherein the compound is selected from the group consisting of: 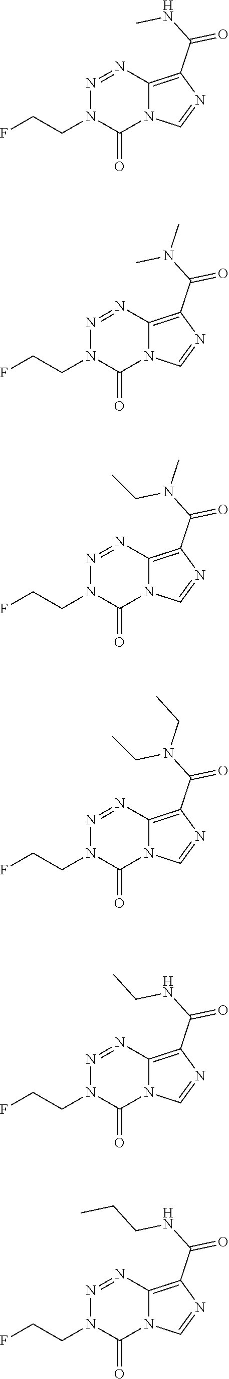 [SMILES: CNC(=O)c1ncn2c(=O)n(CCF)nnc12 ; CN(C)C(=O)c1ncn2c(=O)n(CCF)nnc12 ; CCN(C)C(=O)c1ncn2c(=O)n(CCF)nnc12 ; CCN(CC)C(=O)c1ncn2c(=O)n(CCF)nnc12 ; CCNC(=O)c1ncn2c(=O)n(CCF)nnc12 ; CCCNC(=O)c1ncn2c(=O)n(CCF)nnc12] 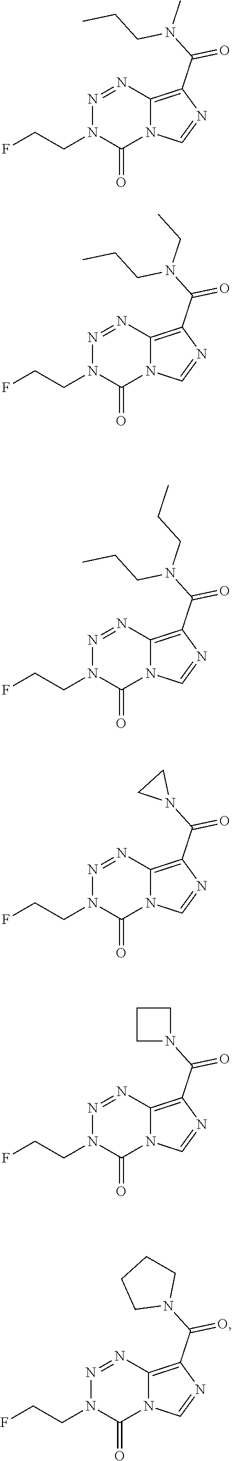 [SMILES: CCCN(C)C(=O)c1ncn2c(=O)n(CCF)nnc12 ; CCCN(CC)C(=O)c1ncn2c(=O)n(CCF)nnc12 ; CCCN(CCC)C(=O)c1ncn2c(=O)n(CCF)nnc12 ; O=C(c1ncn2c(=O)n(CCF)nnc12)N1CC1 ; O=C(c1ncn2c(=O)n(CCF)nnc12)N1CCC1 ; O=C(c1ncn2c(=O)n(CCF)nnc12)N1CCCC1]
or a pharmaceutically acceptable salt thereof.

10. The compound of claim 1, wherein the compound is selected from the group consisting of:

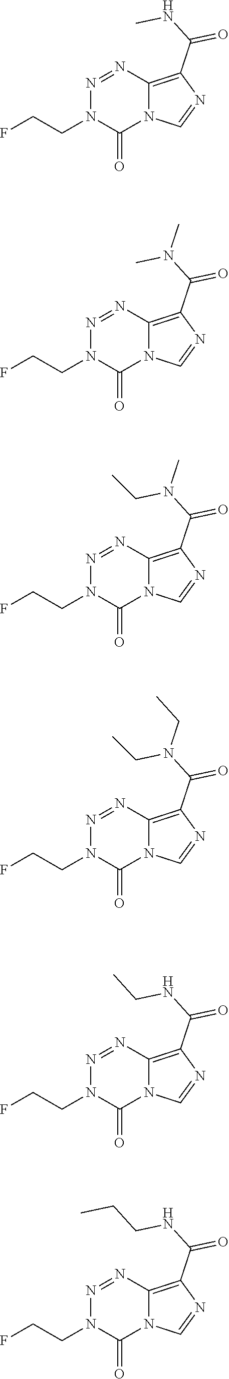 [SMILES: CNC(=O)c1ncn2c(=O)n(CCF)nnc12 ; CN(C)C(=O)c1ncn2c(=O)n(CCF)nnc12 ; CCN(C)C(=O)c1ncn2c(=O)n(CCF)nnc12 ; CCN(CC)C(=O)c1ncn2c(=O)n(CCF)nnc12 ; CCNC(=O)c1ncn2c(=O)n(CCF)nnc12 ; CCCNC(=O)c1ncn2c(=O)n(CCF)nnc12]

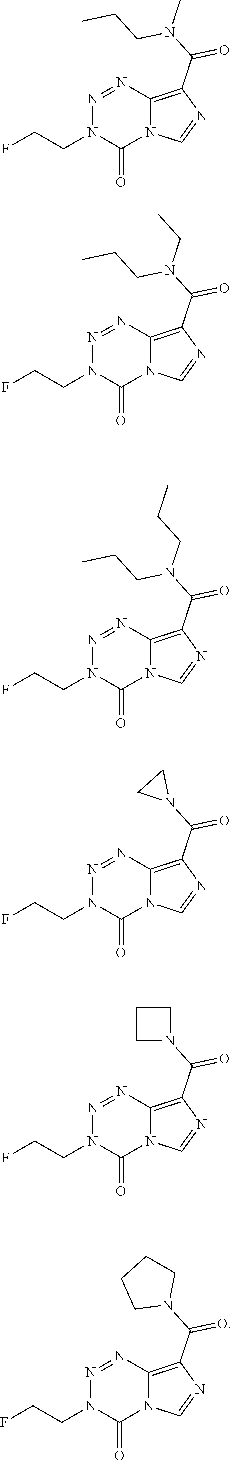 [SMILES: CCCN(C)C(=O)c1ncn2c(=O)n(CCF)nnc12 ; CCCN(CC)C(=O)c1ncn2c(=O)n(CCF)nnc12 ; CCCN(CCC)C(=O)c1ncn2c(=O)n(CCF)nnc12 ; O=C(c1ncn2c(=O)n(CCF)nnc12)N1CC1 ; O=C(c1ncn2c(=O)n(CCF)nnc12)N1CCC1 ; O=C(c1ncn2c(=O)n(CCF)nnc12)N1CCCC1]

11. A pharmaceutical composition comprising a compound of claim 1 and a pharmaceutically acceptable carrier.

12-13. (canceled)

14. A compound of formula (II): 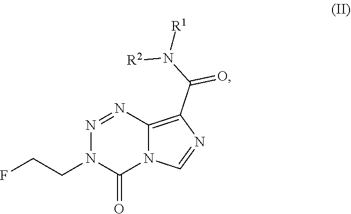
or optionally a pharmaceutically acceptable salt thereof,
wherein:
R¹ is selected from the group consisting of H and lower alkyl;
R² is selected from the group consisting of H, lower alkyl, trifluoroethyl, 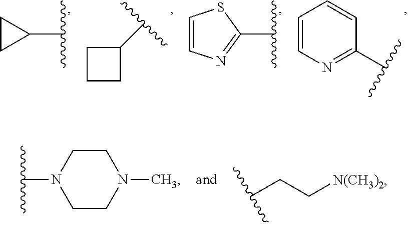
provided that R¹ is H when R² is other than H or lower alkyl; or R¹ and R² may combine to form —(CH₂)ₙ— or —(CH₂)₂—N(CH₃)—(CH₂)₂—;
n is 3, 4, or 5; and provided that R¹ and R² are not both H.

15. (canceled)

16. The compound of claim 14, wherein
R¹ is H, and
R² is trifluoroethyl,

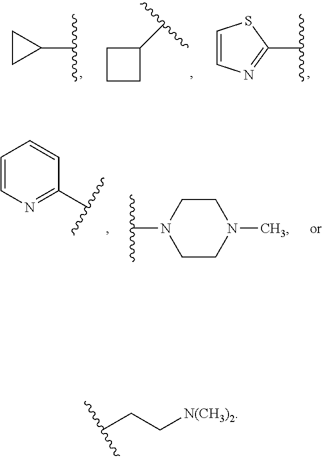

17. The compound of claim 14, wherein the compound is selected from the group consisting of: 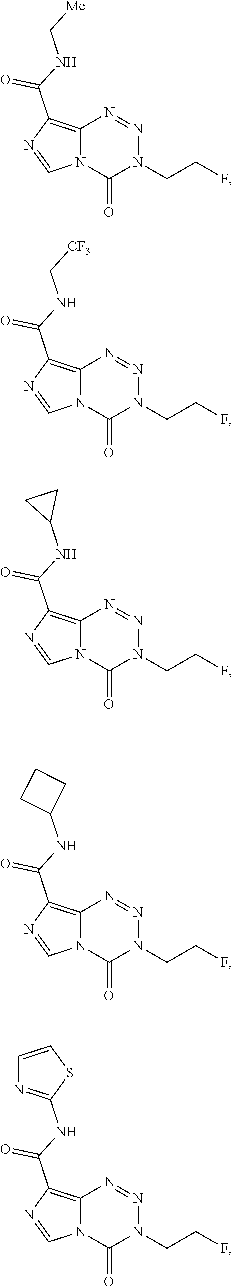 [SMILES: CCNC(=O)c1ncn2c(=O)n(CCF)nnc12 ; O=C(NCC(F)(F)F)c1ncn2c(=O)n(CCF)nnc12 ; O=C(NC1CC1)c1ncn2c(=O)n(CCF)nnc12 ; O=C(NC1CCC1)c1ncn2c(=O)n(CCF)nnc12 ; O=C(Nc1nccs1)c1ncn2c(=O)n(CCF)nnc12] 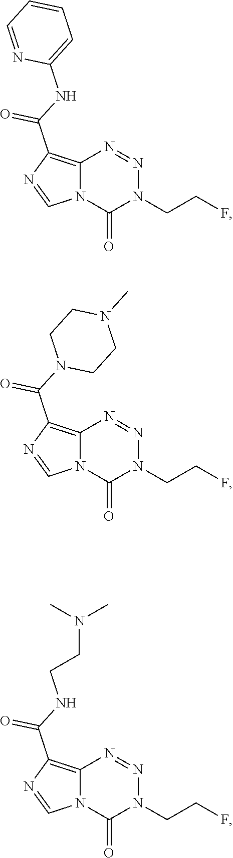 [SMILES: O=C(Nc1ccccn1)c1ncn2c(=O)n(CCF)nnc12 ; CN1CCN(C(=O)c2ncn3c(=O)n(CCF)nnc23)CC1 ; CN(C)CCNC(=O)c1ncn2c(=O)n(CCF)nnc12]
or a pharmaceutically acceptable salt thereof.

18. A compound selected from the group consisting of 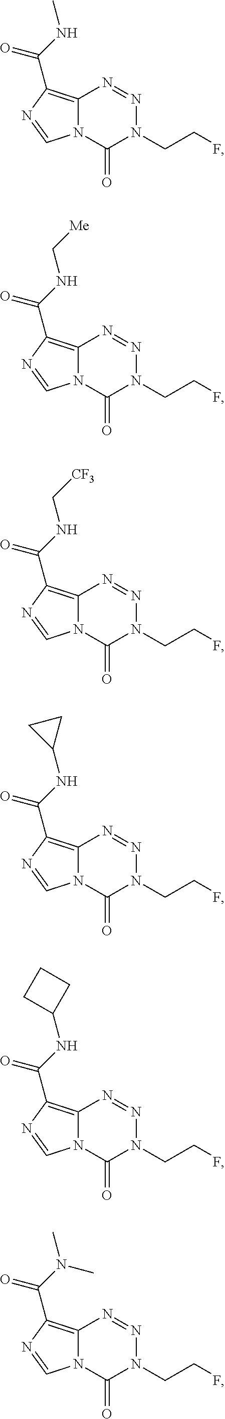 [SMILES: CNC(=O)c1ncn2c(=O)n(CCF)nnc12 ; CCNC(=O)c1ncn2c(=O)n(CCF)nnc12 ; O=C(NCC(F)(F)F)c1ncn2c(=O)n(CCF)nnc12 ; O=C(NC1CC1)c1ncn2c(=O)n(CCF)nnc12 ; O=C(NC1CCC1)c1ncn2c(=O)n(CCF)nnc12 ; CN(C)C(=O)c1ncn2c(=O)n(CCF)nnc12] 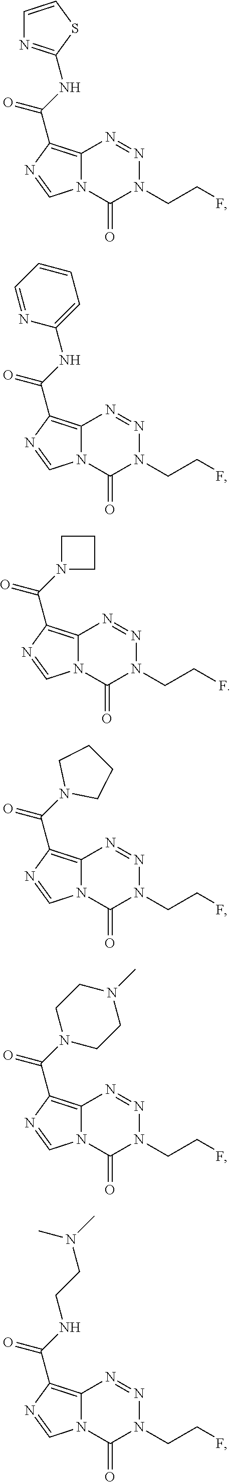 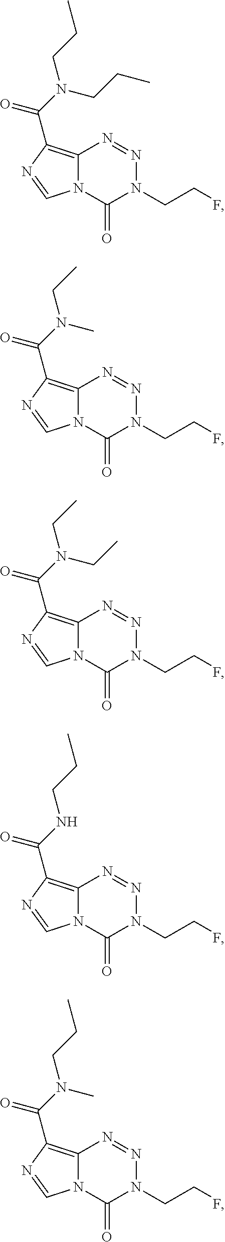 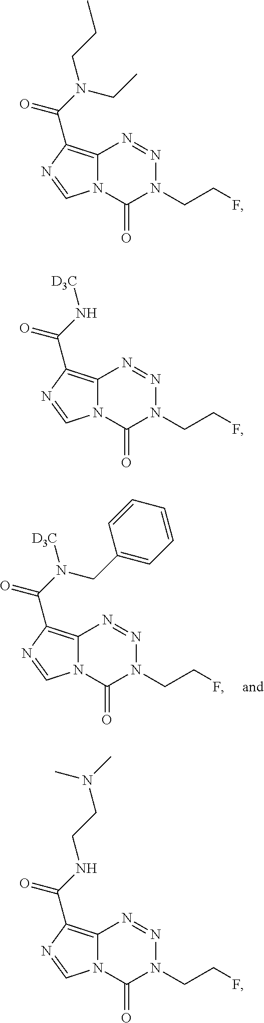
or a pharmaceutically acceptable salt thereof.

19. A pharmaceutical composition comprising a compound of claim 14 and a pharmaceutically acceptable carrier.

20. A method of treating, preventing, or ameliorating cancer in a subject in need thereof, the method comprising administering to the subject a therapeutically effective amount of a compound of claim 1, or a pharmaceutically acceptable salt thereof, optionally wherein the cancer is MGMT deficient and either MMR deficient or refractory to treatment with temozolomide.

21. (canceled)

22. The method of claim 20, wherein at least one of the following applies:
the cancer is a solid tumor, leukemia, or lymphoma;
the cancer is a brain tumor; and
the cancer is urothelial cancer, breast invasive carcinoma, colon adenocarcinoma, head and neck tumor (SCC), lung adenocarcinoma, rectum adenocarcinoma, acute myeloid leukemia, glioblastoma.

23-26. (canceled)

27. The method of claim 20, wherein the cancer is:
(i) selected from the group consisting of urothelial cancer, breast invasive carcinoma, colon adenocarcinoma, head and neck tumor (SCC), lung adenocarcinoma, rectum adenocarcinoma, acute myeloid leukemia, glioblastoma multiforme, and brain lower grade glioma, or
(ii) selected from the group consisting of glioma, colorectal cancer, non-small cell lung cancer, small cell lung cancer, sarcoma, pancreatic cancer, neuroendocrine tumor, esophageal cancer, lymphoma, head & neck cancer, breast cancer, bladder cancer, and leukemia,
optionally wherein the cancer is glioblastoma multiforme.

28-29. (canceled)

30. A method of treating, preventing, or ameliorating cancer in a subject in need thereof, wherein the method comprises administering to the subject an agent that induces DNA lesions in the cell that lead to irreparable DNA damage to selectively treat the cancer, wherein the cancer is characterized by a cancer cell having altered MGMT activity or having altered MGMT expression, optionally wherein the irreparable DNA damage is an unrepaired lesion, and optionally wherein the unrepaired lesion is a DNA inter- or intra-strand crosslink.

31-33. (canceled)

34. The method of claim 30, wherein at least one of the following applies:
i) the agent does not affect MGMT proficient tissue;
ii) the agent activity is independent of MMR protein expression and/or functional activity of the MMR pathway;
iii) the cancer is selected from the group consisting of a glioma, colorectal cancer, non-small cell lung cancer, small cell lung cancer, sarcoma, pancreatic cancer, neuroendocrine tumor, esophageal cancer, lymphoma, head & neck cancer, breast cancer, bladder cancer, and leukemia;
iv) the cancer is selected from the group consisting of an anaplastic astrocytoma, anaplastic oligodendroglioma, anaplastic ependymoma, medulloblastoma, and glioblastoma.

35-37. (canceled)

38. The method of claim 30, wherein the DNA lesion is a DNA double-strand break, a single-strand break, a stalled replication fork, a bulky adduct, or a lesion that further chemically reacts to form irreparable DNA damage, optionally wherein the irreparable DNA damage is an unrepaired lesion, optionally wherein the unrepaired lesion is a DNA inter- or intra-strand crosslink.

39. (canceled)

40. The method of claim 30, wherein the subject is resistant to treatment with an antineoplastic agent, optionally wherein the antineoplastic agent is selected from temozolomide, procarbazine, altretamine, dacarbazine, mitozolomide, cisplatin, carboplatin, dicycloplatin, eptaplatin, lobaplatin, oxaliplatin, miriplatin, nedaplatin, triplatin tetranitrate, phenanthriplatin, picoplatin, satraplatin, and lomustine.

41. (canceled)

42. The method of claim 30, wherein the agent is a compound of claim 14, optionally wherein the agent is an imidazotetrazine-based compound or a triazine-based compound.

43. (canceled)
